(19) United States
(12) Patent Application Publication
(10) Pub. No.: US 2024/0190963 A1
(43) Pub. Date: Jun. 13, 2024

(54) METHODS OF TREATING TUMOR

(71) Applicant: Bristol-Myers Squibb Company, Princeton, NJ (US)

(72) Inventors: Prabhu Seshaiyer Bhagavatheeswaran, Hamden, CT (US); Nicholas Allan John Botwood, Princeton, NJ (US); Han Chang, West Windsor, NJ (US); Yali Fu, Princeton, NJ (US); William J. Geese, Pipersville, PA (US); George A. Green, IV, Newton, NJ (US); Diane Healey, Madison, CT (US); Sabine Maier, Lawrenceville, NJ (US); Faith E. Nathan, Moorestown, NJ (US); Abderrahim Oukessou, Skillman, NJ (US); Giovanni Selvaggi, Brooklyn, NY (US); Joseph Daniel Szustakowski, Pennington, NJ (US)

(73) Assignee: Bristol-Myers Squibb Company, Princeton, NJ (US)

(21) Appl. No.: 18/468,097

(22) Filed: Sep. 15, 2023

Related U.S. Application Data
(63) Continuation of application No. 17/044,163, filed on Sep. 30, 2020, now abandoned.
(60) Provisional application No. 62/671,906, filed on May 15, 2018.
(60) Provisional application No. 62/650,845, filed on Mar. 30, 2018.

(51) Int. Cl. C07K 16/28; A61K 39/00; A61P 35/00; C07K 16/30

(52) U.S. Cl. CPC C07K 16/2818; A61P 35/00; C07K 16/2827; C07K 16/30; A61K 2039/507; A61K 2039/545; C07K 2317/76

(57) ABSTRACT

The disclosure provides a method for treating a subject afflicted with a tumor derived from a non-small cell lung cancer (NSCLC) comprising administering to the subject a therapeutically effective amount of (a) an anti-PD-1 antibody or antigen-binding portion thereof or an anti-PD-L1 antibody or antigen-binding portion thereof and (b) an anti-CTLA-4 antibody or an antigen binding portion thereof, wherein the tumor has a high tumor mutation burden (TMB) status. The TMB status can be determined by sequencing nucleic acids in the tumor and identifying a genomic alteration, e.g., a somatic nonsynonymous mutation, in the sequenced nucleic acids.

### FIELD OF THE DISCLOSURE

The present disclosure provides a method for treating a subject afflicted with a tumor derived from a non-small cell lung cancer (NSCLC) using an immunotherapy.

### SEQUENCE LISTING

The content of the electronically submitted sequence listing (Name: 3338_12100003_SequenceListing_ST26.xml; Size 13,492 bytes; and Date of Creation: Dec. 13, 2023) is herein incorporated by reference in its entirety.

### BACKGROUND OF THE DISCLOSURE

Human cancers harbor numerous genetic and epigenetic alterations, generating neoantigens potentially recognizable by the immune system (Sjoblom et al., Science (2006) 314(5797):268-274). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

Until recently, cancer immunotherapy had focused substantial effort on approaches that enhance anti-tumor immune responses by adoptive-transfer of activated effector cells, immunization against relevant antigens, or providing non-specific immune-stimulatory agents such as cytokines. In the past decade, however, intensive efforts to develop specific immune checkpoint pathway inhibitors have begun to provide new immunotherapeutic approaches for treating cancer, including the development of antibodies such as nivolumab and pembrolizumab (formerly lambrolizumab; USAN Council Statement, 2013) that bind specifically to the Programmed Death-1 (PD-1) receptor and block the inhibitory PD-1/PD-1 ligand pathway (Topalian et al., 2012a, b; Topalian et al., 2014; Hamid et al., 2013; Hamid and Carvajal, 2013; McDermott and Atkins, 2013).

PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to downregulate T cell activation and cytokine secretion upon binding to PD-1. Inhibition of the PD-1/PD-L1 interaction mediates potent antitumor activity in preclinical models (U.S. Pat. Nos. 8,008,449 and 7,943,743), and the use of antibody inhibitors of the PD-1/PD-L1 interaction for treating cancer has entered clinical trials (Brahmer et al., 2010; Topalian et al., 2012a; Topalian et al., 2014; Hamid et al., 2013; Brahmer et al., 2012; Flies et al., 2011; Pardoll, 2012; Hamid and Carvajal, 2013).

Nivolumab (formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014). Nivolumab has shown activity in a variety of advanced solid tumors, including renal cell carcinoma (renal adenocarcinoma, or hypernephroma), melanoma, and non-small cell lung cancer (NSCLC) (Topalian et al., 2012a; Topalian et al., 2014; Drake et al., 2013; WO 2013/173223).

The immune system and response to immuno-therapy are complex. Additionally, anti-cancer agents can vary in their effectiveness based on the unique patient characteristics. Accordingly, there is a need for targeted therapeutic strategies that identify patients who are more likely to respond to a particular anti-cancer agent and, thus, improve the clinical outcome for patients diagnosed with cancer.

### SUMMARY OF THE DISCLOSURE

Certain aspects of the present disclosure are directed to a method for treating a subject afflicted with a tumor derived from a non-small cell lung cancer (NSCLC) comprising administering to the subject a therapeutically effective amount of (a) an antibody or antigen-binding portion thereof that binds specifically to a Programmed Death-1 (PD-1) receptor and inhibits PD-1 activity (“an anti-PD-1 antibody”) or an antibody or antigen-binding portion thereof that binds specifically to a Programmed Death-Ligand 1 (PD-L1) and inhibits PD-1 activity (“an anti-PD-L1 antibody”) and (b) an antibody or antigen-binding portion thereof that binds specifically to cytotoxic T-lymphocyte-associated protein 4 (CTLA-4) (“an anti-CTLA-4 antibody”), wherein the tumor has a tumor mutation burden (TMB) status of at least about 10 mutations per megabase of genes examined. In some embodiments, the method further comprises measuring the TMB status of a biological sample obtained from the subject prior to the administering.

Some aspects of the present disclosure are directed to a method of identifying a subject who is afflicted with a tumor derived from a non-small cell lung cancer (NSCLC) and is suitable for a combination therapy of (a) an anti-PD-1 antibody or an anti-PD-L1 antibody and (b) an anti-CLTA-4 antibody, comprising measuring a TMB status of a biological sample of the subject, wherein the TMB status comprises at least about 10 mutations per megabase of genome examined and wherein the subject is identified as being suitable for the combination therapy. In some embodiments, the method further comprises administering to the subject a therapeutically effective amount of the anti-PD-1 antibody and the anti-CTLA-4 antibody.

In some embodiments, the TMB status is determined by sequencing nucleic acids in the tumor and identifying a genomic alteration in the sequenced nucleic acids. In some embodiments, the genomic alteration comprises one or more somatic mutations. In some embodiments, the genomic alteration comprises one or more nonsynonymous mutations. In some embodiments, the genomic alteration comprises one or more missense mutations. In some embodiments, the genomic alteration comprises one or more alterations selected from the group consisting of a base pair substitution, a base pair insertion, a base pair deletion, a copy number alteration (CNAs), a gene rearrangement, and any combination thereof.

In some embodiments, the TMB status of the tumor comprises at least 10 mutations, at least about 11 mutations, at least about 12 mutations, at least about 13 mutations, at least about 14 mutations, at least about 15 mutations, at least about 16 mutations, at least about 17 mutations, at least about 18 mutations, at least about 19 mutations, at least about 20 mutations, at least about 21 mutations, at least about 22 mutations, at least about 23 mutations, at least about 24 mutations, at least about 25 mutations, at least about 26 mutations, at least about 27 mutations, at least about 28 mutations, at least about 29 mutations, or at least about 30 mutations per megabase of genome examined as measured by a FOUNDATIONONE® CDX™ assay.

In some embodiments, the biological sample is a tumor tissue biopsy. In some embodiments, the tumor tissue is a formalin-fixed, paraffin-embedded tumor tissue or a fresh-frozen tumor tissue. In some embodiments, the biological sample is a liquid biopsy. In some embodiments, the biological sample comprises one or more of blood, serum, plasma, exoRNA, circulating tumor cells, ctDNA, and cfDNA.

In some embodiments, the TMB status is determined by genome sequencing. In some embodiments, the TMB status is determined by exome sequencing.

In some embodiments, the TMB status is determined by genomic profiling. In some embodiments, the genomic profile comprises at least about 20 genes, at least about 30 genes, at least about 40 genes, at least about 50 genes, at least about 60 genes, at least about 70 genes, at least about 80 genes, at least about 90 genes, at least about 100 genes, at least about 110 genes, at least about 120 genes, at least about 130 genes, at least about 140 genes, at least about 150 genes, at least about 160 genes, at least about 170 genes, at least about 180 genes, at least about 190 genes, at least about 200 genes, at least about 210 genes, at least about 220 genes, at least about 230 genes, at least about 240 genes, at least about 250 genes, at least about 260 genes, at least about 270 genes, at least about 280 genes, at least about 290 genes, at least about 300 genes, at least about 305 genes, at least about 310 genes, at least about 315 genes, at least about 320 genes, at least about 325 genes, at least about 330 genes, at least about 335 genes, at least about 340 genes, at least about 345 genes, at least about 350 genes, at least about 355 genes, at least about 360 genes, at least about 365 genes, at least about 370 genes, at least about 375 genes, at least about 380 genes, at least about 385 genes, at least about 390 genes, at least about 395 genes, or at least about 400 genes. In some embodiments, the genomic profile comprises at least about 265 genes. In some embodiments, the genomic profile comprises at least about 315 genes. In some embodiments, the genomic profile comprises at least about 354 genes.

In some embodiments, the genomic profile comprises one or more genes selected from the group consisting of ABL1, BRAF, CHEK1, FANCC, GATA3, JAK2, MITF, PDCD1LG2 (PD-L2), RBM10, STAT4, ABL2, BRCA1, CHEK2, FANCD2, GATA4, JAK3, MLH1, PDGFRA, RET, STK11, ACVR1B, BRCA2, CIC, FANCE, GATA6, JUN, MPL, PDGFRB, RICTOR, SUFU, AKT1, BRD4, CREBBP, FANCF, GID4 (C17orf 39), KAT6A (MYST 3), MRE 11A, PDK1, RNF43, SYK, AKT2, BRIP1, CRKL, FANCG, GLl1, KDM5A, MSH2, PIK3C2B, ROS1, TAF1, AKT3, BTG1, CRLF2, FANCL, GNA11, KDM5C, MSH6, PIK3CA, RPTOR, TBX3, ALK, BTK, CSF1R, FAS, GNA13, KDM6A, MTOR, PIK3CB, RUNX1, TERC, AMER1 (FAM123B), C11orf 30 (EMSY), CTCF, FAT1, GNAQ, KDR, MUTYH, PIK3CG, RUNX1T1, TERT (Promoter only), APC, CARD11, CTNNA1, FBXW7, GNAS, KEAP1, MYC, PIK3R1, SDHA, TET2, AR, CBFB, CTNN B1, FGF10, GPR124, KEL, MYCL (MYC L1), PIK3R2, SDHB, TGFBR2, ARAF, CBL, CUL3, FGF14, GRIN2A, KIT, MYCN, PLCG2, SDHC, TNFAIP3, ARFRP1, CCND1, CYLD, FGF19, GRM3, KLHL6, MYD88, PMS2, SDHD, TNFRSF14, ARID1A, CCND2, DAXX FGF23, GSK3B, KMT2A (MLL), NF1, POLD1, SETD2, TOP1, ARID1B, CCND3, DDR2, FGF3, H3F3A, KMT2C (MLL3), NF2, POLE, SF3B1, TOP2A, ARID2, CCNE1, DICER1, FGF4, HGF, KMT2D (MLL2), NFE2L2, PPP2R1A, SLIT2, TP53, ASXL1, CD274 (PD-L1), DNMT3A, FGF6, HNF1A, KRAS, NFKB1A, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX, CDC73, EP300, FGFR3, HSP90AA1, LYN, NOTCH2, PRKC1, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLCN, IGF1R, MAP2K1 (MEK1), NRAS, PTCH1, SNCAIP, WISP3, AXL, CDK6, EPHB1, FLT1, IGF2, MAP2K2 (MEK2), NSD1, PTEN, SOCS1, WT1, BAP1, CDK8, ERBB2, FLT3, IKBKE, MAP2K4, NTRK1, PTPN11, SOX10, XPO1, BARD1, CDKN1A, ERBB3, FLT4, IKZF1, MAP3K1, NTRK2, QKI, SOX2, ZBTB2, BCL2, CDKN1B, ERBB4, FOXL2, IL7R, MCL1, NTRK3, RAC1, SOX9, ZNF217, BCL2L1, CDKN2A, ERG, FOXP1, INHBA, MDM2, NUP93, RAD50, SPEN, ZNF703, BCL2L2, CDKN2B, ERRFl1, FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRM1, RB1, STAT3, and any combination thereof.

In some embodiments, the TMB status is measured by a FOUNDATIONONE® CDX™ assay.

In some embodiments, the method further comprises identifying a genomic alteration in one or more of ETV4, TMPRSS2, ETV5, BCR, ETV1, ETV6, and MYB.

In some embodiments, the tumor has a high neoantigen load. In some embodiments, the subject has an increased T-cell repertoire.

Certain aspects of the present disclosure are directed to a method for treating a subject afflicted with a tumor derived from a non-small cell lung cancer (NSCLC) comprising: (i) measuring a TMB status of the tumor by a FOUNDATIONONE® CDX™ assay, (ii) administering to the subject a therapeutically effective amount of an anti-PD-1 antibody and an anti-CTLA-4 antibody, wherein the TMB status has at least about 10 mutations per megabase of genome examined.

In some embodiments, the NSCLC has a squamous histology. In some embodiments, the NSCLC has a non-squamous histology.

In some embodiments, the anti-PD-1 antibody cross-competes with nivolumab or pembrolizumab for binding to human PD-1. In some embodiments, the anti-PD-1 antibody binds to the same epitope as nivolumab or pembrolizumab. In some embodiments, the anti-PD-1 antibody is a chimeric antibody, a humanized antibody, or a human monoclonal antibody. In some embodiments, the anti-PD-1 antibody comprises a heavy chain constant region of a human IgG1 isotype or a human IgG4 isotype. In some embodiments, the anti-PD-1 antibody is nivolumab. In some embodiments, the anti-PD-1 antibody is pembrolizumab.

In some embodiments, the anti-PD-1 antibody is administered at a dose ranging from 0.1 mg/kg to 20.0 mg/kg body weight once every 2, 3, or 4 weeks. In some embodiments, the anti-PD-1 antibody is administered at a dose of 2 mg/kg body weight once every 3 weeks. In some embodiments, the anti-PD-1 antibody is administered at a dose of 3 mg/kg body weight once every 2 weeks.

In some embodiments, the therapeutically effective amount of the anti-PD-1 antibody is a flat dose. In some embodiments, the therapeutically effective amount of the anti-PD-1 antibody is a flat dose of at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, or at least about 550 mg. In some embodiments, the anti-PD-1 antibody is administered as a flat dose about once every 1, 2, 3, or 4 weeks. In some embodiments, the anti-PD-1 antibody is administered as a flat dose of about 200 mg once every 3 weeks. In some embodiments, the anti-PD-1 antibody is administered as a flat dose of about 240 mg once every 2 weeks. In some embodiments, the anti-PD-1 antibody is administered as a flat dose of about 480 mg once every 4 weeks.

In some embodiments, the anti-PD-L1 antibody cross-competes with durvalumab, avelumab, or atezolizumab for binding to human PD-1. In some embodiments, the anti-PD-L1 antibody binds to the same epitope as durvalumab, avelumab, or atezolizumab. In some embodiments, the anti-PD-L1 antibody is durvalumab. In some embodiments, the anti-PD-L1 antibody is avelumab. In some embodiments, the anti-PD-L1 antibody is atezolizumab.

In some embodiments, the anti-PD-L1 antibody is administered at a dose ranging from 0.1 mg/kg to 20.0 mg/kg body weight once every 2, 3, or 4 weeks. In some embodiments, the anti-PD-L1 antibody is administered at a dose of 15 mg/kg body weight once every 3 weeks. In some embodiments, the anti-PD-L1 antibody is administered at a dose of 10 mg/kg body weight once every 2 weeks.

In some embodiments, the therapeutically effective amount of the anti-PD-L1 antibody is a flat dose. In some embodiments, the therapeutically effective amount of the anti-PD-L1 antibody is a flat dose of at least about 240 mg, at least about 300 mg, at least about 320 mg, at least about 400 mg, at least about 480 mg, at least about 500 mg, at least about 560 mg, at least about 600 mg, at least about 640 mg, at least about 700 mg, at least 720 mg, at least about 800 mg, at least about 880 mg, at least about 900 mg, at least 960 mg, at least about 1000 mg, at least about 1040 mg, at least about 1100 mg, at least about 1120 mg, at least about 1200 mg, at least about 1280 mg, at least about 1300 mg, at least about 1360 mg, or at least about 1400 mg. In some embodiments, the anti-PD-L1 antibody is administered as a flat dose about once every 1, 2, 3, or 4 weeks. In some embodiments, the anti-PD-L1 antibody is administered as a flat dose of about 1200 mg once every 3 weeks. In some embodiments, the anti-PD-L1 antibody is administered as a flat dose of about 800 mg once every 2 weeks.

In some embodiments, the anti-CTLA-4 antibody cross-competes with for binding to human CTLA-4. In some embodiments, the anti-CTLA-4 antibody binds to the same epitope as ipilimumab or tremelimumab. In some embodiments, the anti-CTLA-4 antibody is ipilimumab. In some embodiments, the anti-CTLA-4 antibody is tremelimumab.

In some embodiments, the anti-CTLA-4 antibody is administered at a dose ranging from 0.1 mg/kg to 20.0 mg/kg body weight once every 2, 3, 4, 5, 6, 7, or 8 weeks. In some embodiments, the anti-CTLA-4 antibody is administered at a dose of 1 mg/kg body weight once every 6 weeks. In some embodiments, the anti-CTLA-4 antibody is administered at a dose of 1 mg/kg body weight once every 4 weeks.

In some embodiments, the therapeutically effective amount of the anti-CTLA-4 antibody is a flat dose. In some embodiments, the therapeutically effective amount of the anti-CTLA-4 antibody is a flat dose of at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, or at least about 200 mg. In some embodiments, the anti-CLTA-4 antibody is administered as a flat dose about once every 2, 3, 4, 5, 6, 7, or 8 weeks.

In some embodiments, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the administration.

In some embodiments, the subject exhibits an overall survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the administration.

In some embodiments, the subject exhibits an objective response rate of at least about 30%, about 35%, about 40%, about 45%, about 50%, about 55%, about 60%, about 65%, about 70%, about 75%, about 80%, about 85%, about 90%, about 95%, or about 100%.

In some embodiments, less than 1% of the tumor cells express PD-L1.

Other features and advantages of the instant disclosure will be apparent from the following detailed description and examples which should not be construed as limiting. The contents of all cited references, including scientific articles, newspaper reports, GenBank entries, patents and patent applications cited throughout this application are expressly incorporated herein by reference.

### Embodiments

E1. A method for treating a subject afflicted with a tumor derived from a non-small cell lung cancer (NSCLC) comprising administering to the subject a therapeutically effective amount of (a) an antibody or antigen-binding portion thereof that binds specifically to a Programmed Death-1 (PD-1) receptor and inhibits PD-1 activity (“an anti-PD-1 antibody”) or an antibody or antigen-binding portion thereof that binds specifically to a Programmed Death-Ligand 1 (PD-L1) and inhibits PD-1 activity (“an anti-PD-L1 antibody”) and (b) an antibody or antigen-binding portion thereof that binds specifically to cytotoxic T-lymphocyte-associated protein 4 (CTLA-4) (“an anti-CTLA-4 antibody”), wherein the tumor has a tumor mutation burden (TMB) status of at least about 10 mutations per megabase of genes examined.

E2. The method of E1, further comprising measuring the TMB status of a biological sample obtained from the subject prior to the administering.

E3. A method of identifying a subject who is afflicted with a tumor derived from a non-small cell lung cancer (NSCLC) and is suitable for a combination therapy of (a) an anti-PD-1 antibody or an anti-PD-L1 antibody and (b) an anti-CLTA-4 antibody, comprising measuring a TMB status of a biological sample of the subject, wherein the TMB status comprises at least about 10 mutations per megabase of genome examined and wherein the subject is identified as being suitable for the combination therapy.

E4. The method of E3, further comprising administering to the subject a therapeutically effective amount of the anti-PD-1 antibody and the anti-CTLA-4 antibody.

E5. The method of any one of E1 to E4, wherein the TMB status is determined by sequencing nucleic acids in the tumor and identifying a genomic alteration in the sequenced nucleic acids.

E6. The method of E5, wherein the genomic alteration comprises one or more somatic mutations.

E7. The method of E5 or E6, wherein the genomic alteration comprises one or more nonsynonymous mutations.

E8. The method of any one of E5 to E7, wherein the genomic alteration comprises one or more missense mutations.

E9. The method of any one of E5 to E8, wherein the genomic alteration comprises one or more alterations selected from the group consisting of a base pair substitution, a base pair insertion, a base pair deletion, a copy number alteration (CNAs), a gene rearrangement, and any combination thereof.

E10. The method of any one of E1 to E9, wherein the TMB status of the tumor comprises at least 10 mutations, at least about 11 mutations, at least about 12 mutations, at least about 13 mutations, at least about 14 mutations, at least about 15 mutations, at least about 16 mutations, at least about 17 mutations, at least about 18 mutations, at least about 19 mutations, at least about 20 mutations, at least about 21 mutations, at least about 22 mutations, at least about 23 mutations, at least about 24 mutations, at least about 25 mutations, at least about 26 mutations, at least about 27 mutations, at least about 28 mutations, at least about 29 mutations, or at least about 30 mutations per megabase of genome examined as measured by a FOUNDATIONONE® CDX™ assay.

E11. The method of any one of E2 to E10, wherein the biological sample is a tumor tissue biopsy.

E12. The method of E11, wherein the tumor tissue is a formalin-fixed, paraffin-embedded tumor tissue or a fresh-frozen tumor tissue.

E13. The method of any one of E2 to E11, wherein the biological sample is a liquid biopsy.

E14. The method of any one of E2 to E11, wherein the biological sample comprises one or more of blood, serum, plasma, exoRNA, circulating tumor cells, ctDNA, and cfDNA.

E15. The method of any one of E1 to E14, wherein the TMB status is determined by genome sequencing.

E16. The method of any one of E1 to E14, wherein the TMB status is determined by exome sequencing.

E17. The method of any one of E1 to E14, wherein the TMB status is determined by genomic profiling.

E18. The method of E17, wherein the genomic profile comprises at least about 20 genes, at least about 30 genes, at least about 40 genes, at least about 50 genes, at least about 60 genes, at least about 70 genes, at least about 80 genes, at least about 90 genes, at least about 100 genes, at least about 110 genes, at least about 120 genes, at least about 130 genes, at least about 140 genes, at least about 150 genes, at least about 160 genes, at least about 170 genes, at least about 180 genes, at least about 190 genes, at least about 200 genes, at least about 210 genes, at least about 220 genes, at least about 230 genes, at least about 240 genes, at least about 250 genes, at least about 260 genes, at least about 270 genes, at least about 280 genes, at least about 290 genes, at least about 300 genes, at least about 305 genes, at least about 310 genes, at least about 315 genes, at least about 320 genes, at least about 325 genes, at least about 330 genes, at least about 335 genes, at least about 340 genes, at least about 345 genes, at least about 350 genes, at least about 355 genes, at least about 360 genes, at least about 365 genes, at least about 370 genes, at least about 375 genes, at least about 380 genes, at least about 385 genes, at least about 390 genes, at least about 395 genes, or at least about 400 genes.

E19. The method of E17, wherein the genomic profile comprises at least about 265 genes.

E20. The method of E17, wherein the genomic profile comprises at least about 315 genes.

E21. The method of E17, wherein the genomic profile comprises at least about 354 genes.

E22. The method of E17 or 18, wherein the genomic profile comprises one or more genes selected from the group consisting of ABL1, BRAF, CHEK1, FANCC, GATA3, JAK2, MITF, PDCD1LG2 (PD-L2), RBM10, STAT4, ABL2, BRCA1, CHEK2, FANCD2, GATA4, JAK3, MLH1, PDGFRA, RET, STK11, ACVR1B, BRCA2, CIC, FANCE, GATA6, JUN, MPL, PDGFRB, RICTOR, SUFU, AKT1, BRD4, CREBBP, FANCF, GID4 (C17orf 39), KAT6A (MYST 3), MRE 11A, PDK1, RNF43, SYK, AKT2, BRIP1, CRKL, FANCG, GLl1, KDM5A, MSH2, PIK3C2B, ROS1, TAF1, AKT3, BTG1, CRLF2, FANCL, GNA11, KDM5C, MSH6, PIK3CA, RPTOR, TBX3, ALK, BTK, CSF1R, FAS, GNA13, KDM6A, MTOR, PIK3CB, RUNX1, TERC, AMER1 (FAM123B), C11orf 30 (EMSY), CTCF, FAT1, GNAQ, KDR, MUTYH, PIK3CG, RUNX1T1, TERT (Promoter only), APC, CARD11, CTNNA1, FBXW7, GNAS, KEAP1, MYC, PIK3R1, SDHA, TET2, AR, CBFB, CTNN B1, FGF10, GPR124, KEL, MYCL (MYC L1), PIK3R2, SDHB, TGFBR2, ARAF, CBL, CUL3, FGF14, GRIN2A, KIT, MYCN, PLCG2, SDHC, TNFAIP3, ARFRP1, CCND1, CYLD, FGF19, GRM3, KLHL6, MYD88, PMS2, SDHD, TNFRSF14, ARID1A, CCND2, DAXX, FGF23, GSK3B, KMT2A (MLL), NF1, POLD1, SETD2, TOP1, ARID1B, CCND3, DDR2, FGF3, H3F3A, KMT2C (MLL3), NF2, POLE, SF3B1, TOP2A, ARID2, CCNE1, DICER1, FGF4, HGF, KMT2D (MLL2), NFE2L2, PPP2R1A, SLIT2, TP53, ASXL1, CD274 (PD-L1), DNMT3A, FGF6, HNF1A, KRAS, NFKB1A, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX, CDC73, EP300, FGFR3, HSP90AA1, LYN, NOTCH2, PRKC1, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLCN, IGF1R, MAP2K1 (MEK1), NRAS, PTCH1, SNCAIP, WISP3, AXL, CDK6, EPHB1, FLT1, IGF2, MAP2K2 (MEK2), NSD1, PTEN, SOCS1, WT1, BAP1, CDK8, ERBB2, FLT3, IKBKE, MAP2K4, NTRK1, PTPN11, SOX10, XPO1, BARD1, CDKN1A, ERBB3, FLT4, IKZF1, MAP3K1, NTRK2, QKI, SOX2, ZBTB2, BCL2, CDKN1B, ERBB4, FOXL2, IL7R, MCL1, NTRK3, RAC1, SOX9, ZNF217, BCL2L1, CDKN2A, ERG, FOXP1, INHBA, MDM2, NUP93, RAD50, SPEN, ZNF703, BCL2L2, CDKN2B, ERRFl1, FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRM1, RB1, STAT3, and any combination thereof.

E23. The method of any one of E1 to E22, wherein the TMB status is measured by a FOUNDATIONONE® CDX™ assay.

E24. The method of any one of E1 to E23, further comprising identifying a genomic alteration in one or more of ETV4, TMPRSS2, ETV5, BCR, ETV1, ETV6, and MYB.

E25. The method of any one of E1 to E24, wherein the tumor has a high neoantigen load.

E26. The method of any one of E1 to E25, wherein the subject has an increased T-cell repertoire.

E27. A method for treating a subject afflicted with a tumor derived from a non-small cell lung cancer (NSCLC) comprising: (i) measuring a TMB status of the tumor by a FOUNDATIONONE® CDX™ assay, (ii) administering to the subject a therapeutically effective amount of an anti-PD-1 antibody and an anti-CTLA-4 antibody, wherein the TMB status has at least about 10 mutations per megabase of genome examined.

E28. The method of any one of E1 to E27, wherein the NSCLC has a squamous histology.

E29. The method of any one of E1 to E27, wherein the NSCLC has a non-squamous histology.

E30. The method of any one of E1 to E29, wherein the anti-PD-1 antibody cross-competes with nivolumab or pembrolizumab for binding to human PD-1.

E31. The method of any one of E1 to E29, wherein the anti-PD-1 antibody binds to the same epitope as nivolumab or pembrolizumab.

E32. The method of any one of E1 to E30, wherein the anti-PD-1 antibody is a chimeric antibody, a humanized antibody, or a human monoclonal antibody.

E33. The method of any one of E1 to E32, wherein the anti-PD-1 antibody comprises a heavy chain constant region of a human IgG1 isotype or a human IgG4 isotype.

E34. The method of any one of E1 to E33, wherein the anti-PD-1 antibody is nivolumab.

E35. The method of any one of E1 to E33, wherein the anti-PD-1 antibody is pembrolizumab.

E36. The method of any one of E1 to E35, wherein the anti-PD-1 antibody is administered at a dose ranging from 0.1 mg/kg to E20.0 mg/kg body weight once every 2, 3, or 4 weeks.

E37. The method of any one of E1 to E36, wherein the anti-PD-1 antibody is administered at a dose of 2 mg/kg body weight once every 3 weeks.

E38. The method of any one of E1 to E36, wherein the anti-PD-1 antibody is administered at a dose of 3 mg/kg body weight once every 2 weeks.

E39. The method of any one of E1 to E35, wherein the therapeutically effective amount of the anti-PD-1 antibody is a flat dose.

E40. The method of E39, wherein the therapeutically effective amount of the anti-PD-1 antibody is a flat dose of at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, or at least about 550 mg.

E41. The method of E39 or E40, wherein the anti-PD-1 antibody is administered as a flat dose about once every 1, 2, 3, or 4 weeks.

E42. The method of any one of E1 to E35, wherein the anti-PD-1 antibody is administered as a flat dose of about 200 mg once every 3 weeks.

E43. The method of any one of E1 to E35, wherein the anti-PD-1 antibody is administered as a flat dose of about 240 mg once every 2 weeks.

E44. The method of any one of E1 to E35, wherein the anti-PD-1 antibody is administered as a flat dose of about 480 mg once every 4 weeks.

E45. The method of any one of E1 to E29, wherein the anti-PD-L1 antibody cross-competes with durvalumab, avelumab, or atezolizumab for binding to human PD-1.

E46. The method of any one of E1 to E29, wherein the anti-PD-L1 antibody binds to the same epitope as durvalumab, avelumab, or atezolizumab.

E47. The method of any one of E1 to E29, wherein the anti-PD-L1 antibody is durvalumab.

E48. The method of any one of E1 to E29, wherein the anti-PD-L1 antibody is avelumab.

E49. The method of any one of E1 to E29, wherein the anti-PD-L1 antibody is atezolizumab.

E50. The method of any one of E45 to E49, wherein the anti-PD-L1 antibody is administered at a dose ranging from 0.1 mg/kg to E20.0 mg/kg body weight once every 2, 3, or 4 weeks.

E51. The method of any one of E45 to E49, wherein the anti-PD-L1 antibody is administered at a dose of 15 mg/kg body weight once every 3 weeks.

E52. The method of any one of E45 to E49, wherein the anti-PD-L1 antibody is administered at a dose of 10 mg/kg body weight once every 2 weeks.

E53. The method of any one of E1 to E29 and E45 to E49, wherein the therapeutically effective amount of the anti-PD-L1 antibody is a flat dose.

E54. The method of E53, wherein the therapeutically effective amount of the anti-PD-L1 antibody is a flat dose of at least about 240 mg, at least about 300 mg, at least about 320 mg, at least about 400 mg, at least about 480 mg, at least about 500 mg, at least about 560 mg, at least about 600 mg, at least about 640 mg, at least about 700 mg, at least 720 mg, at least about 800 mg, at least about 880 mg, at least about 900 mg, at least 960 mg, at least about 1000 mg, at least about 1040 mg, at least about 1100 mg, at least about 1120 mg, at least about 1200 mg, at least about 1280 mg, at least about 1300 mg, at least about 1360 mg, or at least about 1400 mg.

E55. The method of E53 or E54, wherein the anti-PD-L1 antibody is administered as a flat dose about once every 1, 2, 3, or 4 weeks.

E56. The method of any one of E53 to E55, wherein the anti-PD-L1 antibody is administered as a flat dose of about 1200 mg once every 3 weeks.

E57. The method of any one of E53 to E55, wherein the anti-PD-L1 antibody is administered as a flat dose of about 800 mg once every 2 weeks.

E58. The method of any one of E1 to E57, wherein the anti-CTLA-4 antibody cross-competes with for binding to human CTLA-4.

E59. The method of any one of E1 to E57, wherein the anti-CTLA-4 antibody binds to the same epitope as ipilimumab or tremelimumab.

E60. The method of any one of E1 to E59, wherein the anti-CTLA-4 antibody is ipilimumab.

E61. The method of any one of E1 to E59, wherein the anti-CTLA-4 antibody is tremelimumab.

E62. The method of any one of E1 to E59, wherein the anti-CTLA-4 antibody is administered at a dose ranging from 0.1 mg/kg to E20.0 mg/kg body weight once every 2, 3, 4, 5, 6, 7, or 8 weeks.

E63. The method of any one of E1 to E59, wherein the anti-CTLA-4 antibody is administered at a dose of 1 mg/kg body weight once every 6 weeks.

E64. The method of any one of E1 to E59, wherein the anti-CTLA-4 antibody is administered at a dose of 1 mg/kg body weight once every 4 weeks.

E65. The method of any one of E1 to E61, wherein the therapeutically effective amount of the anti-CTLA-4 antibody is a flat dose.

E66. The method of E65, wherein the therapeutically effective amount of the anti-CTLA-4 antibody is a flat dose of at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, or at least about 200 mg.

E67. The method of E65 or E66, wherein the anti-CLTA-4 antibody is administered as a flat dose about once every 2, 3, 4, 5, 6, 7, or 8 weeks.

E68. The method of any one of E1 to E67, wherein the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the administration.

E69. The method of any one of E1 to E68, wherein the subject exhibits an overall survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the administration.

E70. The method of any one of E1 to E69, wherein the subject exhibits an objective response rate of at least about 30%, about 35%, about 40%, about 45%, about 50%, about 55%, about 60%, about 65%, about 70%, about 75%, about 80%, about 85%, about 90%, about 95%, or about 100%.

E71. The method of any one of E1 to E70, wherein the tumor is PD-L1 negative.

E72. The method of any one of E1 to E71, wherein the tumor has less than 1% of PD-L1.

### BRIEF DESCRIPTION OF THE DRAWINGS

FIG. 1 shows the study design of treating NSCLC. The subjects were divided up by the PD-L1 expression status, i.e., ≥1% PD-L1 expression v. <PD-L1 expression. The subjects in each group were then divided up into three groups (1:1:1) receiving (i) an anti-PD-1 antibody (e.g., nivolumab) at a dose of 3 mg/kg q2Q and an anti-CTLA-4 antibody, e.g., ipilimumab, at a dose of mg/kg q6W (n=396 or n=187); (ii) histology-based chemotherapy (n=397 or n=186), and (iii) an anti-PD-1 antibody, e.g., nivolumab, alone at a flat dose of 240 mg q2W (n=396 or n=177). The subjects who were receiving histology-based chemotherapy were further stratified by its status, i.e., squamous (SQ) NSCLC or non-squamous (NSQ) NSCLC. The subjects with NSQ NSCLC who received a chemotherapy received pemetrexed (500 mg/m2)+cisplatin (75 mg/m2) or carboplatin (AUC 5 or 6), Q3W for ≤4 cycles, with optional pemetrexed (500 mg/m2) maintenance following chemotherapy or nivolumab (360 mg Q3W)+pemetrexed (500 mg/m2) maintenance following nivolumab+chemotherapy. The subjects with SQ NSCLC who received a chemotherapy received gemcitabine (1000 or 1250 mg/m2)+cisplatin (75 mg/m2), or gemcitabine (1000 mg/m2)+carboplatin (AUC 5), Q3W for ≤4 cycles. The TBM co-primary analysis was conducted in the subset of patients randomized to nivolumab+ipilimumab or chemotherapy who had evaluable TMB≥10 mutations/Mb.

FIG. 2 shows a scatterplot of TMB and PD-L1 Expression in all TMB-evaluable Patients. The y axis shows the number of mutations per megabase, and the x axis shows PD-L1 expression. Symbols (dots) in the scatterplot may represent multiple data points, especially for patients with <1% PD-L1 expression.

FIG. 3A shows progression-free survival with an anti-PD-1 antibody (e.g., nivolumab) plus an anti-CLTA-4 antibody (e.g., Ipilimumab) vs. chemotherapy in all randomized patients. Cl shows confidence interval; HR shows hazard ratio. FIG. 3B shows progression-free survival with an anti-PD-1 antibody (e.g., nivolumab) plus an anti-CLTA-4 antibody (e.g., Ipilimumab) vs. chemotherapy in TMB evaluable patients.

FIG. 4A shows progression-free survival of an anti-PD-1 antibody (e.g., nivolumab) plus an anti-CLTA-4 antibody (e.g., Ipilimumab) (Nivo+Ipi) vs. chemotherapy (Chemo) in patients with TMB≥10 mutations/Mb. 1-y PFS=progression-free survival at one year; *95% CI, 0.43 to 0.77.

FIG. 4B shows duration of response of an anti-PD-1 antibody (e.g., nivolumab) plus an anti-CLTA-4 antibody (e.g., Ipilimumab) (Nivo+Ipi) vs. chemotherapy (Chemo) in patients with TMB≥10 mutations/Mb. DOR: duration of response; Median, DOR, mo: median month of duration of response; 1-y DOR: duration of response at one year.

FIG. 5 shows Progression-free Survival with an anti-PD-1 antibody (e.g., nivolumab) plus an anti-CLTA-4 antibody (e.g., Ipilimumab) vs. chemotherapy in patients With TMB<10 mutations/Mb.

FIG. 6A shows subgroup analyses of progression-free survival in patients with TMB≥10 mutations/Mb by PD-L1 expression ≥1%. PFS (%): percentage of progression-free survival. FIG. 6B shows subgroup analyses of progression-free survival in patients with TMB≥10 mutations/Mb by PD-L1 expression <1%. FIG. 6C shows subgroup analyses of progression-free survival in patients with TMB≥10 mutations/Mb in patients with squamous cell tumor histology. FIG. 6D shows subgroup analyses of progression-free survival in patients with TMB≥10 mutations/Mb in patients with non-squamous cell tumor histology. FIG. 6E shows the characteristics of the selected subgroups.

FIG. 7 shows progression-free Survival with an anti-PD-1 antibody (e.g., nivolumab) monotherapy vs. chemotherapy in patients with TMB≥13 mutations/Mb and ≥1% tumor PD-L1 expression. 95% Cl is 0.95 (0.64, 1.4).

FIG. 8 shows progression-free survival with an anti-PD-1 antibody (e.g., nivolumab) plus an anti-CLTA-4 antibody (e.g., Ipilimumab) vs. an anti-PD-1 antibody (e.g., nivolumab) monotherapy and chemotherapy in patients with TMB≥10 mutations/Mb and ≥1% tumor PD-L1 expression. 95% CI is 0.62 (0.44, 0.88) for nivolumab+ipilimumab vs. chemotherapy.

FIGS. 9A-9C show the progression free survival (PFS; FIG. 9A), objective response rate (ORR; FIG. 9B), and duration of response (DOR; FIG. 9C) following treatment with either nivolumab+chemotherapy or chemotherapy alone for patients having <1% tumor PD-L1 expression.

FIG. 9D shows the stratification of the patients based on baseline characteristics and the associated unstratified hazard ratios (HR) following treatment with either nivolumab+chemotherapy (“Nivo+Chemo”) or chemotherapy alone (“Chemo”).

FIGS. 10A-10B show the progression free survival (PFS) for high TMB (≥10 mut/Mb; FIG. 10A) and low TMB (<10 mut/Mb; FIG. 10B) patients having <1% tumor PD-L1 expression following treatment with nivolumab+ipilimumab (vertical dashes), nivolumab+chemotherapy (circles), or chemotherapy alone (triangles) (FIGS. 10A-10B). FIG. 10C shows the duration of response (DOR) for high TMB (10 mut/Mb) patients having <1% tumor PD-L1 expression following treatment with nivolumab+ipilimumab (vertical dashes), nivolumab+chemotherapy (circles), or chemotherapy alone (triangles).

FIG. 11 shows the distribution of select treatment-related adverse events (TRAEs) in patients treated with either nivolumab+chemotherapy (left of y axis) or nivolumab+ipilimumab (right of y axis). Dark grey and black bars indicate grade 1-2 TRAEs, and light grey bars indicate grade 3-4 TRAEs. ^{a}Select AEs are those with potential immunologic etiology that require frequent monitoring/intervention.

### DETAILED DESCRIPTION OF THE DISCLOSURE

The present disclosure provides a method for treating a subject afflicted with a tumor derived from non-small cell lung cancer (“NSCLC”) comprising administering to the subject a combination therapy comprising (a) an anti-PD-1 antibody or an anti-PD-L1 antibody and (b) an anti-CTLA-4 antibody, wherein the tumor has a high tumor mutation burden (TMB) status. In certain embodiments, the tumor has a TMB of at least about 10 mutations per megabase of genes examined.

The present disclosure also provides a method for identifying a subject afflicted with a tumor derived from a NSCLC suitable for a combination therapy of (a) an anti-PD-1 antibody or an anti-PD-L1 antibody and (b) an anti-CTLA-4 antibody, comprising measuring a TMB status of a biological sample of the tumor, wherein the tumor has a high TMB status, and wherein the subject is identified as being suitable for the combination therapy. In some embodiments, the subject identified as being suitable for the combination therapy has a tumor having a TMB of at least about 10 mutations per megabase of genes examined.

### Terms

In order that the present disclosure can be more readily understood, certain terms are first defined. As used in this application, except as otherwise expressly provided herein, each of the following terms shall have the meaning set forth below. Additional definitions are set forth throughout the application.

“Administering” refers to the physical introduction of a composition comprising a therapeutic agent to a subject, using any of the various methods and delivery systems known to those skilled in the art. Preferred routes of administration for the immunotherapy, e.g., the anti-PD-1 antibody or the anti-PD-L1 antibody, include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include an oral, topical, epidermal or mucosal route of administration, for example, intranasally, vaginally, rectally, sublingually or topically. Administering can also be performed, for example, once, a plurality of times, and/or over one or more extended periods.

An “adverse event” (AE) as used herein is any unfavorable and generally unintended or undesirable sign (including an abnormal laboratory finding), symptom, or disease associated with the use of a medical treatment. For example, an adverse event can be associated with activation of the immune system or expansion of immune system cells (e.g., T cells) in response to a treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 may, however, have cross-reactivity to other antigens, such as PD-1 molecules from different species. Moreover, an isolated antibody can be substantially free of other cellular material and/or chemicals.

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic or other techniques known to those skilled in the art.

A “human antibody” (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibody” and “fully human antibody” and are used synonymously.

A “humanized antibody” refers to an antibody in which some, most or all of the amino acids outside the CDRs of a non-human antibody are replaced with corresponding amino acids derived from human immunoglobulins. In one embodiment of a humanized form of an antibody, some, most or all of the amino acids outside the CDRs have been replaced with amino acids from human immunoglobulins, whereas some, most or all amino acids within one or more CDRs are unchanged. Small additions, deletions, insertions, substitutions or modifications of amino acids are permissible as long as they do not abrogate the ability of the antibody to bind to a particular antigen. A “humanized antibody” retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen antibody” refers to an antibody that binds specifically to the antigen. For example, an anti-PD-1 antibody binds specifically to PD-1, an anti-PD-L1 antibody binds specifically to PD-L1, and an anti-CTLA-4 antibody binds specifically to CTLA-4.

An “antigen-binding portion” of an antibody (also called an “antigen-binding fragment”) refers to one or more fragments of an antibody that retain the ability to bind specifically to the antigen bound by the whole antibody.

A “cancer” refers a broad group of various diseases characterized by the uncontrolled growth of abnormal cells in the body. Unregulated cell division and growth divide and grow results in the formation of malignant tumors that invade neighboring tissues and can also metastasize to distant parts of the body through the lymphatic system or bloodstream.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“Programmed Death-1” (PD-1) refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1” (PD-L1) is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

“Cytotoxic T-Lymphocyte Antigen-4” (CTLA-4) refers to an immunoinhibitory receptor belonging to the CD28 family. CTLA-4 is expressed exclusively on T cells in vivo, and binds to two ligands, CD80 and CD86 (also called B7-1 and B7-2, respectively). The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. AAB59385.

A “subject” includes any human or nonhuman animal. The term “nonhuman animal” includes, but is not limited to, vertebrates such as nonhuman primates, sheep, dogs, and rodents such as mice, rats and guinea pigs. In preferred embodiments, the subject is a human. The terms, “subject” and “patient” are used interchangeably herein.

The use of the term “flat dose” with regard to the methods and dosages of the disclosure means a dose that is administered to a patient without regard for the weight or body surface area (BSA) of the patient. The flat dose is therefore not provided as a mg/kg dose, but rather as an absolute amount of the agent (e.g., the anti-PD-1 antibody). For example, a 60 kg person and a 100 kg person would receive the same dose of an antibody (e.g., 240 mg of an anti-PD-1 antibody).

The use of the term “fixed dose” with regard to a method of the disclosure means that two or more different antibodies in a single composition (e.g., anti-PD-1 antibody and anti-CTLA-4 antibody or an anti-PD-L1 antibody and an anti-CTLA-4 antibody) are present in the composition in particular (fixed) ratios with each other. In some embodiments, the fixed dose is based on the weight (e.g., mg) of the antibodies. In certain embodiments, the fixed dose is based on the concentration (e.g., mg/ml) of the antibodies. In some embodiments, the ratio is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg first antibody (e.g., anti-PD-1 antibody or an anti-PD-L1 antibody) to mg second antibody (e.g., anti-CTLA-4 antibody). For example, the 3:1 ratio of an anti-PD-1 antibody and an anti-CTLA-4 antibody can mean that a vial can contain about 240 mg of the anti-PD-1 antibody and 80 mg of the anti-CTLA-4 antibody or about 3 mg/ml of the anti-PD-1 antibody and 1 mg/ml of the anti-CTLA-4 antibody.

The term “weight-based dose” as referred to herein means that a dose that is administered to a patient is calculated based on the weight of the patient. For example, when a patient with 60 kg body weight requires 3 mg/kg of an anti-PD-1 antibody, one can calculate and use the appropriate amount of the anti-PD-1 antibody (i.e., 180 mg) for administration.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” promotes cancer regression in a subject. In preferred embodiments, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, e.g., a tumor derived from an NSCLC, a therapeutically effective amount of an anti-cancer agent preferably inhibits cell growth or tumor growth by at least about 20%, more preferably by at least about 40%, even more preferably by at least about 60%, and still more preferably by at least about 80% relative to untreated subjects. In other preferred embodiments of the disclosure, tumor regression can be observed and continue for a period of at least about 20 days, more preferably at least about 40 days, or even more preferably at least about 60 days. Notwithstanding these ultimate measurements of therapeutic effectiveness, evaluation of immunotherapeutic drugs must also make allowance for immune-related response patterns.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

An “immunomodulator” or “immunoregulator” refers to an agent, e.g., an agent targeting a component of a signaling pathway that can be involved in modulating, regulating, or modifying an immune response. “Modulating,” “regulating,” or “modifying” an immune response refers to any alteration in a cell of the immune system or in the activity of such cell (e.g., an effector T cell, such as a Th1 cell). Such modulation includes stimulation or suppression of the immune system which can be manifested by an increase or decrease in the number of various cell types, an increase or decrease in the activity of these cells, or any other changes which can occur within the immune system. Both inhibitory and stimulatory immunomodulators have been identified, some of which can have enhanced function in a tumor microenvironment. In some embodiments, the immunomodulator targets a molecule on the surface of a T cell. An “immunomodulatory target” or “immunoregulatory target” is a molecule, e.g., a cell surface molecule, that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying the immune system or an immune response. In certain embodiments, the immunotherapy comprises administering an antibody to a subject. In other embodiments, the immunotherapy comprises administering a small molecule to a subject. In other embodiments, the immunotherapy comprises administering a cytokine or an analog, variant, or fragment thereof.

“Immuno stimulating therapy” or “immuno stimulatory therapy” refers to a therapy that results in increasing (inducing or enhancing) an immune response in a subject for, e.g., treating cancer.

“Potentiating an endogenous immune response” means increasing the effectiveness or potency of an existing immune response in a subject. This increase in effectiveness and potency can be achieved, for example, by overcoming mechanisms that suppress the endogenous host immune response or by stimulating mechanisms that enhance the endogenous host immune response.

A therapeutically effective amount of a drug includes a “prophylactically effective amount,” which is any amount of the drug that, when administered alone or in combination with an anti-neoplastic agent to a subject at risk of developing a cancer (e.g., a subject having a pre-malignant condition) or of suffering a recurrence of cancer, inhibits the development or recurrence of the cancer. In preferred embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

The term “tumor mutation burden” (TMB) as used herein refers to the number of somatic mutations in a tumor's genome and/or the number of somatic mutations per area of the tumor's genome. Germline (inherited) variants are excluded when determining TMB, because the immune system has a higher likelihood of recognizing these as self Tumor mutation burden (TMB) can also be used interchangeably with “tumor mutation load,” “tumor mutational burden,” or “tumor mutational load.”

TMB is a genetic analysis of a tumor's genome and, thus, can be measured by applying sequencing methods well known to those of skill in the art. The tumor DNA can be compared with DNA from patient-matched normal tissue to eliminate germline mutations or polymorphisms.

In some embodiments, TMB is determined by sequencing tumor DNA using a high-throughput sequence technique, e.g., next-generation sequencing (NGS) or an NGS-based method. In some embodiments, the NGS-based method is selected from whole genome sequencing (WGS), whole exome sequencing (WES), or comprehensive genomic profiling (CGP) of cancer gene panels such as FOUNDATIONONE CDX™ and MSK-IMPACT clinical tests. In some embodiments, TMB, as used herein, refers to the number of somatic mutations per megabase (Mb) of DNA sequenced. In one embodiment, TMB is measured using the total number of nonsynonymous mutations, e.g., missense mutation (i.e. changing a particular amino acid in the protein) and/or nonsense (causing premature termination and thus truncation of the protein sequence), identified by normalizing matched tumor with germline samples to exclude any inherited germline genetic alterations. In another embodiment, TMB is measured using the total number of missense mutations in a tumor. In order to measure TMB, a sufficient amount of sample is required. In one embodiment, tissue sample (for example, a minimum of 10 slides) is used for evaluation. In some embodiments, TMB is expressed as NsMs per megabase (NsM/Mb). 1 megabase represents 1 million bases.

The TMB status can be a numerical value or a relative value, e.g., high, medium, or low; within the highest fractile, or within the top tertile, of a reference set.

The term “high TMB” as used herein refers to a number of somatic mutations in a tumor's genome that is above a number of somatic mutations that is normal or average. In some embodiments, a TMB has a score of at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 445, at least 450, at least 455, at least 460, at least 465, at least 470, at least 475, at least 480, at least 485, at least 490, at least 495, or at least 500; in other embodiments a high TMB has a score of at least at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, or at least 250; and, in a particular embodiment, a high TMB has a score of at least 243.

In other embodiments, a “high TMB” refers to a TMB within the highest fractile of the reference TMB value. For example, all subject's with evaluable TMB data are grouped according to fractile distribution of TMB, i.e., subjects are rank ordered from highest to lowest number of genetic alterations and divided into a defined number of groups. In one embodiment, all subjects with evaluable TMB data are ranked ordered and divided into thirds, and a “high TMB” is within the top tertile of the reference TMB value. In a particular embodiment, the tertile boundaries are 0<100 genetic alterations; 100 to 243 genetic alterations; and >243 genetic alterations. It should be understood that, once rank ordered, subjects with evaluable TMB data can be divided into any number of groups, e.g., quartiles, quintiles, etc.

In some embodiments, a “high TMB” refers to a TMB of at least about 20 mutations/tumor, at least about 25 mutations/tumor, at least about 30 mutations/tumor, at least about 35 mutations/tumor, at least about 40 mutations/tumor, at least about 45 mutations/tumor, at least about 50 mutations/tumor, at least about 55 mutations/tumor, at least about 60 mutations/tumor, at least about 65 mutations/tumor, at least about 70 mutations/tumor, at least about 75 mutations/tumor, at least about 80 mutations/tumor, at least about 85 mutations/tumor, at least about 90 mutations/tumor, at least about 95 mutations/tumor, or at least about 100 mutations/tumor. In some embodiments, a “high TMB” refers to a TMB of at least about 105 mutations/tumor, at least about 110 mutations/tumor, at least about 115 mutations/tumor, at least about 120 mutations/tumor, at least about 125 mutations/tumor, at least about 130 mutations/tumor, at least about 135 mutations/tumor, at least about 140 mutations/tumor, at least about 145 mutations/tumor, at least about 150 mutations/tumor, at least about 175 mutations/tumor, or at least about 200 mutations/tumor. In certain embodiments, a tumor having a high TMB has at least about 100 mutations/tumor.

The “high TMB” can also be referred to as the number of mutations per megabase of tumor genome sequenced, e.g., as measured by a mutation assay, e.g., FOUNDATIONONE® CDX™ assay. In one embodiment, the high TMB refers to at least about 9, at least about 10, at least about 11, at least 12, at least about 13, at least about 14, at least about 15, at least about 16, at least about 17, at least about 18, at least about 19, or at least about 20 mutations per megabase of genome as measured by a FOUNDATIONONE® CDX™ assay. In a particular embodiment, the “high TMB” refers to at least 10 mutations per megabase of genome sequenced by a FOUNDATIONONE® CDX™ assay.

As used herein, the term “medium TMB” refers to a number of somatic mutations in a tumor's genome that is at or around a number of somatic mutations that is normal or average and the term “low TMB” refers to a number of somatic mutations in a tumor's genome that is below a number of somatic mutations that is normal or average. In a particular embodiment, a “high TMB” has a score of at least 243, a “medium TMB” has a score of between 100 and 242, and a “low TMB” has a score of less than 100 (or between 0 and 100). The “medium or low TMB” refers to less than 9 mutations per megabase of genome sequenced, e.g., as measured by a FOUNDATIONONE® CDX™ assay.

The term “reference TMB value” as referred to herein can be the TMB value shown in Table 9.

In some embodiments, TMB status can correlate with smoking status. In particular, subjects who currently or formerly smoke(d) often have more genetic alterations, e.g., missense mutations, than subjects who never smoke(d).

A tumor, e.g., a tumor derived from a NSCLC, with a high TMB can also have a high neoantigen load. As used herein, the term “neoantigen” refers to a newly formed antigen that has not been previously recognized by the immune system. A neoantigen can be a protein or peptide that is recognized as foreign (or non-self) by the immune system. Transcription of a gene in the tumor genome harboring a somatic mutation results in mutated mRNA that, when translated, gives rise to a mutated protein, which is then processed and transported to the ER lumen and binds to MHC class I complex, facilitating T-cell recognition of the neoantigen. Neoantigen recognition can promote T-cell activation, clonal expansion, and differentiation into effector and memory T-cells. Neoantigen load can correlate with TMB. In some embodiments, TMB is assessed as a surrogate for measuring tumor neoantigen load. The TMB status of a tumor, e.g., a tumor derived from a NSCLC, can be used as a factor, alone or in combination with other factors, in determining whether a patient is likely to benefit from a particular anti-cancer agent or type of treatment or therapy, e.g., a combination therapy comprising (a) an anti-PD-1 antibody or an anti-PD-L1 antibody and (b) an anti-CTLA-4 antibody. In one embodiment, a high TMB status (or a high TMB) indicates an enhanced likelihood of benefit from immuno-oncology and, thus, can be used to identify patients more likely to benefit from therapy of a combination therapy comprising (a) an anti-PD-1 antibody or an anti-PD-L1 antibody and (b) an anti-CTLA-4 antibody. Similarly, tumors with high tumor neoantigen load and high TMB are more likely to be immunogenic than tumors with low neoantigen load and low TMB. In addition, high-neoantigen/high-TMB tumors are more likely to be recognized as non-self by the immune system, thus triggering an immune-mediated antitumor response. In one embodiment, a high TMB status and a high neoantigen load indicate an enhanced likelihood of benefit from immuno-oncology, e.g., a combination therapy comprising (a) an anti-PD-1 antibody or an anti-PD-L1 antibody and (b) an anti-CTLA-4 antibody. As used herein, the term “benefit from therapy” refers to an improvement in one or more of overall survival, progression-free survival, partial response, complete response, and overall response rate and can also include a reduction in tumor growth or size, a decrease in severity of disease symptoms, an increase in frequency and duration of disease symptom-free periods, or a prevention of impairment or disability due to the disease affliction.

Other factors, e.g., environmental factors, can associate with TMB status. For example, smoking status of patients with NSCLC was correlated with TMB distribution, whereby current and former smokers had higher median TMB compared with those patients who had never smoked. See Peters et al., AACR, Apr. 1-5, 2017, Washington, D.C. The presence of a driver mutation in NSCLC tumors was associated with younger age, female sex, and non-smoker status. See Singal et al., ASCO, Jun. 1-5, 2017; Chicago, IL. A trend associating the presence of driver mutations, such as EGFR, ALK, or KRAS, with lower TMB was observed (P=0.06). Davis et al., AACR, Apr. 1-5, 2017, Washington, D.C.

The term “somatic mutation” as used herein refers to an acquired alteration in DNA that occurs after conception. Somatic mutations can occur in any of the cells of the body except the germ cells (sperm and egg) and therefore are not passed on to children. These alterations can, but do not always, cause cancer or other diseases. The term “germline mutation” refers to a gene change in a body's reproductive cell (egg or sperm) that becomes incorporated into the DNA of every cell in the body of the offspring. Germline mutations are passed on from parents to offspring. Also called a “hereditary mutation.” In the analysis of TMB, germline mutations are considered as a “baseline,” and are subtracted from the number of mutations found in the tumor biopsy to determine the TMB within the tumor. As germline mutations are found in every cell in the body, their presence can be determined via less invasive sample collections than tumor biopsies, such as blood or saliva. Germline mutations can increase the risk of developing certain cancers, and can play a role in the response to chemotherapy.

The term “measuring” or “measured” or “measurement” when referring to TMB status means determining a measurable quantity of somatic mutations in a biological sample of the subject. It will be appreciated that measuring can be performed by sequencing nucleic acids, e.g., cDNA, mRNA, exoRNA, ctDNA, and cfDNA, in the sample. The measuring is performed on a subject's sample and/or a reference sample or samples and can, for example, be detected de novo or correspond to a previous determination. The measuring can be performed, for example, using PCR methods, qPCR methods, Sanger sequencing methods, genomic profiling methods (including comprehensive gene panels), exome sequencing methods, genome sequencing methods, and/or any other method disclosed herein, as is known to a person of skill in the art. In some embodiments, the measuring identifies a genomic alteration in the sequenced nucleic acids. The genomic (or gene) profiling methods can involve panels of a predetermined set of genes, e.g., 150-500 genes, and in some instances the genomic alterations evaluated in the panel of genes are correlated with total somatic mutations evaluated. As used herein when referring to sequencing, the term “gene” includes DNA coding regions (e.g., exons), DNA non-coding regions associated with a coding region (e.g., introns and promoters), and mRNA transcripts.

The term “genomic alteration” as used herein refers to a change (or mutation) in the nucleotide sequence of the genome of a tumor, which change is not present in the germline nucleotide sequence, and which in some embodiments is a nonsynonymous mutation including, but not limited to, a base pair substitution, a base pair insertion, a base pair deletion, a copy number alteration (CNA), a gene rearrangement, and any combination thereof. In a particular embodiment, the genomic alterations measured in the biological sample are missense mutations.

The term “whole genome sequencing” or “WGS,” as used herein, refers to a method of sequencing the entire genome. The term “whole exome sequencing” or “WES,” as used herein, refers to a method of sequencing all the protein-coding regions (exons) of the genome.

A “cancer gene panel,” “hereditary cancer panel,” “comprehensive cancer panel,” or “multigene cancer panel,” as used herein, refers to a method of sequencing a subset of targeted cancer genes, including coding regions, introns, promoters, and/or mRNA transcripts. In some embodiments, the CGP comprises sequencing at least about 15, at least about 20, at least about 25, at least about 30, at least about 35, at least about 40, at least about 45, or at least about 50 targeted cancer genes.

The term “genomic profiling assay,” “comprehensive genomic profiling,” or “CGP” refers to an assay that analyzes a panel of genes and select introns for in vitro diagnosis. CGP is a combination of NGS and targeted bioinformatics analysis to screen for mutations in known clinically relevant cancer genes. This method can be used to catch mutations that are missed by testing “hotspots” (e.g., BRCA1/BRCA2 mutations or microsatellite markers). In some embodiments, the CGP further includes one or more mRNA transcript, non-coding RNA, and/or promoter region. In one embodiment, the genes in the panel are cancer-related genes. In another embodiment, a genomic profiling assay is a FOUNDATIONONE® assay.

The term “harmonization” refers to a study conducted to determine the comparability between two or more measures and/or diagnostic tests. Harmonization studies provide a systematic approach to address questions of how diagnostic tests compare with each other, as well as their interchangeability when used to determine the biomarker status of a patient's tumor. In general, at least one well-characterized measure and/or diagnostic test is used as a standard for comparison with others. Concordance assessment is often utilized in harmonization studies.

The term “concordance,” as used herein, refers to a degree of agreement between two measurements and/or diagnostic tests. Concordance can be established using both qualitative and quantitative methods. Quantitative methods to assess concordance differ based on the type of measurement. A particular measurement can be expressed either as 1) a categorical/dichotomized variable or 2) a continuous variable. A “categorical/dichotomized variable” (e.g., above or below TMB cut-off) may use percent agreements, such as overall percent agreement (OPA), positive percent agreement (PPA), or negative percent agreement (NPA), to assess concordance. A “continuous variable” (e.g., TMB by WES) uses Spearman's rank correlation or Pearson's correlation coefficient (r), which takes on values −1≤r≤+1, to assess concordance across a spectrum of values (Note r=+1 or −1 means that each of the variables is perfectly correlated). The term “analytical concordance” refers to the degree of agreement in the performance (e.g., identification of biomarkers, genomic alteration types, and genomic signatures, and assessment of test reproducibility) of two assays or diagnostic tests to support clinical use. The term “clinical concordance” refers to the degree of agreement in how the two assays or diagnostic tests correlate with clinical outcome.

The term “microsatellite instability” or “MSI” refers to a change that occurs in the DNA of certain cells (such as tumor cells) in which the number of repeats of microsatellites (short, repeated sequences of DNA) is different than the number of repeats that was in the DNA when it was inherited. MSI can be high microsatellite instability (MSI-H) or low microsatellite instability (MSI-L). Microsatellites are short tandem DNA repeat sequences of 1-6 bases. These are prone to DNA replication errors, which are repaired by mismatch repair (MMR). Hence microsatellites are good indicators of genome instability, especially deficient mismatch repair (dMMR). MSI is usually diagnosed by screening 5 microsatellite markers (BAT-25, BAT-26, NR21, NR24, and NR27). MSI-H represents the presence of at least 2 unstable markers among 5 microsatellite markers analyzed (or ≥30% of the markers if a larger panel is used). MSI-L means instability of 1 MSI marker (or 10%-30% of markers in larger panels). MSS means the absence of an unstable microsatellite marker.

The term “biological sample” as used herein refers to biological material isolated from a subject. The biological sample can contain any biological material suitable for determining TMB, for example, by sequencing nucleic acids in the tumor (or circulating tumor cells) and identifying a genomic alteration in the sequenced nucleic acids. The biological sample can be any suitable biological tissue or fluid such as, for example, tumor tissue, blood, blood plasma, and serum. In one embodiment, the sample is a tumor tissue biopsy, e.g., a formalin-fixed, paraffin-embedded (FFPE) tumor tissue or a fresh-frozen tumor tissue or the like. In another embodiment, the biological sample is a liquid biopsy that, in some embodiments, comprises one or more of blood, serum, plasma, circulating tumor cells, exoRNA, ctDNA, and cfDNA.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±three days, i.e., every eleven days to every seventeen days. Similar approximations apply, for example, to once about every three weeks, once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 100. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

A list of abbreviations is provided in Table 1.

TABLE 1
List of Abbreviations

| Term | Definition |
|---|---|
| Ab | antibody |
| AE | adverse event |
| ALK | anaplastic lymphoma kinase |
| AUC | area under the concentration-time curve |
| BICR | blinded independent central review |
| BMS | Bristol-Myers Squibb |
| BSA | body surface area |
| cfDNA | cell-free DNA |
| CI | confidence interval |
| CNS | central nervous system |
| CONSORT | consolidated standards of reporting trials |
| CR | complete response |
| ctDNA | circulating tumor DNA |
| CTLA-4 | cytotoxic T-lymphocyte-associated protein 4 |
| ECOG | Eastern Cooperative Oncology Group |
| e.g. | exempli gratia (for example) |
| EGFR | epidermal growth factor receptor |
| ELISA | enzyme-linked immunosorbent assay |
| exoRNA | exosomal RNA |
| HuMab | human antibody; human monoclonal antibody |
| i.e. | id est (that is) |
| IV | Intravenous |
| Kg | kilogram |
| mAb | monoclonal antibody |
| MB | megabase |
| mg | milligram |
| MO | month |
| N | number of subjects or observations |
| NCCN | National Comprehensive Cancer Network |
| NSCLC | non-small cell lung cancer |
| ORR | overall response rate |
| OS | overall survival |
| PD-1 | programmed death-1 |
| PD-L1 | programmed death-ligand 1 |
| PD-L2 | programmed death-ligand 2 |
| PFS | progression-free survival |
| PR | partial response |
| Q2W | once every two weeks |
| Q6W | once every six weeks |
| Q12W | once every twelve weeks |
| RECIST | response evaluation criteria in solid tumors |
| TILs | tumor infiltrating lymphocytes |
| TMB | tumor mutation burden |
| WES | whole exome sequencing |
| WGS | whole genome sequencing |

Various aspects of the disclosure are described in further detail in the following subsections.

### Methods of the Disclosure

Certain aspects of the present disclosure are directed to methods for treating a subject afflicted with a tumor derived from a NSCLC having a high TMB status, comprising administering to the subject a therapeutically effective amount of (a) an anti-PD-1 antibody or an anti-PD-L1 antibody and (b) an anti-CTLA-4 antibody. Other aspects of the present disclosure are directed to a method for identifying a subject who is afflicted with a tumor derived from a NSCLC and is suitable for a combination therapy of (a) an anti-PD-1 antibody or an anti-PD-L1 antibody and (b) an anti-CLTA-4 antibody, comprising measuring a TMB status of a biological sample of the subject, wherein the TMB status comprises at least about 10 mutations per megabase of genome examined and wherein the subject is identified as being suitable for the combination therapy. The disclosure is based on the fact that tumor immunogenicity is directly related to TMB and/or neoantigen load.

As a tumor grows, it accumulates somatic mutations not present in germline DNA. TMB refers to the number of somatic mutations in a tumor's genome and/or the number of somatic mutations per area of the tumor genome (after taking into account germline variant DNA). The acquisition of somatic mutations and, thus, a higher TMB can be influenced by distinct mechanisms, such as exogenous mutagen exposure (e.g., tobacco smoking) and DNA mismatch repair mutations (e.g., MSI in colorectal and esophageal cancers). In solid tumors, about 95% of mutations are single-base substitutions. (Vogelstein et al., Science (2013) 339:1546-1558.) A “nonsynonymous mutation” herein refers to a nucleotide mutation that alters the amino acid sequence of a protein. Missense mutations and nonsense mutations can be both nonsynonymous mutations. A “missense mutation” herein refers to a nonsynonymous point mutation in which a single nucleotide change results in a codon that codes for a different amino acid. A “nonsense mutation” herein refers to a nonsynonymous point mutation in which a codon is changed to a premature stop codon that leads to truncation of the resulting protein.

In one embodiment, somatic mutations can be expressed at the RNA and/or protein level, resulting in neoantigens (also referred to as neoepitopes). Neoantigens can influence an immune-mediated anti-tumor response. For example, neoantigen recognition can promote T-cell activation, clonal expansion, and differentiation into effector and memory T-cells.

As a tumor develops, early clonal mutations (or “trunk mutations”) can be carried by most or all tumor cells, while late mutations (or “branch mutations”) can occur in only a subset of tumor cells or regions. (Yap et al., Sci Tranl Med (2012) 4:1-5; Jamai-Hanjani et al., (2015) Clin Cancer Res 21:1258-1266.) As a result, neoantigens derived from clonal “trunk” mutations are more widespread in the tumor genome than “branch” mutations and, thus, can lead to a high number of T cells reactive against the clonal neoantigen. (McGranahan et al., (2016) 351:1463-1469.) Generally, tumors with a high TMB can also have a high neoantigen load, which can lead to high tumor immunogenicity and increased T-cell reactivity and anti-tumor response. As such, cancers with a high TMB can respond well to treatment with immunotherapies, e.g., an anti-PD-1 antibody or anti-PD-L1 antibody.

Advances in sequencing technologies allow for evaluation of the tumor's genomic mutation landscape. Any sequencing methods known to those of skill in the art can be used to sequence nucleic acids from the tumor genome (e.g., obtained from a biological sample from a subject afflicted with a tumor). In one embodiment, PCR or qPCR methods, Sanger sequencing methods, or next-generation sequencing (“NGS”) methods (such as genomic profiling, exome sequencing, or genome sequencing) can be used to measure TMB. In some embodiments, the TMB status is measured using genomic profiling. Genomic profiling involves analyzing nucleic acids from tumor samples, including coding and non-coding regions, and can be performed using methods having integrated optimized nucleic acid selection, read alignment, and mutation calling. In some embodiments, gene profiling provides next generation sequencing (NGS)-based analysis of tumors that can be optimized on a cancer-by-cancer, gene-by-gene, and/or site-by-site basis. Genome profiling can integrate the use of multiple, individually tuned, alignment methods or algorithms to optimize performance in sequencing methods, particularly in methods that rely on massively parallel sequencing of a large number of diverse genetic events in a large number of diverse genes. Genomic profiling provides for a comprehensive analysis of a subject's cancer genome, with clinical grade quality, and the output of the genetic analysis can be contextualized with relevant scientific and medical knowledge to increase the quality and efficiency of cancer therapy.

Genomic profiling involves a panel of a predefined set of genes comprising as few as five genes or as many as 1000 genes, about 25 genes to about 750 genes, about 100 genes to about 800 genes, about 150 genes to about 500 genes, about 200 genes to about 400 genes, about 250 genes to about 350 genes. In one embodiment, the genomic profile comprises at least 300 genes, at least 305 genes, at least 310 genes, at least 315 genes, at least 320 genes, at least 325 genes, at least 330 genes, at least 335 genes, at least 340 genes, at least 345 genes, at least 350 genes, at least 355 genes, at least 360 genes, at least 365 genes, at least 370 genes, at least 375 genes, at least 380 genes, at least 385 genes, at least 390 genes, at least 395 genes, or at least 400 genes. In another embodiment, the genomic profile comprises at least 325 genes. In a particular embodiment, the genomic profile comprises at least 315 cancer-related genes and introns in 28 genes (FOUNDATIONONE®) or the complete DNA coding sequence of 406 genes, introns in 31 genes with rearrangements, and the RNA sequence (cDNA) of 265 genes (FOUNDATIONONE® Heme). In another embodiment, the genomic profile comprises 26 genes and 1000 associated mutations (EXODX® Solid Tumor). In yet another embodiment, the genomic profile comprises 76 genes (Guardant360). In yet another embodiment, the genomic profile comprises 73 genes (Guardant360). In another embodiment, the genomic profile comprises 354 genes and introns in 28 genes for rearrangements (FOUNDATIONONE® CDX™). In certain embodiments, the genomic profile is FOUNDATIONONE® F1CDx. In another embodiment, the genomic profile comprises 468 genes (MSK-IMPACT™). One or more genes can be added to the genome profile as more genes are identified to be related to oncology.

### FOUNDATIONONE® Assay

The FOUNDATIONONE® assay is comprehensive genomic profiling assay for solid tumors, including but not limited to solid tumors of the lung, colon, and breast, melanoma, and ovarian cancer. The FOUNDATIONONE® assay uses a hybrid-capture, next-generation sequencing test to identify genomic alterations (base substitutions, insertions and deletions, copy number alterations, and rearrangements) and select genomic signatures (e.g., TMB and microsatellite instability). The assay covers 322 unique genes, including the entire coding region of 315 cancer-related genes, and selected introns from 28 genes. The full list of FOUNDATIONONE® assay genes is provided in Tables 2 and 3. See FOUNDATIONONE: Technical Specifications, Foundation Medicine, Inc., available at FoundationMedicine.com, last visited Mar. 16, 2018, which is incorporated by reference herein in its entirety.

TABLE 2
List of genes wherein entire coding sequences are assayed in the FOUNDATIONONE ® assay.

| | | | | | | | | | |
|---|---|---|---|---|---|---|---|---|---|
| ABL1 | BRAF | CHEK1 | FANCC | GATA3 | JAK2 | MITF | PDCD1LG2 | RBM10 | STAT4 |
| | | | | | | | (PD-L2) | | |
| ABL2 | BRCA1 | CHEK2 | FANCD2 | GATA4 | JAK3 | MLH1 | PDGFRA | RET | STK11 |
| ACVR1B | BRCA2 | CIC | FANCE | GATA6 | JUN | MPL | PDGFRB | RICTOR | SUFU |
| AKT1 | BRD4 | CREBBP | FANCF | GID4 | KAT6A | MRE11A | PDK1 | RNF43 | SYK |
| | | | | (C17orf39) | (MYST3) | | | | |
| AKT2 | BRIP1 | CRKL | FANCG | GLl1 | KDM5A | MSH2 | PIK3C2B | ROS1 | TAF1 |
| AKT3 | BTG1 | CRLF2 | FANCL | GNA11 | KDM5C | MSH6 | PIK3CA | RPTOR | TBX3 |
| ALK | BTK | CSF1R | FAS | GNA13 | KDM6A | MTOR | PIK3CB | RUNX1 | TERC |
| AMER1 | C11orf30 | CTCF | FAT1 | GNAQ | KDR | MUTYH | PIK3CG | RUNXIT1 | TERT |
| (FAM123B) | (EMSY) | | | | | | | | (Promoter |
| | | | | | | | | | only) |
| APC | CARD11 | CTNNA1 | FBXW7 | GNAS | KEAP1 | MYC | PIK3R1 | SDHA | TET2 |
| AR | CBFB | CTNNB1 | FGF10 | GPR124 | KEL | MYCL | PIK3R2 | SDHB | TGFBR2 |
| | | | | | | (MYCL1) | | | |
| ARAF | CBL | CUL3 | FGF14 | GRIN2A | KIT | MYCN | PLCG2 | SDHC | TNFAIP3 |
| ARFRP1 | CCND1 | CYLD | FGF19 | GRM3 | KLHL6 | MYD88 | PMS2 | SDHD | TNFRSF14 |
| ARID1A | CCND2 | DAXX | FGF23 | GSK3B | KMT2A | NF1 | POLD1 | SETD2 | TOP1 |
| | | | | | (MLL) | | | | |
| ARID1B | CCND3 | DDR2 | FGF3 | H3F3A | KMT2C | NF2 | POLE | SF3B1 | TOP2A |
| | | | | | (MLL3) | | | | |
| ARID2 | CCNE1 | DICER1 | FGF4 | HGF | KMT2D | NFE2L2 | PPP2R1A | SLIT2 | TP53 |
| | | | | | (MLL2) | | | | |
| ASXL1 | CD274 | DNMT3A | FGF6 | HNFlA | KRAS | NFKBIA | PRDM1 | SMAD2 | TSC1 |
| | (PD-L1) | | | | | | | | |
| ATM | CD79A | DOT1L | FGFR1 | HRAS | LMO1 | NKX2-1 | PREX2 | SMAD3 | TSC2 |
| ATR | CD79B | EGFR | FGFR2 | HSD3B1 | LRP1B | NOTCH1 | PRKARIA | SMAD4 | TSHR |
| ATRX | CDC73 | EP300 | FGFR3 | HSP90AA1 | LYN | NOTCH2 | PRKCI | SMARCA4 | U2AF1 |
| AURKA | CDH1 | EPHA3 | FGFR4 | IDH1 | LZTR1 | NOTCH3 | PRKDC | SMARCB1 | VEGFA |
| AURKB | CDK12 | EPHA5 | FH | IDH2 | MAGI2 | NPM1 | PRSS8 | SMO | VHL |
| AXIN1 | CDK4 | EPHA7 | FLCN | IGF1R | MAP2K1 | NRAS | PTCH1 | SNCAIP | WISP3 |
| | | | | | (MEK1) | | | | |
| AXL | CDK6 | EPHB1 | FLT1 | IGF2 | MAP2K2 | NSD1 | PTEN | SOCS1 | WT1 |
| | | | | | (MEK2) | | | | |
| BAP1 | CDK8 | ERBB2 | FLT3 | IKBKE | MAP2K4 | NTRK1 | PTPN11 | SOX10 | XPO1 |
| BARD1 | CDKN1A | ERBB3 | FLT4 | IKZF1 | MAP3K1 | NTRK2 | QKI | SOX2 | ZBTB2 |
| BCL2 | CDKN1B | ERBB4 | FOXL2 | IL7R | MCL1 | NTRK3 | RAC1 | SOX9 | ZNF217 |
| BCL2L1 | CDKN2A | ERG | FOXP1 | INHBA | MDM2 | NUP93 | RAD50 | SPEN | ZNF703 |
| BCL2L2 | CDKN2B | ERRFl1 | FRS2 | INPP4B | MDM4 | PAK3 | RAD51 | SPOP | |
| BCL6 | CDKN2C | ESR1 | FUBP1 | IRF2 | MED12 | PALB2 | RAF1 | SPTA1 | |
| BCOR | CEBPA | EZH2 | GABRA6 | IRF4 | MEF2B | PARK2 | RANBP2 | SRC | |
| BCORL1 | CHD2 | FAM46C | GATA1 | IRS2 | MEN1 | PAX5 | RARA | STAG2 | |
| BLM | CHD4 | FANCA | GATA2 | JAK1 | MET | PBRM1 | RB1 | STAT3 | |

TABLE 3
List of genes wherein selected introns are assayed in the FOUNDATIONONE ® assay.

| | | | | | | |
|---|---|---|---|---|---|---|
| ALK | BRCA1 | ETV1 | FGFR1 | MSH2 | NTRK1 | RARA |
| BCL2 | BRCA2 | ETV4 | FGFR2 | MYB | NTRK2 | RET |
| BCR | BRD4 | ETV5 | FGFR3 | MYC | PDGFRA | ROS1 |
| BRAF | EGFR | ETV6 | KIT | NOTCH2 | RAF1 | TMPRSS2 |

### EXODX® Solid Tumor Assay

In one embodiment, TB is measured using the EXODX® Solid Tumor assay. The EXODX® Solid Tumor assay is an exoRNA- and cfDNA-based assay, which detects actionable mutations in cancer pathways. The EXODX® Solid Tumor assay is a plasma-based assay that does not require a tissue sample. The EXODX® Solid Tumor assay covers 26 genes and 1000 mutations. The specific genes covered by the EXODX® Solid Tumor assay are shown in Table 4. See Plasma-Based Solid Tumor Mutation Panel Liquid Biopsy, Exosome Diagnostics, Inc., available at exosomedx.com, last accessed on Mar. 25, 2019.

TABLE 4
Genes covered by the EXODX ® Solid Tumor assay.

| | | | | | | | | |
|---|---|---|---|---|---|---|---|---|
| BRAF | MEK1 | KIT | ROS1 | ALK | PTEN | TP53 | FGFR3 | TSC2 |
| NRAS | KRAS | PDGFRA | RET | AKT1 | DH2 | NOTCH1 | NTRK1 | CDKN2A |
| PIK3CA | EGFR | EML4- | HER- | ARv7 | mTOR | Hedgehog | TSC1 | |
| | | ALK | 2/NEU; | | | | | |
| | | | ERBB2 | | | | | |

### Guardant360 Assay

In some embodiments, TMB status is determined using the Guardant360 assay. The Guardant360 assay measures mutations in at least 73 genes (Table 5), 23 indels (Table 6), 18 CNVs (Table 7), and 6 fusion genes (Table 8). See GuardantHealth.com, last accessed on Mar. 25, 2019.

TABLE 5
Guardant360 assay genes.

| | | | | | | |
|---|---|---|---|---|---|---|
| AKT1 | CCND2 | EZH2 | IDH1 | MLH1 | PDGFRA | SMAD4 |
| ALK | CCNE1 | FBXW7 | IDH2 | MPL | PIK3CA | SMO |
| APC | CDH1 | FGFR1 | JAK2 | MTOR | PTEN | STK11 |
| AR | CDK4 | FGFR2 | JAK3 | MYC | PTPN11 | TERT |
| | | | | | | (including promoter) |
| ARAF | CDK6 | FGFR3 | KIT | NF1 | RAF1 | TP53 |
| ARID1A | CDKN2A | GATA3 | KRAS | NFE2L2 | RB1 | TSC1 |
| ATM | CTNNB1 | GNA11 | MAP2K1 | NOTCH1 | RET | VHL |
| BRAF | DDR2 | GNAQ | MAP2K2 | NPM1 | RHEB | |
| BRCA1 | EGFR | GNAS | MAPK1 | NRAS | RHOA | |
| BRCA2 | ERBB2 | HNF1A | MAPK3 | NTRK1 | RIT1 | |
| CCND1 | ESR1 | HRAS | MET | NTRK3 | ROS1 | |

TABLE 6
Guardant360 assay indels

| | | | | | | | |
|---|---|---|---|---|---|---|---|
| APC | BRCA1 | CDKN2A | GATA3 | MLH1 | PDGFRA | SMAD4 | TSC1 |
| ARID1A | BRCA2 | EGFR | KIT | MTOR | PTEN | STK11 | VHL |
| ATM | CDH1 | ERBB2 | MET | NF1 | RB1 | TP53 | |

TABLE 7
Guardant360 assay amplifications (CNVs).

| | | | | | |
|---|---|---|---|---|---|
| AR | CCND2 | CDK6 | FGFR1 | KRAS | PDGFRA |
| BRAF | CCNE1 | EGFR | FGFR2 | MET | PIK3CA |
| CCND1 | CDK4 | ERBB2 | KIT | MYC | RAF1 |

TABLE 8
Guardant360 assay fusions

| | | |
|---|---|---|
| ALK | FGFR3 | RET |
| FGFR2 | NTRK1 | ROS1 |

### ILLUMINA® TruSight Assay

In some embodiments, TMB is determined using the TruSight Tumor 170 assay (ILLUMINA). The TruSight Tumor 170 assay is a next-generation sequencing assay that covers 170 genes associated with common solid tumors, which simultaneously analyzes DNA and RNA. The TruSight Tumor 170 assay assesses fusions, splice variants, insertions/deletions, single nucleotide variants (SNVs), and amplifications. The TruSight Tumor 170 assay gene lists are shown in Tables 12-14.

TABLE 9
TruSight Tumor 170 assay genes (amplifications).

| | | | | | |
|---|---|---|---|---|---|
| AKT2 | CDK4 | FGF1 | FGF7 | LAMP | PDGFRB |
| ALK | CDK6 | FGF10 | FGF8 | MDAM2 | PIK3CA |
| AR | CHEK1 | FGF14 | FGF9 | MDM4 | PIK3CB |
| ATM | CHEK2 | FGF19 | FGFR1 | MET | PTEN |
| BRAF | EGFR | FGF2 | FGFR2 | MYC | RAF1 |
| BRCA1 | ERBB2 | FGF23 | FGFR3 | MYCL1 | RET |
| BRCA2 | ERBB3 | FGF3 | FGFR4 | MYCN | RICTOR |
| CCND1 | ERCC1 | FGF4 | JAK2 | NRAS | RPS6KB1 |
| CCND3 | ERCC2 | FGF5 | KIT | NRG1 | TFRC |
| CCNE1 | ESR1 | FGF6 | KRAS | PDGFRA | |

TABLE 10
TruSight Tumor 170 assay genes (fusions).

| | | | | | | | |
|---|---|---|---|---|---|---|---|
| ABL1 | BRCA1 | ERG | FGFR1 | JAK2 | MSH2 | NTRK2 | PPARG |
| AKT3 | BRCA2 | ESR1 | FGFR2 | KDR | MYC | NTRK3 | RAF1 |
| ALK | CDK4 | ETS1 | FGFR3 | KIF5B | NOTCH1 | PAX3 | RET |
| AR | CSF1R | ETV1 | FGFR4 | KIT | NOTCH2 | PAX7 | ROS1 |
| AXL | EGFR | ETV4 | FLI1 | KMT2A | NOTCH3 | PDGFRA | RPS6KB1 |
| | | | | (MLL) | | | |
| BCL2 | EML4 | ETV5 | FLT1 | MET | NRG1 | PDGFRB | TMPRSS2 |
| BRAF | ERBB2 | EWSR1 | FLT3 | MLLT3 | NTRK1 | PIK3CA | |

TABLE 11
TruSight Tumor 170 assay genes (small variants).

| | | | | | | | | | |
|---|---|---|---|---|---|---|---|---|---|
| AKT1 | BRCA2 | CHEK1 | ESR1 | FGF7 | HRAS | MET | NF1 | PMS2 | SLX4 |
| AKT2 | BRIP1 | CHEK2 | EZH2 | FGF8 | IDH1 | MLH1 | NOTCH1 | PPP2R2A | SMAD4 |
| AKT3 | BTK | CREBBP | FAM175A | FGF9 | IDH2 | MLLT3 | NOTCH2 | PTCH1 | SMARCB1 |
| ALK | CARD11 | CSF1R | FANCI | FGFR1 | INPP4B | MPL | NOTCH3 | PTEN | SMO |
| APC | CCND1 | CTNNB1 | FANCL | FGFR2 | JAK2 | MRE11A | NPM1 | PTPN11 | SRC |
| AR | CCND2 | DDR2 | FBXW7 | FGFR3 | JAK3 | MSH2 | NRAS | RAD51 | STK11 |
| ARID1A | CCNE1 | DNMT3A | FGF1 | FGFR4 | KDR | MSH3 | NRG1 | RAD51B | TERT |
| ATM | CD79A | EGFR | FGF10 | FLT1 | KIT | MSH6 | PALB2 | RAD51C | TET2 |
| ATR | CD79B | EP300 | FGF14 | FLT3 | KMT2A | MTOR | PDGFRA | RAD51D | TP53 |
| | | | | | (MLL) | | | | |
| BAP1 | CDH1 | ERBB2 | FGF2 | FOXL2 | KRAS | MUTYH | PDGFRB | RAD54L | TSC1 |
| BARD1 | CDK12 | ERBB3 | FGF23 | GEN1 | MAP2K1 | MYC | PIK3CA | RB1 | TSC2 |
| BCL2 | CDK4 | ERBB4 | FGF3 | GNA11 | MAP2K2 | MYCL1 | PIK3CB | RET | VHL |
| BCL6 | CDK6 | ERCC1 | FGF4 | GNAQ | MCL1 | MYCN | PIK3CD | RICTOR | XRCC2 |
| BRAF | CDKN2A | ERCC2 | FGF5 | GNAS | MDM2 | MYD88 | PIK3CG | ROS1 | |
| BRCA1 | CEBPA | ERG | FGF6 | HNFlA | MDM4 | NBN | PIK3R1 | RPS6KB1 | |

### FOUNDATIONONE® F1CDx Assay

FOUNDATIONONE® CDX™ (“F1CDx”) is a next generation sequencing based in vitro diagnostic device for detection of substitutions, insertion and deletion alterations (indels), and copy number alterations (CNAs) in 324 genes and select gene rearrangements, as well as genomic signatures including microsatellite instability (MSI) and tumor mutation burden (TMB) using DNA isolated from formalin-fixed paraffin embedded (FFPE) tumor tissue specimens. F1CDx is approved by the United States Food and Drug Administration (FDA) for several tumor indications, including NSCLC, melanoma, breast cancer, colorectal cancer, and ovarian cancer.

The F1CDx assay employs a single DNA extraction method from routine FFPE biopsy or surgical resection specimens, 50-1000 ng of which will undergo whole-genome shotgun library construction and hybridization-based capture of all coding exons from 309 cancer-related genes, one promoter region, one non-coding (ncRNA), and selected intronic regions from 34 commonly rearranged genes, 21 of which also include the coding exons. Tables 12 and 13 provide the complete list of genes included in F1CDx. In total, the assay detects alterations in a total of 324 genes. Using the ILLUMINA® HiSeq 4000 platform, hybrid capture-selected libraries are sequenced to high uniform depth (targeting >500× median coverage with >99% of exons at coverage >100×). Sequence data is then processed using a customized analysis pipeline designed to detect all classes of genomic alterations, including base substitutions, indels, copy number alterations (amplifications and homozygous gene deletions), and selected genomic rearrangements (e.g., gene fusions). Additionally, genomic signatures including microsatellite instability (MSI) and tumor mutation burden (TMB) are reported.

TABLE 12
Genes with full coding exonic regions included in FOUNDATIONONE ® CDX ™ for the
detection of substitutions, insertions and deletions (indels), and copy number alterations (CNAs).

| | | | | | | | | | |
|---|---|---|---|---|---|---|---|---|---|
| ABL1 | BRCA2 | CDKN2C | ERCC4 | GATA3 | KDM5C | MRE11A | PARP2 | RAD51 | SOX9 |
| ACVR1B | BRD4 | CEBPA | ERG | GATA4 | KDM6A | MSH2 | PARP3 | RAD51B | SPEN |
| AKT1 | BRIP1 | CHEK1 | ERRFI1 | GATA6 | KDR | MSH3 | PAX5 | RAD51C | SPOP |
| AKT2 | BTG1 | CHEK2 | ESR1 | GID4 | KEAP1 | MSH6 | PBRM1 | RAD51D | SRC |
| | | | | (C17orf39) | | | | | |
| AKT3 | BTG2 | CIC | EZH2 | GNA11 | KEL | MST1R | PDCD1 | RAD52 | STAG2 |
| ALK | BTK | CREBBP | FAM46C | GNA13 | KIT | MTAP | PDCD1LG2 | RAD54L | STAT3 |
| ALOX12B | C11orf30 | CRKL | FANCA | GNAQ | KLHL6 | MTOR | PDGFRA | RAF1 | STK11 |
| AMER1 | CALR | CSF1R | FANCC | GNAS | KMT2A | MUTYH | PDGFRB | RARA | SUFU |
| | | | | | (MLL) | | | | |
| APC | CARD11 | CSF3R | FANCG | GRM3 | KMT2D | MYC | PDK1 | RB1 | SYK |
| | | | | | (MLL2) | | | | |
| AR | CASP8 | CTCF | FANCL | GSK3B | KRAS | MYCL | PIK3C2B | RBM10 | TBX3 |
| ARAF | CBFB | CTNNA1 | FAS | H3F3A | LTK | MYCN | PIK3C2G | REL | TEK |
| ARFRP1 | CBL | CTNNB1 | FBXW7 | HDAC1 | LYN | MYD88 | PIK3CA | RET | TET2 |
| ARID1A | CCND1 | CUL3 | FGF10 | HGF | MAF | NBN | PIK3CB | RICTOR | TGFBR2 |
| ASXL1 | CCND2 | CUL4A | FGF12 | HNF1A | MAP2K1 | NF1 | PIK3R1 | RNF43 | TIPARP |
| ATM | CCND3 | CXCR4 | FGF14 | HRAS | MAP2K2 | NF2 | PIM1 | ROS1 | TNFAIP3 |
| ATR | CCNE1 | CYP17A1 | FGF19 | HSD3B1 | MAP2K4 | NFE2L2 | PMS2 | RPTOR | TNFRSF14 |
| ATRX | CD22 | DAXX | FGF23 | ID3 | MAP3K1 | NFKBIA | POLD1 | SDHA | TP53 |
| AURKA | CD274 | DDR1 | FGF3 | IDH1 | MAP3K13 | NKX2-1 | POLE | SDHB | TSC1 |
| AURKB | CD70 | DDR2 | FGF4 | IDH2 | MAPK1 | NOTCH1 | PPARG | SDHC | TSC2 |
| AXIN1 | CD79A | DIS3 | FGF6 | IGF1R | MCL1 | NOTCH2 | PPP2R1A | SDHD | TYRO3 |
| AXL | CD79B | DNMT3A | FGFR1 | IKBKE | MDM2 | NOTCH3 | PPP2R2A | SETD2 | U2AF1 |
| BAP1 | CDC73 | DOT1L | FGFR2 | IKZF1 | MDM4 | NPM1 | PRDM1 | SF3B1 | VEGFA |
| BARD1 | CDH1 | EED | FGFR3 | INPP4B | MED12 | NRAS | PRKAR1A | SGK1 | VHL |
| BCL2 | CDK12 | EGFR | FGFR4 | IRF2 | MEF2B | NT5C2 | PRKCI | SMAD2 | WHSC1 |
| BCL2L1 | CDK4 | EP300 | FH | IRF4 | MEN1 | NTRK1 | PTCH1 | SMAD4 | WHSC1L1 |
| BCL2L2 | CDK6 | EPHA3 | FLCN | IRS2 | MERTK | NTRK2 | PTEN | SMARCA4 | WT1 |
| BCL6 | CDK8 | EPHB1 | FLT1 | JAK1 | MET | NTRK3 | PTPN11 | SMARCB1 | XPO1 |
| BCOR | CDKN1A | EPHB4 | FLT3 | JAK2 | MITF | P2RY8 | PTPRO | SMO | XRCC2 |
| BCORL1 | CDKN1B | ERBB2 | FOXL2 | JAK3 | MKNK1 | PALB2 | QKI | SNCAIP | ZNF217 |
| BRAF | CDKN2A | ERBB3 | FUBP1 | JUN | MLH1 | PARK2 | RAC1 | SOCS1 | ZNF703 |
| BRCA1 | CDKN2B | ERBB4 | GABRA6 | KDM5A | MPL | PARP1 | RAD21 | SOX2 | |

TABLE 13
Genes with selected intronic regions for the detection of gene rearrangements,
one with 3′UTR, one gene with a promoter region and one ncRNA gene.

| ALK | BRCA1 | ETV4 | EZR | KIT | MYC | NUTM1 | RET | SLC34A2 |
|---|---|---|---|---|---|---|---|---|
| introns | introns | introns | introns | intron 16 | intron 1 | intron 1 | introns | intron 4 |
| 18, 19 | 2, 7, 8, 12, | 5,6 | 9-11 | | | | 7-11 | |
| | 16, 19, 20 | | | | | | | |
| BCL2 | BRCA2 | ETV5 | FGFR1 | KMT2A | | PDGFRA | ROS1 | TERC |
| 3′UTR | intron 2 | introns | intron | (MLL) | NOTCH2 | introns | introns | ncRNA |
| | | 6, 7 | 1, 5, 17 | introns | intron 26 | 7, 9, 11 | 31-35 | |
| | | | | 6-11 | | | | |
| BCR | CD74 | ETV6 | FGFR2 | MSH2 | NTRK1 | RAF1 | RSPO2 | TERT |
| introns | introns | introns | intron | intron 5 | introns | introns | intron 1 | Promoter |
| 8, 13, 14 | 6-8 | 5, 6 | 1, 17 | | 8-10 | 4-8 | | |
| BRAF | EGFR | EWSR1 | FGFR3 | MYB | NTRK2 | RARA | SDC4 | TMPRSS2 |
| introns | introns | introns | intron 17 | intron 14 | Intron 12 | intron 2 | intron 2 | introns |
| 7-10 | 7, 15, 24-27 | 7-13 | | | | | | 1-3 |

The F1CDx assay identifies various alterations in the gene and/or intron sequences, including substitutions, insertions/deletions, and CNAs. The F1CDx assay was previously identifies as having concordance with an externally validated NGS assay and the FOUNDATIONONE® (F1 LDT) assay. See FOUNDATIONONE® CDX™: Technical Information, Foundation Medicine, Inc., available at FoundationMedicine.com, last visited Mar. 25, 2019, which is incorporated by reference herein in its entirety.

### MSK-IMPACT™

In some embodiments, TMB status is assessed using the MSK-IMPACT™ assay. The MSK-IMPACT™ assay uses next-generation sequencing to analyze the mutation status of 468 genes. Target genes are captured and sequenced on an ILLUMINA HISEQ™ instrument. The MSK-IMPACT™ assay is approved by the US FDA for detection of somatic mutations and microsatellite instability in solid malignant neoplasms. The full list of 468 genes analyzed by the MSK-IMPACT™ assay is shown in Table 14. See Evaluation of Automatic Class III Designation for MSK-IMPACT (Integrated Mutation Profiling of Actionable Cancer Targets): Decision Summary, United States Food and Drug Administration, Nov. 15, 2017, available at accessdata.fda.gov.

TABLE 14
Genes analyzed by the MSK-IMPACT ™ assay.

| | | | | | | | | | |
|---|---|---|---|---|---|---|---|---|---|
| ABL1 | CALR | DDR2 | FGF19 | HIST3H3 | LYN | NKX2-1 | PPARG | RPTOR | STK19 |
| ACVR1 | CARD11 | DICER1 | FGF3 | HLA-A | MALT1 | NKX3-1 | PPM1D | RRAGC | STK40 |
| AGO2 | CARM1 | DIS3 | FGF4 | HLA-B | MAP2K1 | NOTCH1 | PPP2 | RRAS | SUFU |
| | | | | | | | R1A | | |
| AKT1 | CASP8 | DNAJB1 | FGFR1 | HNF1A | MAP2K2 | NOTCH2 | PPP4R2 | RRAS2 | SUZ12 |
| AKT2 | CBFB | DNMT1 | FGFR2 | HOXB13 | MAP2K4 | NOTCH3 | PPP6C | RTEL1 | SYK |
| AKT3 | CBL | DNMT3A | FGFR3 | HRAS | MAP3K1 | NOTCH4 | PRDM1 | RUNX1 | TAP1 |
| ALK | CCND1 | DNMT3B | FGFR4 | ICOSLG | MAP3K13 | NPM1 | PRDM14 | RXRA | TAP2 |
| ALOX | CCND2 | DOT1L | FH | ID3 | MAP3 | NRAS | PREX2 | RYBP | TBX3 |
| 12B | | | | | K14 | | | | |
| AMER1 | CCND3 | DROSHA | FLCN | IDH1 | MAPK1 | NSD1 | PRKAR1A | SDHA | TCEB1 |
| ANKRD11 | CCNE1 | DUSP4 | FLT1 | IDH2 | MAPK3 | NTHL1 | PRKCI | SDHAF2 | TCF3 |
| APC | CD274 | E2F3 | FLT3 | IFNGR1 | MAPKA | NTRK1 | PRKD1 | SDHB | TCF7L2 |
| | | | | | P1 | | | | |
| AR | CD276 | EED | FLT4 | IGF1 | MAX | NTRK2 | PTCH1 | SDHC | TEK |
| ARAF | CD79A | EGFL7 | FOXA1 | IGF1R | MCL1 | NTRK3 | PTEN | SDHD | TERT |
| ARIDIA | CD79B | EGFR | FOXL2 | IGF2 | MDC1 | NUF2 | PTP4A1 | SESN1 | TET1 |
| ARID1B | CDC42 | EIFlAX | FOXO1 | IKBKE | MDM2 | NUP93 | PTPN11 | SESN2 | TET2 |
| ARID2 | CDC73 | EIF4A2 | FOXPI | IKZFI | MDM4 | PAK1 | PTPRD | SESN3 | TGFBR1 |
| ARID5B | CDH1 | ElF4E | FUBP1 | IL10 | MED12 | PAK7 | PTPRS | SETD2 | TGFBR2 |
| ASXL1 | CDK12 | ELF3 | FYN | IL7R | MEF2B | PALB2 | PTPRT | SETD8 | TMEM127 |
| ASXL2 | CDK4 | EP300 | GATA1 | INHA | MEN1 | PARK2 | RAB35 | SF3B1 | TMPRSS2 |
| ATM | CDK6 | EPAS1 | GATA2 | INHBA | MET | PARP1 | RAC1 | SH2B3 | TNFAIP3 |
| ATR | CDK8 | EPCAM | GATA3 | INPP4A | MGA | PAX5 | RAC2 | SH2D1A | TNFRSF |
| | | | | | | | | | 14 |
| ATRX | CDKNlA | EPHA3 | GLI1 | INPP4B | MITF | PBRM1 | RAD21 | SHOC2 | TOP1 |
| AURKA | CDKN1B | EPHA5 | GNA11 | INPPL1 | MLH1 | PDCD1 | RAD50 | SHQ1 | TP53 |
| AURKB | CDKN2A | EPHA7 | GNAQ | INSR | MPL | PDCD1L | RAD51 | SLX4 | TP53BP1 |
| | p14ARF | | | | | G2 | | | |
| AXIN1 | CDKN2A | EPHB1 | GNAS | IRF4 | MRE11A | PDGFRA | RAD51B | SMAD2 | TP63 |
| | p16INK4A | | | | | | | | |
| AXIN2 | CDKN2B | ERBB2 | GPS2 | IRS1 | MSH2 | PDGFRB | RAD51C | SMAD3 | TRAF2 |
| AXL | CDKN2C | ERBB3 | GREM1 | IRS2 | MSH3 | PDPK1 | RAD51D | SMAD4 | TRAF7 |
| B2M | CEBPA | ERBB4 | GRIN2A | JAK1 | MSH6 | PGR | RAD52 | SMARCA4 | TSC1 |
| BABAM1 | CENPA | ERCC2 | GSK3B | JAK2 | MSI1 | PHOX2B | RAD54L | SMARCB1 | TSC2 |
| BAP1 | CHEK1 | ERCC3 | H3F3A | JAK3 | MSI2 | PIK3C2G | RAF1 | SMARCD1 | TSHR |
| BARD1 | CHEK2 | ERCC4 | H3F3B | JUN | MST1 | PIK3C3 | RARA | SMO | U2AF1 |
| BBC3 | CIC | ERCC5 | H3F3C | KDM5A | MST1R | PIK3CA | RASA1 | SMYD3 | UPF1 |
| BCL10 | CREBBP | ERF | HGF | KDM5C | MTOR | PIK3CB | RB1 | SOCS1 | VEGFA |
| BCL2 | CRKL | ERG | HIST1H1C | KDM6A | MUTYH | PIK3CD | RBM10 | SOS1 | VHL |
| BCL2L1 | CRLF2 | ERRFI1 | HIST1H2BD | KDR | MYC | PIK3CG | RECQL | SOX17 | VTCN1 |
| BCL2L11 | CSDE1 | ESR1 | HIST1H3A | KEAP1 | MYCL1 | PIK3R1 | RECQL4 | SOX2 | WHSC1 |
| BCL6 | CSF1R | ETV1 | HIST1H3B | KIT | MYCN | PIK3R2 | REL | SOX9 | WHSC1L1 |
| BCOR | CSF3R | ETV6 | HIST1H3C | KLF4 | MYD88 | PIK3R3 | RET | SPEN | WT1 |
| BIRC3 | CTCF | EZH1 | HIST1H3D | KMT2A | MYOD1 | PIM1 | RFWD2 | SPOP | WWTR1 |
| BLM | CLTA-4 | EZH2 | HIST1H3E | KMT2B | NBN | PLCG2 | RHEB | SPRED1 | XIAP |
| BMPR1A | CTNNB1 | FAM175A | HIST1H3F | KMT2C | NCOA3 | PLK2 | RHOA | SRC | XPO1 |
| BRAF | CUL3 | FAM46C | HIST1H3G | KMT2D | NCOR1 | PMAIP1 | RICTOR | SRSF2 | XRCC2 |
| BRCA1 | CXCR4 | FAM58A | HIST1H3H | KNSTRN | NEGR1 | PMS1 | RIT1 | STAG2 | YAP1 |
| BRCA2 | CYLD | FANCA | HIST1H3I | KRAS | NF1 | PMS2 | RNF43 | STAT3 | YES1 |
| BRD4 | CYSLTR2 | FANCC | HIST1H3J | LATS1 | NF2 | PNRC1 | ROS1 | STAT5A | ZFHX3 |
| BRIP1 | DAXX | FAT1 | HIST2H3C | LATS2 | NFE2L2 | POLD1 | RPS6KA4 | STAT5B | |
| BTK | DCUN1D1 | FBXW7 | HIST2H3D | LMO1 | NFKBIA | POLE | RPS6KB2 | STK11 | |
| ABL1 | CALR | DDR2 | FGF19 | HIST3H3 | LYN | NKX2-1 | PPARG | RPTOR | STK19 |

### NEOGENOMICS® NEOTYPE™ Assays

In some embodiments, TMB is determined using a NEOGENOMICS® NEOTYPE™ assay. In some embodiments, the TMB is determined using a NEOTYPE™ Discovery Profile. In some embodiments, the TMB is determined using a NEOTYPE Solid Tumor Profile. The NEOGENOMICS assays measure the number of non-synonymous DNA coding sequence changes per megabase of sequenced DNA.

### ONCOMINE™ Tumor Mutation Load Assay

In some embodiments, TMB is determined using a THERMOFISHER SCIENTIFIC® ONCOMINE™ Tumor Mutation assay. In some embodiments, TMB is determined using a THERMOFISHER SCIENTIFIC® ION TORRENT™ ONCOMINE™ Tumor Mutation assay. The ION TORRENT™ ONCOMINE™ Tumor Mutation assay is a targeted NGS assay that quantitates somatic mutations to determine tumor mutation load. The assay covers 1.7 Mb of DNA. The full list of 408 genes analyzed by the THERMOFISHER SCIENTIFIC® ION TORRENT™ ONCOMINE™ Tumor Mutation assay is shown in Table 15 (see Iontorrent, Oncomine Tumor Mutation Load Assay Flyer, available at assets.thermofisher.com/TFS-Assets/CSD/Flyers/oncomine-tumor-mutation-load-assay-flyer.pdf, last visited Mar. 25, 2019).

TABLE 15
Genes analyzed by the THERMOFISHER SCIENTIFIC ® ION
TORRENT ™ ONCOMINE ™ Tumor Mutation assay.

| | | | | | | | | | |
|---|---|---|---|---|---|---|---|---|---|
| 0082 | ATR | CSF1R | FAM123B | HRAS | LRP18 | MY8 | PDGFRB | RHOH | TCF7L1 |
| SEPT9 | ATRX | CSMD3 | FANCA | HSP90A81 | LTF | MYCL1 | PER1 | RNASEL | TCF7L2 |
| 81RC2 | AURK8 | CTNNA1 | FANCC | HSP90AA1 | LTK | MYCN | PGAP3 | RNF2 | TCL1A |
| 81RC3 | AURKA | CTNNB1 | FANCD2 | ICK | M8D1 | MYD88 | PHOX28 | RNF213 | TET1 |
| 81RC5 | AURKC | CYLD | FANCF | IDH1 | MAF | MYH11 | PIK3C28 | ROS1 | TET2 |
| 8AI3 | AXL | CYP2C19 | FANCG | IDH2 | MAF8 | MYH9 | PIK3CA | RPS6KA2 | TFE3 |
| 8CL10 | BAP1 | CYP2D6 | FANCJ | IGF1R | MAGEA1 | NBN | PIK3CB | RRM1 | TGF8R2 |
| 8CL118 | BRAF | DAXX | FAS | IGF2 | MAGl1 | NCOA1 | PIK3CD | RUNX1 | TGM7 |
| 8CL11A | CARD11 | DCC | FBXW7 | IGF2R | MALT1 | NCOA2 | PIK3CG | RUNX1T1 | TH8S1 |
| 8CL2 | CASCS | DDIT3 | FGFR1 | IKBKB | MAML2 | NCOA4 | PIK3R1 | SAMD9 | TIMP3 |
| 8CL2L1 | CBL | DDR2 | FGFR2 | IKBKE | MAP2K1 | NF1 | PIK3R2 | SBDS | TLR4 |
| 8CL2L2 | CCND1 | DEK | FGFR3 | IKZF1 | MAP2K2 | NF2 | PIM1 | SDHA | TLX1 |
| 8CL3 | CCND2 | DICER1 | FGFR4 | IL2 | MAP2K4 | NFE2L2 | PKHD1 | SDHB | TNFAIP3 |
| 8CL6 | CCNE1 | DNMT3A | FH | IL21R | MAP3K7 | NFK81 | PLAG1 | SDHD | TNFRSF14 |
| 8CL9 | CD79A | DPYD | FLCN | IL6ST | MAPK1 | NFK82 | PLCG1 | SETD2 | TNK2 |
| 8CR | CD79B | DST | FLl1 | IL7R | MAPK8 | NIN | PLEKHGS | SF3B1 | TOP1 |
| 8LM | CDC73 | EGFR | FLT1 | ING4 | MARK1 | NKX2-1 | PML | SGK1 | TP53 |
| 8LNK | CDH1 | EML4 | FLT3 | IRF4 | MARK4 | NLRP1 | PMS1 | SH2D1A | TPR |
| 8MPR1A | CDH11 | EP300 | FLT4 | IRS2 | MCL1 | NOTCH1 | PMS2 | SMAD2 | TRIM24 |
| 8RD3 | CDH2 | EP400 | FN1 | ITGA10 | MDM2 | NOTCH2 | POT1 | SMAD4 | TRIM33 |
| 8TK | CDH20 | EPHA3 | FOX01 | ITGA9 | MDM4 | NOTCH4 | POU5F1 | SMARCA4 | TRIP11 |
| 8U818 | CDH5 | EPHA7 | FOX03 | ITGB2 | MEN1 | NPM1 | PPARG | SMARCB1 | TRRAP |
| A8L2 | CDK12 | EPHB1 | FOXL2 | ITGB3 | MET | NRAS | PPP2R1A | SMO | TSC1 |
| ABL1 | CDK4 | EPHB4 | FOXP1 | JAK1 | MITF | NSD1 | PRDM1 | SMUG1 | TSC2 |
| ACVR24 | CDK6 | EPHB6 | FOXP4 | JAK2 | MLH1 | NTRK1 | PRKAR1A | SOCS1 | TSHR |
| ADAMTS2 | CDK8 | ERBB2 | FZR1 | JAK3 | MLL | NTRK3 | PRKDC | SOHO | U8R5 |
| AFF1 | CDKN2A | ERBB3 | G6PD | JUN | MLL2 | NUMA1 | PSIP1 | SOX11 | UGT1A1 |
| AFF3 | CDKN2B | ERBB4 | GATA1 | KAT6A | MLL3 | NUP214 | PTCH1 | SOX2 | USP9X |
| AKAP9 | CDKN2C | ERCC1 | GATA2 | KAT6B | MLLT10 | NUP98 | PTEN | SRC | VHL |
| AKT1 | CEBPA | ERCC2 | GATA3 | KDM5C | MMP2 | P8RM1 | PTGS2 | SSX1 | WAS |
| AKT2 | CHEK1 | ERCC3 | GDNF | KDM6A | MN1 | P8X1 | PTPN11 | STK11 | WHSC1 |
| AKT3 | CHEK2 | ERCC4 | GNA11 | KEAP1 | MPL | PAK3 | PTPRD | STK36 | WRN |
| ALK | CIC | ERCC5 | GNAQ | KIT | MRE11A | PALB2 | PTPRT | SUFU | WT1 |
| APC | CKS1B | ERG | GNAS | KLF6 | MSH2 | PARP1 | RADSO | SYK | XP01 |
| AR | CMPK1 | ESR1 | GPR124 | KOR | MSH6 | PAX? | RAF1 | SYNE1 | XPA |
| ARID1A | COL1A1 | ETS1 | GRM8 | KRAS | MTC | PAX3 | RALGDS | T8X22 | XPC |
| ARID2 | CRBN | ETV1 | HCAR1 | LAMP1 | MTOT | PAX8 | RARA | TAF1 | XRCC2 |
| ARNT | CREB1 | ETV4 | HFN1A | LCK | MTR | PAXS | RB1 | TAF1L | ZNF384 |
| ASXL1 | CREBBP | EXT1 | HIF1A | LIFR | MTRR | PDE4DIP | RECQL4 | TAL1 | ZNF521 |
| ATF1 | CRKL | EXT2 | HLF | LPHN3 | MUC1 | PDGF8 | REL | TCF12 | |
| ATM | CRTC1 | EZH2 | HOOK3 | LPP | MUTYH | PDGFRA | RET | TCF3 | |

### NOVOGENE™ NOVOPM™ Assay

In some embodiments, TMB is determined using a NOVOGENE™ NOVOPM™ assay. In some embodiments, TMB is determined using a NOVOGENE™ NOVOPM™ Cancer Panel assay. The NOVOGENE™ NOVOPM™ Cancer Panel assay is a comprehensive NGS cancer panel that analyzes the complete coding regions of 548 genes and the introns of 21 genes, representing about 1.5 Mb of DNA, and that are relevant for the diagnosis and/or treatment of solid tumors according to the National Comprehensive Cancer Network (NCCN) guidelines and medical literature. The assay detects SNV, InDel, fusion, and copy number variation (CNV) genomic abnormalities.

### Other TMB Assays

In some embodiments, TMB is determined using a TMB assay provided by CARIS® Life Sciences. In some embodiments, TMB is determined using the PESONALIS® ACE ImmunolD assay. In some embodiments, TMB is determined using the PGDX® CANCERXOME™-R assay.

In yet another particular embodiment, the genomic profiling detects all mutation types, i.e., single nucleotide variants, insertions/deletions (indels), copy number variations, and rearrangements, e.g., translocations, expression, and epigenetic markers.

Comprehensive gene panels often contain predetermined genes selected based on the type of tumor to be analyzed. Accordingly, the genomic profile used to measure TMB status can be selected based on the type of tumor the subject has. In one embodiment, the genomic profile can include a set of genes particular to a solid tumor. In another embodiment, the genomic profile can include a set of genes particular to hematologic malignancies and sarcomas.

In one embodiment, the genomic profile comprises one or more genes selected from the group consisting of ABL1, BRAF, CHEK1, FANCC, GATA3, JAK2, MITF, PDCD1LG2, RBM10, STAT4, ABL2, BRCA1, CHEK2, FANCD2, GATA4, JAK3, MLH1, PDGFRA, RET, STK11, ACVR1B, BRCA2, CIC, FANCE, GATA6, JUN, MPL, PDGFRB, RICTOR, SUFU, AKT1, BRD4, CREBBP, FANCF, GID4 (C17orf39), KAT6A (MYST3), MRE11A, PDK1, RNF43, SYK, AKT2, BRIP1, CRKL, FANCG, GLI1, KDM5A, MSH2, PIK3C2B, ROS1, TAF1, AKT3, BTG1, CRLF2, FANCL, GNA11, KDM5C, MSH6, PIK3CA, RPTOR, TBX3, ALK, BTK, CSF1R, FAS, GNA13, KDM6A, MTOR, PIK3CB, RUNX1, TERC, AMER1 (FAM123B), C11orf30 (EMSY), CTCF, FAT1, GNAQ, KDR, MUTYH, PIK3CG, RUNX1T1, TERT (promoter only), APC, CARD11, CTNNA1, FBXW7, GNAS, KEAP1, MYC, PIK3R1, SDHA, TET2, AR, CBFB, CTNN B1, FGF10, GPR124, KEL, MYCL (MYCL1), PIK3R2, SDHB, TGFBR2, ARAF, CBL, CUL3, FGF14, GRIN2A, KIT, MYCN, PLCG2, SDHC, TNFAIP3, ARFRP1, CCND1, CYLD, FGF19, GRM3, KLHL6, MYD88, PMS2, SDHD, TNFRSF14, ARID1A, CCND2, DAXX, FGF23, GSK3B, KMT2A (MLL), NF1, POLD1, SETD2, TOP1, ARID1B, CCND3, DDR2, FGF3, H3F3A, KMT2C (MLL3), NF2, POLE, SF3B1, TOP2A, ARID2, CCNE1, DICER1, FGF4, HGF, KMT2D (MLL2), NFE2L2, PPP2R1A, SLIT2, TP53, ASXL1, CD274, DNMT3A, FGF6, HNF1A, KRAS, NFKB1A, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX, CDC73, EP300, FGFR3, HSP90AA1, LYN, NOTCH2, PRKC1, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLCN, IGF1R, MAP2K1, NRAS, PTCH1, SNCAIP, WISP3, AXL, CDK6, EPHB1, FLT1, IGF2, MAP2K2, NSD1, PTEN, SOCS1, WT1, BAP1, CDK8, ERBB2, FLT3, IKBKE, MAP2K4, NTRK1, PTPN11, SOX10, XPO1, BARD1, CDKN1A, ERBB3, FLT4, IKZF1, MAP3K1, NTRK2, QKI, SOX2, ZBTB2, BCL2, CDKN1B, ERBB4, FOXL2, IL7R, MCL1, NTRK3, RAC1, SOX9, ZNF217, BCL2L1, CDKNV2A, ERG, FOXP1, INHBA, MDM2, NUP93, RAD50, SPEN, ZNF703, BCL2L2, CDKN2B, ERRFI1, FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRMN, RB1, STAT3, and any combination thereof. In other embodiments, the TMB analysis further comprises identifying a genomic alteration in one or more of ETV4, TMPRSS2, ETV5, BCR, ETV1, ETV6, and MYB.

In another embodiment, the genomic profile comprises one or more genes selected from the group consisting of ABL1, 12B, ABL2, ACTB, ACVR1, ACVR1B, AGO2, AKT1, AKT2, AKT3, ALK, ALOX; ALOX12B, AMER1, AMER1 (FAM123B or WTX), AMER1 (FAM123B), ANKRD11, APC, APH1A, AR, ARAF, ARFRP1, ARHGAP26 (GRAF), ARID1A, ARID1B, ARID2, ARID5B, ARv7, ASMTL, ASXL1, ASXL2, ATM, ATR, ATRX, AURKA, AURKB, AXIN1, AXIN2, AXL, B2M, BABAM1, BAP1, BARD1, BBC3, BCL10, BCL11B, BCL2, BCL2L1, BCL2L11, BCL2L2, BCL6, BCL7A, BCOR, BCORL1, BIRC3, BLM, BMPR1A, BRAF, BRCA1, BRCA2, BRD4, BRIP1, BRIP1 (BACH1), BRSK1, BTG1, BTG2, BTK, BTLA, C11orf 30 (EMSY), C11orf30, C11orf30 (EMSY), CAD, CALR, CARD1, CARMI, CASP8, CBFB, CBL, CCND1, CCND2, CCND3, CCNE1, CCT6B, CD22, CD274, CD274 (PD-L1), CD276, CD36, CD58, CD70, CD79A, CD79B, CDC42, CDC73, CDH1, CDK12, CDK4, CDK6, CDK8, CDKN1A, CDKN1B, CDKN2A, CDKN2Ap14ARF, CDKN2Ap16INK4A, CDKN2B, CDKN2C, CEBPA, CENPA, CHD2, CHD4, CHEK1, CHEK2, CIC, CIITA, CKS1B, CPS1, CREBBP, CRKL, CRLF2, CSDE, CSF1R, CSF3R, CTCF, CLTA-4, CTNN B1, CTNNA1, CTNNB, CUL3, CUL4A, CUX1, CXCR4, CYLD, CYP17A1, CYSLTR2, DAXX, DCUN1D1, DDR1, DDR2, DDX3X, DH2, DICER1, DIS3, DNAJB1, DNM2, DNMT1, DNMT3A, DNMT3B, DOT1L, DROSHA, DTX1, DUSP2, DUSP4, DUSP9, E2F3, EBF1, ECT2L, EED, EGFL7, EGFR, EIF1AX, EIF4A2, EIF4E, ELF3, ELP2, EML4, EML4-ALK, EP300, EPAS, EPCAM, EPHA3, EPHA5, EPHA7, EPHB, EPHB4, ERBB2, ERBB3, ERBB4, ERCC, ERCC2, ERCC3, ERCC4, ERCC5, ERF, ERG, ERRFI1, ERRFI1, ESR1, ETS1, ETV1, ETV4, ETV5, ETV6, EWSR1, EXOSC6, EZH, EZH2, FAF, FAM175A, FAM46C, FAM58A, FANCA, FANCC, FANCD2, FANCE, FANCF, FANCG, FANCI, FANCL, FAS, FAS (TNFRSF6), FAT1, FBXO11, FBXO31, FBXW7, FGF1, FGF10, FGF12, FGF14, FGF19, FGF2, FGF23, FGF3, FGF4, FGF5, FGF6, FGF7, FGF8, FGF9, FGFR1, FGFR2, FGFR3, FGFR4, FH, FHIT, FLCN, FLI1, FLT1, FLT3, FLT4, FLYWCH1, FOXA1, FOXL2, FOXO1, FOXO3, FOXP1, FRS2, FUBP1, FYN, GABRA6, GADD45B, GATA1, GATA2, GATA3, GATA4, GATA6, GEN1, GID4 (C17orf 39), GID4 (C17orf39), GLI1, GLl1, GNA11, GNA12, GNA13, GNAQ, GNAS, GPR124, GPS2, GREM1, GRIN2A, GRM3, GSK3B, GTSE1, H3F3A, H3F3B, H3F3C, HDAC1, HDAC4, HDAC7, Hedgehog, HER-2/NEU; ERBB2, HGF, HIST1H1C, HIST1H1D, HIST1H1E, HIST1H2AC, HIST1H2AG, HIST1H2AL, HIST1H2AM, HIST1H2BC, HIST1H2BD, HIST1H2BJ, HIST1H2BK, HIST1H2BO, HIST1H3A, HIST1H3B, HIST1H3C, HIST1H3D, HIST1H3E, HIST1H3F, HIST1H3G, HIST1H3H, HIST1H3I, HIST1H3J, HIST2H3C, HIST2H3D, HIST3H3, HLA-A, HLA-B, HNF1A, HOXB13, HRAS, HSD3B1, HSP90AA1, ICK, ICOSLG, ID3, IDH1, IDH2, IFNGR1, IGF1, IGF1R, IGF2, IKBKE, IKZF1, IKZF2, IKZF3, IL10, IL7R, INHA, INHBA, INPP4A, INPP4B, INPP5D (SHIP), INPPL1, INSR, IRF1, IRF2, IRF4, IRF8, IRS1, IRS2, JAK1, JAK2, JAK3, JARID2, JUN, K14, KAT6A (MYST 3), KAT6A (MYST3), KDM2B, KDM4C, KDM5A, KDM5C, KDM6A, KDR, KEAP1, KEL, KIF5B, KIT, KLF4, KLHL6, KMT2A, KMT2A (MLL), KMT2B, KMT2C, KMT2C (MLL3), KMT2D, KMT2D (MLL2), KNSTRN, KRAS, LAMP1, LATS1, LATS2, LEF1, LMO1, LRP1B, LRRK2, LTK, LYN, LZTR1, MAF, MAFB, MAGED1, MAGI2, MALT1, MAP2K1, MAP2K (MEK1), MAP2K2, MAP2K2 (MEK2), MAP2K4, MAP3, MAP3K1, MAP3K13, MAP3K14, MAP3K6, MAP3K7, MAPK1, MAPK3, MAPKAP1, MAX MCL1, MDC1, MDM2, MDM4, MED12, MEF2B, MEF2C, MEK1, MEN1, MERTK, MET, MGA, MIB1, MITF, MKI67, MKNK1, MLH1, MLLT3, MPL, MRE 11A, MRE11A, MSH2, MSH3, MSH6, MSI1, MSI2, MST1, MST1R, MTAP, MTOR, MUTYH, MYC, MYCL, MYCL (MYC L1), MYCL (MYCL1), MYCL1, MYCN, MYD88, MYO18A, MYOD1, NBN, NCOA3, NCOR1, NCOR2, NCSTN, NEGR1, NF1, NF2, NFE2L2, NFKB1A, NKX2-1, NKX3-1, NOD1, NOTCH1, NOTCH2, NOTCH3, NOTCH4, NPM1, NRAS, NRG1, NSD1, NT5C2, NTHL1, NTRK1, NTRK2, NTRK3, NUF2, NUP93, NUP98, P2RY8, PAG1, PAK1, PAK3, PAK7, PALB2, PARK2, PARP1, PARP2, PARP3, PASK, PAX3, PAX5, PAX7, PBRM1, PC, PCBP1, PCLO, PDCD1, PDCD1 (PD-1), PDCD11, PDCD1LG2, PDCD1LG2 (PD-L2), PDGFRA, PDGFRB, PDK1, PDPK1, PGR, PHF6, PHOX2B, PIK3C2B, PIK3C2G, PIK3C3, PIK3CA, PIK3CB, PIK3CD, PIK3CG, PIK3R1, PIK3R2, PIK3R3, PIM1, PLCG2, PLK2, PMAIP1, PMS1, PMS2, PNRC1, POLD1, POLE, POT1, PPARG, PPM1D, PPP2, PPP2R1A, PPP2R2A, PPP4R2, PPP6C, PRDM1, PRDM14, PREX2, PRKAR1A, PRKC1, PRKD1, PRKDC, PRSS8, PTCH1, PTEN, PTP4A1, PTPN11, PTPN2, PTPN6 (SHP-1), PTPRD, PTPRO, PTPRS, PTPRT, QKI, R1A, RAB35, RAC1, RAC2, RAD21, RAD50, RAD51, RAD51B, RAD51C, RAD51D, RAD52, RAD54L, RAF1, RANBP2, RARA, RASA1, RASGEF1A, RB1, RBM10, RECQL, RECQL4, REL, RELN, RET, RFWD2, RHEB, RHOA, RICTOR, RIT1, RNF43, ROS1, RPS6KA4, RPS6 KB1, RPS6KB2, RPTOR, RRAGC, RRAS, RRAS2, RTEL1, RUNX1, RUNX1T1, RXRA, RYBP, S1PR2, SDHA, SDHAF2, SDHB, SDHC, SDHD, SERP2, SESN1, SESN2, SESN3, SETBP1, SETD2, SETD8, SF3B1, SGK1, SH2B3, SH2D1A, SHOC2, SHQ1, SLIT2, SLX4, SMAD2, SMAD3, SMAD4, SMARCA, SMARCA4, SMARCB1, SMARCD1, SMC1A, SMC3, SMO, SMYD3, SNCAIP, SOCS1, SOCS2, SOCS3, SOS1, SOX10, SOX17, SOX2, SOX9, SPEN, SPOP, SPRED1, SPTA1, SRC, SRSF2, STAG2, STAT3, STAT4, STAT5A, STAT5B, STAT6, STK11, STK19, STK40, SUFU, SUZ12, SYK, TAF1, TAP1, TAP2, TBL1XR1, TBX3, TCEB1, TCF3, TCF3 (E2A), TCF7L2, TCL1A (TCL1), TEK, TERC, TERT, TERT Promoter, TET1, TET2, TFRC, TGFBR1, TGFBR2, TIPARP, TLL2, TMEM127, TMEM30A, TMPRSS2, TMSB4XP8 (TMSL3), TNFAIP3, TNFRSF1A, TNFRSF4, TNFRSF7, TOP1, TOP2A, TP53, TP53BP1, TP63, TRAF2, TRAF3, TRAF5, TRAF7, TSC1, TSC2, TSHR, TUSC3, TYK2, TYRO3, U2AF1, U2AF2, UPF1, VEGFA, VHL, VTCN1, WDR90, WHSC1, WHSC1 (MMSET or NSD2), WHSC1L1, WISP3, WT1, WWTR1, XBP1, XIAP, XPO1, XRCC2, YAP1, YES1, YY1AP1, ZBTB2, ZFHX3, ZMYM3, ZNF217, ZNF24 (ZSCAN3), ZNF703, ZRSR2, 0082, SEPT9, 81RC2, 81RC3, 81RC5, 8AI3, 8CL10, 8CL118, 8CL11A, 8CL2, 8CL2L1, 8CL2L2, 8CL3, 8CL6, 8CL9, 8CR, 8LM, 8LNK, 8MPR1A, 8RD3, 8TK, 8U818, A8L2, ACVR2A, ADAMTS2, AFF1, AFF3, AKAP9, ARNT, ATF1, AURK8, AURKC, CASCS, CDH11, CDH2, CDH20, CDH5, CMPK1, COL1A1, CRBN, CREB1, CRTC1, CSMD3, CYP2C19, CYP2D6, DCC, DDIT3, DEK, DPYD, DST, EP400, EXT1, EXT2, FAM123B, FANC1, FLl1, FN1, FOX01, FOX03, FOXP4, FZR1, G6PD, GDNF, GRM8, HCAR1, HFN1A, HIF1A, HLF, HOOK3, HSP90A81, ICK, IGF2R, IKBKB, IL2, IL21R, IL6ST, ING4, ITGA10, ITGA9, ITGB2, ITGB3, KAT6A, KAT6B, KLF6, KOR, LCK, LIFR, LPHN3, LPP, LRP18, LTF, M8D1, MAF8, MAGEA1, MAGI1, MAML2, MAPK8, MARK1, MARK4, MLL, MLL2, MLL3, MLLT10, MAMP2, MN1, MTC, MTOT, MTR, MTRR, MUC1, MY8, MYH11, MYH9, NCOA1, NCOA2, NCOA4, NFK81, NFK82, NIN, NLRP1, NUMA1, NUP214, P8RM1, P8X1, PAX?, PAX3, PAX8, PAXS, PDE4DIP, PDGF8, PER1, PGAP3, PHOX28, PIK3C28, PKHD1, PLAG1, PLCG1, PLEKHGS, PML, POU5F1, PSIP1, PTGS2, RADSO, RALGDS, RHOH, RNASEL, RNF2, RNF213, RPS6KA2, RRM1, SAMD9, SBDS, SMUG1, SOHO, SOX11, SSX1, STK36, SYNE1, T8X22, TAF1L, TAL1, TCF12, TCF7L1, TFE3, TGF8R2, TGM7, TH8S1, TIMP3, TLR4, TLX1, TNK2, TPR, TRIM24, TRIM33, TRIP11, TRRAP, U8R5, UGT1A1, USP9X, WAS, WRN, XP01, XPA, XPC, ZNF384, ZNF521, and any combination thereof.

In another embodiment, the genomic profiling assay comprises at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, or at least about 300 genes selected from the group consisting of ABL1, 12B, ABL2, ACTB, ACVR1, ACVR1B, AGO2, AKT1, AKT2, AKT3, ALK, ALOX ALOX12B, AMER1, AMER1 (FAM123B or WTX), AMER1 (FAM123B), ANKRD11, APC, APH1A, AR, ARAF, ARFRP1, ARHGAP26 (GRAF), ARID1A, ARID1B, ARID2, ARID5B, ARv7, ASMTL, ASXL1, ASXL2, ATM, ATR, ATRX, AURKA, AURKB, AXN1, AMN2, AXL, B2M, BABAM1, BAP1, BARD1, BBC3, BCL10, BCL11B, BCL2, BCL2L1, BCL2L11, BCL2L2, BCL6, BCL7A, BCOR, BCORL1, BIRC3, BLM, BMPR1A, BRAF, BRCA1, BRCA2, BRD4, BRIP1, BRIP1 (BACH1), BRSK1, BTG1, BTG2, BTK, BTLA, C11orf 30 (EMSY), C11orf30, C11orf30 (EMSY), CAD, CALR, CARD11, CARMI, CASP8, CBFB, CBL, CCND1, CCND2, CCND3, CCNE1, CCT6B, CD22, CD274, CD274 (PD-L1), CD276, CD36, CD58, CD70, CD79A, CD79B, CDC42, CDC73, CDH1, CDK12, CDK4, CDK6, CDK8, CDKN1A, CDKN1B, CDKN2A, CDKN2Ap14ARF, CDKNV2Ap16INK4A, CDKN2B, CDKN2C, CEBPA, CENPA, CHD2, CHD4, CHEK1, CHEK2, CIC, CIITA, CKS1B, CPS1, CREBBP, CRKL, CRLF2, CSDE1, CSF1R, CSF3R, CTCF, CLTA-4, CTNN B1, CTNNA1, CTNNB1, CUL3, CUL4A, CUX1, CXCR4, CYLD, CYP17A1, CYSLTR2, DAXX DCUN1D1, DDR1, DDR2, DDX3X, DH2, DICER1, DIS3, DNAJB1, DNM2, DNMT1, DNMT3A, DNMT3B, DOT1L, DROSHA, DTX1, DUSP2, DUSP4, DUSP9, E2F3, EBF1, ECT2L, EED, EGFL7, EGFR, EIF1AX EIF4A2, EIF4E, ELF3, ELP2, EML4, EML4-ALK, EP300, EPAS1, EPCAM, EPHA3, EPHA5, EPHA7, EPHB1, EPHB4, ERBB2, ERBB3, ERBB4, ERCC1, ERCC2, ERCC3, ERCC4, ERCC5, ERF, ERG, ERRFI1, ERRFI1, ESR1, ETS1, ETV1, ETV4, ETV5, ETV6, EWSR1, EXOSC6, EZH1, EZH2, FAF1, FAM175A, FAM46C, FAM58A, FANCA, FANCC, FANCD2, FANCE, FANCF, FANCG, FANCI, FANCL, FAS, FAS (TNFRSF6), FAT1, FBXO11, FBXO31, FBXW7, FGF1, FGF10, FGF12, FGF14, FGF19, FGF2, FGF23, FGF3, FGF4, FGF5, FGF6, FGF7, FGF8, FGF9, FGFR1, FGFR2, FGFR3, FGFR4, FH, FHIT, FLCN, FLI1, FLT1, FLT3, FLT4, FLYWCH1, FOXA1, FOXL2, FOXO1, FOXO3, FOXP1, FRS2, FUBP1, FYN, GABRA6, GADD45B, GATA1, GATA2, GATA3, GATA4, GATA6, GEN1, GID4 (C17orf 39), GID4 (C17orf39), GLI1, GLl1, GNA11, GNA12, GNA13, GNAQ, GNAS, GPR124, GPS2, GREM1, GRIN2A, GRM3, GSK3B, GTSE1, H3F3A, H3F3B, H3F3C, HDAC1, HDAC4, HDAC7, Hedgehog, HER-2/NEU; ERBB2, HGF, HIST1H1C, HIST1H1D, HIST1H1E, HIST1H2AC, HIST1H2AG, HIST1H2AL, HIST1H2AM, HIST1H2BC, HIST1H2BD, HIST1H2BJ, HIST1H2BK, HIST1H2BO, HIST1H3A, HIST1H3B, HIST1H3C, HIST1H3D, HIST1H3E, HIST1H3F, HIST1H3G, HIST1H3H, HIST1H3I, HIST1H3J, HIST2H3C, HIST2H3D, HIST3H3, HLA-A, HLA-B, HNFA, HOXB13, HRAS, HSD3B1, HSP90AA1, ICK, ICOSLG, ID3, IDH1, IDH2, IFNGR1, IGF1, IGFR, IGF2, IKBKE, IKZF, IKZF2, IKZF3, IL10, IL7R, INHA, INHBA, INPP4A, INPP4B, INPP5D (SHIP), INPPL1, INSR, IRF1, IRF2, IRF4, IRF8, IRS1, IRS2, JAK1, JAK2, JAK3, JARID2, JUN, K14, KAT6A (MYST 3), KAT6A (MYST3), KDM2B, KDM4C, KDM5A, KDM5C, KDM6A, KDR, KEAP1, KEL, KIF5B, KIT, KLF4, KLHL6, KMT2A, KMT2A (MLL), KMT2B, KMT2C, KMT2C (MLL3), KMT2D, KMT2D (MLL2), KNSTRN, KRAS, LAMP1, LATS1, LATS2, LEF1, LMO1, LRP1B, LRRK2, LTK, LYN, LZTR1, MAF, MAFB, MAGED1, MAGI2, MALT1, MAP2K1, MAP2K1 (MEK1), MAP2K2, MAP2K2 (MEK2), MAP2K4, MAP3, MAP3K1, MAP3K13, MAP3K14, MAP3K6, MAP3K7, MAPK1, MAPK3, MAPKAP1, MAX, MCL1, MDC1, MDM2, MDM4, MED12, MEF2B, MEF2C, MEK1, MEN1, MERTK, MET, MGA, MIB1, MITF, MKI67, MKNK1, MLH1, MLLT3, MPL, MRE 11A, MRE11A, MSH2, MSH3, MSH6, MSI1, MSI2, MST1, MST1R, MTAP, MTOR, MUTYH, MYC, MYCL, MYCL (MYC L1), MYCL (MYCL1), MYCL1, MYCN, MYD88, MYO18A, MYOD1, NBN, NCOA3, NCOR1, NCOR2, NCSTN, NEGR1, NF1, NF2, NFE2L2, NFKB1A, NKX2-1, NKX3-1, NOD1, NOTCH1, NOTCH2, NOTCH3, NOTCH4, NPM1, NRAS, NRG1, NSD1, NT5C2, NTHL1, NTRK1, NTRK2, NTRK3, NUF2, NUP93, NUP98, P2RY8, PAG1, PAK1, PAK3, PAK7, PALB2, PARK2, PARP1, PARP2, PARP3, PASK, PAX3, PAX5, PAX7, PBRM1, PC, PCBP1, PCLO, PDCD1, PDCD1 (PD-1), PDCD11, PDCD1LG2, PDCD1LG2 (PD-L2), PDGFRA, PDGFRB, PDK1, PDPK1, PGR, PHF6, PHOX2B, PIK3C2B, PIK3C2G, PIK3C3, PIK3CA, PIK3CB, PIK3CD, PIK3CG, PIK3R1, PIK3R2, PIK3R3, PIM1, PLCG2, PLK2, PMAIP1, PMS1, PMS2, PNRC1, POLD1, POLE, POT1, PPARG, PPM1D, PPP2, PPP2R1A, PPP2R2A, PPP4R2, PPP6C, PRDM1, PRDM14, PREX2, PRKAR1A, PRKC1, PRKD1, PRKDC, PRSS8, PTCH1, PTEN, PTP4A1, PTPN11, PTPN2, PTPN6 (SHP-1), PTPRD, PTPRO, PTPRS, PTPRT, QKI, R1A, RAB35, RAC1, RAC2, RAD21, RAD50, RAD51, RAD51B, RAD51C, RAD51D, RAD52, RAD54L, RAF1, RANBP2, RARA, RASA1, RASGEF1A, RB1, RBM10, RECQL, RECQL4, REL, RELN, RET, RFWD2, RHEB, RHOA, RICTOR, RIT1, RNF43, ROS1, RPS6KA4, RPS6 KB1, RPS6KB2, RPTOR, RRAGC, RRAS, RRAS2, RTEL1, RUNX1, RUNX1T1, RXRA, RYBP, S1PR2, SDHA, SDHAF2, SDHB, SDHC, SDHD, SERP2, SESN1, SESN2, SESN3, SETBP1, SETD2, SETD8, SF3B1, SGK1, SH2B3, SH2D1A, SHOC2, SHQ1, SLIT2, SLX4, SMAD2, SMAD3, SMAD4, SMARCA1, SMARCA4, SMARCB1, SMARCD1, SMC1A, SMC3, SMO, SMYD3, SNCAIP, SOCS1, SOCS2, SOCS3, SOS1, SOX10, SOX17, SOX2, SOX9, SPEN, SPOP, SPRED1, SPTA1, SRC, SRSF2, STAG2, STAT3, STAT4, STAT5A, STAT5B, STAT6, STK11, STK19, STK40, SUFU, SUZ12, SYK, TAF1, TAP1, TAP2, TBL1XR1, TBX3, TCEB1, TCF3, TCF3 (E2A), TCF7L2, TCL1A (TCL1), TEK, TERC, TERT, TERT Promoter, TET1, TET2, TFRC, TGFBR1, TGFBR2, TIPARP, TLL2, TMEM127, TMEM30A, TMPRSS2, TMSB4XP8 (TMSL3), TNFAIP3, TNFRSF1A, TNFRSF4, TNFRSF7, TOP1, TOP2A, TP53, TP53BP1, TP63, TRAF2, TRAF3, TRAF5, TRAF7, TSC1, TSC2, TSHR, TUSC3, TYK2, TYRO3, U2AF1, U2AF2, UPF1, VEGFA, VHL, VTCN1, WDR90, WHSC1, WHSC1 (MMSET or NSD2), WHSC1L1, WISP3, WT1, WWTR1, XBP1, MAP, XPO1, XRCC2, YAP1, YES1, YY1AP1, ZBTB2, ZFHX3, ZMYM3, ZNF217, ZNF24 (ZSCAN3), ZNF703, ZRSR2, 0082, SEPT9, 81RC2, 81RC3, 81RC5, 8AI3, 8CL10, 8CL118, 8CL11A, 8CL2, 8CL2L1, 8CL2L2, 8CL3, 8CL6, 8CL9, 8CR, 8LM, 8LNK, 8MPR1A, 8RD3, 8TK, 8U818, A8L2, ACVR2A, ADAMTS2, AFF1, AFF3, AKAP9, ARNT, ATF1, AURK8, AURKC, CASCS, CDH11, CDH2, CDH20, CDH5, CMPK1, COL1A1, CRBN, CREB1, CRTC1, CSMD3, CYP2C19, CYP2D6, DCC, DDIT3, DEK, DPYD, DST, EP400, EXT1, EXT2, FAM123B, FANC1, FLl1, FN1, FOX01, FOX03, FOXP4, FZR1, G6PD, GDNF, GRM8, HCAR1, HFN1A, HIF1A, HLF, HOOK3, HSP90A81, ICK, IGF2R, IKBKB, IL2, IL21R, IL6ST, ING4, ITGA10, ITGA9, ITGB2, ITGB3, KAT6A, KAT6B, KLF6, KOR, LCK, LIFR, LPHN3, LPP, LRP18, LTF, M8D1, MAF8, MAGEA1, MAGI1, MAML2, MAPK8, MARK1, MARK4, MLL, MLL2, MLL3, MLLT10, MMP2, MN1, MTC, MTOT, MTR, MTRR, MUC1, MY8, MYH11, MYH9, NCOA1, NCOA2, NCOA4, NFK81, NFK82, NIN, NLRP1, NUMA1, NUP214, P8RM1, P8X1, PAX?, PAX3, PAX8, PAXS, PDE4DIP, PDGF8, PER1, PGAP3, PHOX28, PIK3C28, PKHD1, PLAG1, PLCG1, PLEKHGS, PML, POU5F1, PSIP1, PTGS2, RADSO, RALGDS, RHOH, RNASEL, RNF2, RNF213, RPS6KA2, RRM1, SAMD9, SBDS, SMUG1, SOHO, SOX11, SSX1, STK36, SYNE1, T8X22, TAF1L, TAL1, TCF12, TCF7L1, TFE3, TGF8R2, TGM7, TH8S1, TIMP3, TLR4, TLX1, TNK2, TPR, TRIM24, TRIM33, TRIP11, TRRAP, U8R5, UGT1A1, USP9X, WAS, WRN, XP01, XPA, XPC, ZNF384, ZNF521, and any combination thereof.

In another embodiment, the genomic profile comprises one or more genes selected from the genes listed in Tables 2-15.

In one embodiment, TMB status based on genomic profiling is highly correlated with TMB status based on whole-exome or whole-genome sequencing. Evidence provided herein shows that the use of genomic profiling assays, such as the F1CDx assay, have concordance with whole-exome and/or whole genome sequencing assays. These data support the use of genomic profiling assays as a more efficient means of measuring TMB status, without forfeiting the prognostic qualities of TMB status.

TMB can be measured using a tissue biopsy sample or, alternatively, circulating tumor DNA (ctDNA), cfDNA (cell-free DNA), and/or a liquid biopsy sample. ctDNA can be used to measure TMB status according to whole-exome or whole-genome sequencing or genomic profiling using available methodologies, e.g., GRAIL, Inc.

A subject is identified as suitable for combination therapy comprising (a) an anti-PD-1 antibody or an anti-PD-L1 antibody and (b) an anti-CTLA-4 antibody, based on the measurement of TMB status and identification of a high TMB. In some embodiments, a TMB score is calculated as the total number of nonsynonymous missense mutations in a tumor, as measured by whole exome sequencing or whole genome sequencing. In one embodiment, the high TMB has a score of at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 445, at least 450, at least 455, at least 460, at least 465, at least 470, at least 475, at least 480, at least 485, at least 490, at least 495, or at least 500. In another embodiment, the high TMB has a score of at least 215, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, or at least 250. In a particular embodiment, the high TMB has a score of at least 243. In other embodiments, the high TMB has a score of at least 244. In some embodiments, the high TMB has a score of at least 245. In other embodiments, the high TMB has a score of at least 246. In other embodiments, the high TMB has a score of at least 247. In other embodiments, the high TMB has a score of at least 248. In other embodiments, the high TMB has a score of at least 249. In other embodiments, the high TMB has a score of at least 250. In other embodiments, the high TMB has a score of any integer between 200 and 300 or higher. In other embodiments, the high TMB has a score of any integer between 210 and 290 or higher. In other embodiments, the high TMB has a score of any integer between 220 and 280 or higher. In other embodiments, the high TMB has a score of any integer between 230 and 270 or higher. In other embodiments, the high TMB has a score of any integer between 235 and 265 or higher.

Alternatively, the high TMB can be a relative value rather than an absolute value. In some embodiments, the subject's TMB status is compared to a reference TMB value. In one embodiment, the subject's TMB status is within the highest fractile of the reference TMB value. In another embodiment, the subject's TMB status is within the top tertile of the reference TMB value.

In some embodiments, TMB status is expressed as the number of mutations per sample, per cell, per exome, or per length of DNA (e.g., Mb). In some embodiments, a tumor has a high TMB status if the tumor has at least about 50 mutations/tumor, at least about 55 mutations/tumor, at least about 60 mutations/tumor, at least about 65 mutations/tumor, at least about 70 mutations/tumor, at least about 75 mutations/tumor, at least about 80 mutations/tumor, at least about 85 mutations/tumor, at least about 90 mutations/tumor, at least about 95 mutations/tumor, at least about 100 mutations/tumor, at least about 105 mutations/tumor, at least about 110 mutations/tumor, at least about 115 mutations/tumor, or at least about 120 mutations/tumor. In some embodiments, a tumor has a high TMB status if the tumor has at least about 125 mutations/tumor, at least about 150 mutations/tumor, at least about 175 mutations/tumor, at least about 200 mutations/tumor, at least about 225 mutations/tumor, at least about 250 mutations/tumor, at least about 275 mutations/tumor, at least about 300 mutations/tumor, at least about 350 mutations/tumor, at least about 400 mutations/tumor, or at least about 500 mutations/tumor. In one particular embodiment, a tumor has a high TMB status if the tumor has at least about 100 mutations/tumor.

In some embodiments, a tumor has a high TMB status if the tumor has at least about 5 mutations per megabase of genes, e.g., genome sequenced according to a TMB assay, e.g., genome sequenced according to a FOUNDATIONONE® CDX™ assay, (mutations/Mb), at least about 6 mutations/Mb, at least about 7 mutations/Mb, at least about 8 mutations/Mb, at least about 9 mutations/Mb, at least about 10 mutations/Mb, at least about 11 mutations/Mb, at least about 12 mutations/Mb, at least about 13 mutations/Mb, at least about 14 mutations/Mb, at least about 15 mutations/Mb, at least about 20 mutations/Mb, at least about 25 mutations/Mb, at least about 30 mutations/Mb, at least about 35 mutations/Mb, at least about 40 mutations/Mb, at least about 45 mutations/Mb, at least about 50 mutations/Mb, at least about 75 mutations/Mb, or at least about 100 mutations/Mb. In certain embodiments, a tumor has a high TMB status if the tumor has at least about 5 mutations/Mb. In certain embodiments, a tumor has a high TMB status if the tumor has at least about 10 mutations/Mb. In some embodiments, a tumor has a high TMB status if the tumor has at least about 11 mutations/Mb. In some embodiments, a tumor has a high TMB status if the tumor has at least about 12 mutations/Mb. In some embodiments, a tumor has a high TMB status if the tumor has at least about 13 mutations/Mb. In some embodiments, a tumor has a high TMB status if the tumor has at least about 14 mutations/Mb. In certain embodiments, a tumor has a high TMB status if the tumor has at least about 15 mutations/Mb.

Because the number of mutations varies by tumor type and other ways (see Q4 and Q5), the values associated with “TMB high” and “TMB low” can differ across tumor types.

### PD-L1 Status

TMB status can be used alone or in combination with other factors as a means to predict a tumor's response to a combination therapy comprising (a) an anti-PD-1 antibody or an anti-PD-L1 antibody and (b) an anti-CTLA-4 antibody. In some embodiments, only the TMB status of a tumor is used to identify patients with a tumor more likely to respond to a combination therapy comprising (a) an anti-PD-1 antibody or an anti-PD-L1 antibody and (b) an anti-CTLA-4 antibody. In other embodiments, the PD-L1 status and TMB status are used to identify patients with a tumor more likely to respond to a combination therapy comprising (a) an anti-PD-1 antibody or an anti-PD-L1 antibody and (b) an anti-CTLA-4 antibody. In certain embodiments, the tumor has a PD-L1 expression of less than 1%, e.g., less than 1% of tumor cells express PD-L1. In particular embodiments, the subject has a high TMB status (≥10 mut/Mb) and a tumor PD-L1 expression level of less than 1%.

The PD-L1 status of a tumor in a subject can be measured prior to administering any composition or utilizing any method disclosed herein. PD-L1 expression can be determined by any methods known in the art.

In order to assess the PD-L1 expression, in one embodiment, a test tissue sample can be obtained from the patient who is in need of the therapy. In another embodiment, the assessment of PD-L1 expression can be achieved without obtaining a test tissue sample. In some embodiments, selecting a suitable patient includes (i) optionally providing a test tissue sample obtained from a patient having a tumor derived from a NSCLC, the test tissue sample comprising tumor cells and/or tumor-infiltrating inflammatory cells; and (ii) assessing the proportion of cells in the test tissue sample that express PD-L1 on the surface of the cells based on an assessment that the proportion of cells in the test tissue sample that express PD-L1 on the cell surface is higher than a predetermined threshold level.

In any of the methods comprising the measurement of PD-L1 expression in a test tissue sample, however, it should be understood that the step comprising the provision of a test tissue sample obtained from a patient is an optional step. It should also be understood that in certain embodiments the “measuring” or “assessing” step to identify, or determine the number or proportion of, cells in the test tissue sample that express PD-L1 on the cell surface is performed by a transformative method of assaying for PD-L1 expression, for example by performing a reverse transcriptase-polymerase chain reaction (RT-PCR) assay or an IHC assay. In certain other embodiments, no transformative step is involved and PD-L1 expression is assessed by, for example, reviewing a report of test results from a laboratory. In certain embodiments, the steps of the methods up to, and including, assessing PD-L1 expression provides an intermediate result that can be provided to a physician or other healthcare provider for use in selecting a suitable candidate for the combination therapy comprising (a) an anti-PD-1 antibody or an anti-PD-L1 antibody and (b) an anti-CTLA-4 antibody. In certain embodiments, the steps that provide the intermediate result is performed by a medical practitioner or someone acting under the direction of a medical practitioner. In other embodiments, these steps are performed by an independent laboratory or by an independent person such as a laboratory technician.

In certain embodiments of any of the present methods, the proportion of cells that express PD-L1 is assessed by performing an assay to determine the presence of PD-L1 RNA. In further embodiments, the presence of PD-L1 RNA is determined by RT-PCR, in situ hybridization or RNase protection. In other embodiments, the proportion of cells that express PD-L1 is assessed by performing an assay to determine the presence of PD-L1 polypeptide. In further embodiments, the presence of PD-L1 polypeptide is determined by immunohistochemistry (IHC), enzyme-linked immunosorbent assay (ELISA), in vivo imaging, or flow cytometry. In some embodiments, PD-L1 expression is assayed by IHC. In other embodiments of all of these methods, cell surface expression of PD-L1 is assayed using, e.g., IHC or in vivo imaging.

Imaging techniques have provided important tools in cancer research and treatment. Recent developments in molecular imaging systems, including positron emission tomography (PET), single-photon emission computed tomography (SPECT), fluorescence reflectance imaging (FRI), fluorescence-mediated tomography (FMT), bioluminescence imaging (BLI), laser-scanning confocal microscopy (LSCM) and multiphoton microscopy (MPM), will likely herald even greater use of these techniques in cancer research. Some of these molecular imaging systems allow clinicians to not only see where a tumor is located in the body, but also to visualize the expression and activity of specific molecules, cells, and biological processes that influence tumor behavior and/or responsiveness to therapeutic drugs (Condeelis and Weissleder, “In vivo imaging in cancer,” Cold Spring Harb. Perspect. Biol. 2(12):a003848 (2010)). Antibody specificity, coupled with the sensitivity and resolution of PET, makes immunoPET imaging particularly attractive for monitoring and assaying expression of antigens in tissue samples (McCabe and Wu, “Positive progress in immunoPET—not just a coincidence,” Cancer Biother. Radiopharm. 25(3):253-61 (2010); Olafsen et al., “ImmunoPET imaging of B-cell lymphoma using 124I-anti-CD20 scFv dimers (diabodies),” Protein Eng. Des. Sel. 23(4):243-9 (2010)). In certain embodiments of any of the present methods, PD-L1 expression is assayed by immunoPET imaging. In certain embodiments of any of the present methods, the proportion of cells in a test tissue sample that express PD-L1 is assessed by performing an assay to determine the presence of PD-L1 polypeptide on the surface of cells in the test tissue sample. In certain embodiments, the test tissue sample is a FFPE tissue sample. In other embodiments, the presence of PD-L1 polypeptide is determined by IHC assay. In further embodiments, the IHC assay is performed using an automated process. In some embodiments, the IHC assay is performed using an anti-PD-L1 monoclonal antibody to bind to the PD-L1 polypeptide. In certain embodiments, the anti-PD-L1 monoclonal antibody is selected from the group consisting of 28-8, 28-1, 28-12, 29-8, 5H1, and any combination thereof. See WO/2013/173223, which is incorporated by reference herein in its entirety.

In one embodiment of the present methods, an automated IHC method is used to assay the expression of PD-L1 on the surface of cells in FFPE tissue specimens, e.g., a tissue sample taken from a tumor derived from a NSCLC. The presence of human PD-L1 antigen can be measured in a test tissue sample by contacting the test sample, and a negative control sample (e.g., normal tissue), with a monoclonal antibody that specifically binds to human PD-L1, under conditions that allow for formation of a complex between the antibody or portion thereof and human PD-L1. In certain embodiments, the test and control tissue samples are FFPE samples. The formation of a complex is then detected, wherein a difference in complex formation between the test sample and the negative control sample is indicative of the presence of human PD-L1 antigen in the sample. Various methods are used to quantify PD-L1 expression.

In a particular embodiment, the automated IHC method comprises: (a) deparaffinizing and rehydrating mounted tissue sections in an autostainer; (b) retrieving antigen using a decloaking chamber and pH 6 buffer, heated to 110° C. for 10 min; (c) setting up reagents on an autostainer; and (d) running the autostainer to include steps of neutralizing endogenous peroxidase in the tissue specimen; blocking non-specific protein-binding sites on the slides; incubating the slides with primary antibody; incubating with a post primary blocking agent; incubating with NovoLink Polymer; adding a chromogen substrate and developing; and counterstaining with hematoxylin.

For assessing PD-L1 expression in tumor tissue samples, a pathologist examines the number of membrane PD-L1 tumor cells in each field under a microscope and mentally estimates the percentage of cells that are positive, then averages them to come to the final percentage. The different staining intensities are defined as 0/negative, 1+/weak, 2+/moderate, and 3+/strong. Typically, percentage values are first assigned to the 0 and 3+ buckets, and then the intermediate 1+ and 2+ intensities are considered. For highly heterogeneous tissues, the specimen is divided into zones, and each zone is scored separately and then combined into a single set of percentage values. The percentages of negative and positive cells for the different staining intensities are determined from each area and a median value is given to each zone. A final percentage value is given to the tissue for each staining intensity category: negative, 1+, 2+, and 3+. The sum of all staining intensities needs to be 100%. In one embodiment, the threshold number of cells that needs to be PD-L1 positive is at least about 100, at least about 125, at least about 150, at least about 175, or at least about 200 cells. In certain embodiments, the threshold number or cells that needs to be PD-L1 positive is at least about 100 cells.

Staining is also assessed in tumor-infiltrating inflammatory cells such as macrophages and lymphocytes. In most cases macrophages serve as an internal positive control since staining is observed in a large proportion of macrophages. While not required to stain with 3+ intensity, an absence of staining of macrophages should be taken into account to rule out any technical failure. Macrophages and lymphocytes are assessed for plasma membrane staining and only recorded for all samples as being positive or negative for each cell category. Staining is also characterized according to an outside/inside tumor immune cell designation. “Inside” means the immune cell is within the tumor tissue and/or on the boundaries of the tumor region without being physically intercalated among the tumor cells. “Outside” means that there is no physical association with the tumor, the immune cells being found in the periphery associated with connective or any associated adjacent tissue.

In certain embodiments of these scoring methods, the samples are scored by two pathologists operating independently, and the scores are subsequently consolidated. In certain other embodiments, the identification of positive and negative cells is scored using appropriate software.

A histoscore is used as a more quantitative measure of the IHC data. The histoscore is calculated as follows:

Histoscore=[(% tumor×1(low intensity))+(% tumor×2(medium intensity))+(% tumor×3(high intensity)]

To determine the histoscore, the pathologist estimates the percentage of stained cells in each intensity category within a specimen. Because expression of most biomarkers is heterogeneous the histoscore is a truer representation of the overall expression. The final histoscore range is 0 (no expression) to 300 (maximum expression).

An alternative means of quantifying PD-L1 expression in a test tissue sample IHC is to determine the adjusted inflammation score (AIS) score defined as the density of inflammation multiplied by the percent PD-L1 expression by tumor-infiltrating inflammatory cells (Taube et al., “Colocalization of inflammatory response with B7-hl expression in human melanocytic lesions supports an adaptive resistance mechanism of immune escape,” Sci. Transl. Med. 4(127):127ra37 (2012)).

In one embodiment, the PD-L1 expression level of a tumor is at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 75%, at least about 80%, at least about 85%, at least about 90%, at least about 95%, or about 100%. In another embodiment, the PD-L1 status of a tumor is at least about 1%. In other embodiments, the PD-L1 status of the subject is at least about 5%. In a certain embodiment, the PD-L1 status of a tumor is at least about 10%. In one embodiment, the PD-L1 status of the tumor is at least about 25%. In a particular embodiment, the PD-L1 status of the tumor is at least about 50%.

“PD-L1 positive” as used herein can be interchangeably used with “PD-L1 expression of at least about 1%”. In one embodiment, the PD-L1 positive tumors can thus have at least about 1%, at least about 2%, at least about 5%, at least about 10%, at least about 20%, at least about 25%, at least about 30%, at least about 40%, at least about 50%, at least about 60%, at least about 70%, at least about 75%, at least about 80%, at least about 85%, at least about 90%, at least about 95%, or about 100% of the tumor cells expressing PD-L1 as measured by an automated IHC. In certain embodiments, “PD-L1 positive” means that there are at least 100 cells that express PD-L1 on the surface of the cells.

In one embodiment, a tumor derived from a NSCLC that is PD-L1 positive and that has a high TMB has a greater likelihood of response to a combination therapy with (a) an anti-PD-1 antibody or an anti-PD-L1 antibody and (b) an anti-CTLA-4 antibody than a tumor with only high TMB, only PD-L1 positive expression, or neither. In one embodiment, the tumor derived from a NSCLC has at least about 1%, about 5%, about 10%, about 15%, about 20%, about 25%, about 30%, about 35%, about 40%, about 45%, or about 50% PD-L1 expression. In a particular embodiment, a tumor derived from a NSCLC with ≥50% PD-L1 expression and a high TMB status is more likely to respond to a combination therapy with (a) an anti-PD-1 antibody or an anti-PD-L1 antibody and (b) an anti-CTLA-4 antibody than a tumor with only high TMB, only ≥50% PD-L1 expression, or neither.

In certain embodiments, the tumor in the subject suitable for the immunotherapy, e.g., a combination therapy with (a) an anti-PD-1 antibody or an anti-PD-L1 antibody and (b) an anti-CTLA-4 antibody, in this disclosure does not express PD-L1 (less than 1%, less than 2%, less than 3%, less than 4%, or less than 5% membranous PD-L1). In some embodiments, the methods of the present disclosure are irrelevant to the PD-L1 expression.

### MSI Status

TMB status can be used alone or in combination with other factors, e.g., MSI status, as a means to predict the responsiveness of a tumor derived from a NSCLC to a combination therapy with (a) an anti-PD-1 antibody or an anti-PD-L1 antibody and (b) an anti-CTLA-4 antibody. In one embodiment, the MSI status is part of the TMB status. In other embodiments, the MSI status is measured separately from the TMB status.

Microsatellite instability (MSI) is the condition of genetic hypermutability that results from impaired DNA mismatch repair (MMR). The presence of MSI represents phenotypic evidence that MMR is not functioning normally. In most cases, the genetic basis for instability in MSI tumors is an inherited germline alteration in any one of the five human MMR genes: MSH2, MLH1, MSH6, PMS2, and PMS1. In certain embodiments, the tumor derived from a NSCLC (e.g., colon tumor) has a high degree of microsatellite instability (MSI-H) and has at least one mutation in genes MSH2, MLH1, MSH6, PMS2, or PMS1. In other embodiments, subjects receiving tumor treatment within a control group have no microsatellite instability (MSS or MSI stable) and has no mutation in genes MSH2, MLH1, MSH6, PMS2, and PMS1.

In one embodiment, the subject suitable for the combination therapy with (a) an anti-PD-1 antibody or an anti-PD-L1 antibody and (b) an anti-CTLA-4 antibody has a high TMB status and a MSI-H tumor derived from a NSCLC. As used herein, MSI-H tumors mean tumors having greater than at least about 30% of unstable MSI biomarkers. In some embodiments, the tumor derived from a NSCLC is MSI-H when a germline alteration is detected in at least two, at least three, at least four, or at least five MMR genes. In other embodiments, the tumor derived from a NSCLC is MSI-H when a germline alteration is detected in at least 30% of five or more MMR genes. In some embodiments, a germline alternation in MMR genes is measured by a polymerase chain reaction. In other embodiments, the tumor derived from a NCSLC is MSI-H when at least one protein encoded by DNA MMR genes is not detected in the tumor. In some embodiments, the at least one protein encoded by DNA MMR genes is detected by an immunohistochemistry.

### Treatment Methods of the Disclosure

The present disclosure is directed to a method for treating a subject afflicted with a tumor derived from a NSCLC comprising administering to the subject an effective amount of (a) an anti-PD-1 antibody or an anti-PD-L1 antibody and (b) an anti-CTLA-4 antibody, wherein the tumor has a high TMB status. In certain embodiments, the tumor has a TMB status of at least about 10 mutations per megabase. In some embodiments, the method further comprises measuring the TMB status of a biological sample obtained from the subject prior to the administering.

Certain cancer types have a higher frequency of mutations and, thus, have a high TMB, including lung cancer. (Alexandrov et al., Nature (2013) 500:415-421.) In one embodiment, the NSCLC has a squamous histology. In another embodiment, the NSCLC has a non-squamous histology.

The methods of treatment disclosed herein can provide an improved clinical response and/or clinical benefit for subjects afflicted with a tumor derived from a NSCLC and, in particular, subjects having a tumor with a high TMB. High TMB can be related to neoantigen burden, i.e., the number of neoantigens and T-cell reactivity and, thus, an immune-mediated anti-tumor response. Accordingly, high TMB is a factor that can be used, alone or in combination with other factors, to identity tumors (and patients having such tumors) more likely to benefit from therapy with (a) an anti-PD-1 antibody or an anti-PD-L1 antibody and (b) an anti-CTLA-4 antibody, e.g., as compared to current standard of care therapies.

In one embodiment, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the administration. In another embodiment, the subject exhibits an overall survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the administration. In yet another embodiment, the subject exhibits an objective response rate of at least about 30%, about 35%, about 40%, about 45%, about 50%, about 55%, about 60%, about 65%, about 70%, about 75%, about 80%, about 85%, about 90%, about 95%, or about 100%.

### Anti-PD-1 Anti-PD-L1 Anti-CTLA-4 Treatment

Certain aspects of the present disclosure are directed to a method for treating a subject afflicted with a tumor derived from a NSCLC, wherein the tumor has a high TMB status, e.g., a TMB of at least about 10 mutations per megabase of genes examined, comprising administering to the subject (a) an anti-PD-1 or an anti-PD-L1 antibody and (b) an anti-CTLA-4 antibody. The method can further comprise measuring the TMB status of a biological sample obtained from the subject. Additionally, the disclosure contemplates administering (a) an anti-PD-1 antibody or an anti-PD-L1 antibody and (b) an anti-CTLA-4 antibody to a subject identified as suitable for such therapy, e.g., based on measurement of a high TMB, e.g., at least about 10 mutations per megabase of genes examined.

In one embodiment, the anti-PD-1 antibody or antigen-binding portion thereof cross-competes with nivolumab for binding to human PD-1. In another embodiment, the anti-PD-1 antibody or antigen-binding portion thereof binds to the same epitope as nivolumab. In a particular embodiment, the anti-PD-1 antibody is nivolumab. In another particular embodiment, the anti-PD-1 antibody is pembrolizumab. Additional anti-PD-1 antibodies are described elsewhere herein. In other embodiments, an anti-PD-L1 antibody or antigen-binding portion thereof useful for the methods of the disclosure is described elsewhere herein.

In some embodiments, the anti-PD-1 antibody or an anti-PD-L1 antibody or antigen-binding portion thereof is a chimeric antibody, a humanized antibody, a human antibody, or an antigen-binding portion thereof. In other embodiments, the anti-PD-1 antibody or antigen-binding portion thereof or an anti-PD-L1 antibody or antigen-binding portion thereof comprises a heavy chain constant region of a human IgG1 isotype or a human IgG4 isotype.

### Anti-PD-1 Antibodies Useful for the Disclosure

Anti-PD-1 antibodies that are known in the art can be used in the presently described compositions and methods. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1×10⁻⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-7 production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some embodiments, at least five, of the preceding characteristics.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

In some embodiments, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; also known as toripalimab; see Si-Yang Liu et al., J. Hematol. Oncol. 10:136 (2017)), BGB-A317 (Beigene; also known as tislelizumab; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), BCD-100 (Biocad; Kaplon et al., mAbs 10(2):183-203 (2018), and IBI308 (Innovent; see WO 2017/024465, WO 2017/025016, WO 2017/132825, and WO 2017/133540).

In one embodiment, the anti-PD-1 antibody is nivolumab. Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

In another embodiment, the anti-PD-1 antibody is pembrolizumab. Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587.

Anti-PD-1 antibodies usable in the disclosed compositions and methods also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some embodiments, the anti-PD-1 antibody binds the same epitope as any of the anti-PD-1 antibodies described herein, e.g., nivolumab. The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain embodiments, the antibodies that cross-compete for binding to human PD-1 with, or bind to the same epitope region of human PD-1 antibody, nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-1 antibodies usable in the compositions and methods of the disclosed disclosure also include antigen-binding portions of the above antibodies. It has been amply demonstrated that the antigen-binding function of an antibody can be performed by fragments of a full-length antibody.

Anti-PD-1 antibodies suitable for use in the disclosed compositions and methods are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain embodiments, the anti-PD-1 antibody or antigen-binding portion thereof cross-competes with nivolumab for binding to human PD-1.

In some embodiments, the anti-PD-1 antibody is administered at a dose ranging from 0.1 mg/kg to 20.0 mg/kg body weight once every 2, 3, 4, 5, 6, 7, or 8 weeks, e.g., 0.1 mg/kg to 10.0 mg/kg body weight once every 2, 3, or 4 weeks. In other embodiments, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 2 weeks. In other embodiments, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 3 weeks. In one embodiment, the anti-PD-1 antibody is administered at a dose of about 5 mg/kg body weight about once every 3 weeks. In another embodiment, the anti-PD-1 antibody, e.g., nivolumab, is administered at a dose of about 3 mg/kg body weight about once every 2 weeks. In other embodiments, the anti-PD-1 antibody, e.g., pembrolizumab, is administered at a dose of about 2 mg/kg body weight about once every 3 weeks.

The anti-PD-1 antibody useful for the present disclosure can be administered as a flat dose. In some embodiments, the anti-PD-1 antibody is administered at a flat dose of from about 100 to about 1000 mg, from about 100 mg to about 900 mg, from about 100 mg to about 800 mg, from about 100 mg to about 700 mg, from about 100 mg to about 600 mg, from about 100 mg to about 500 mg, from about 200 mg to about 1000 mg, from about 200 mg to about 900 mg, from about 200 mg to about 800 mg, from about 200 mg to about 700 mg, from about 200 mg to about 600 mg, from about 200 mg to about 500 mg, from about 200 mg to about 480 mg, or from about 240 mg to about 480 mg, In one embodiment, the anti-PD-1 antibody is administered as a flat dose of at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg, at least about 540 mg, at least about 550 mg, at least about 560 mg, at least about 580 mg, at least about 600 mg, at least about 620 mg, at least about 640 mg, at least about 660 mg, at least about 680 mg, at least about 700 mg, or at least about 720 mg at a dosing interval of about 1, 2, 3, 4, 5, 6, 7, 8, 9, or 10 weeks. In another embodiments, the anti-PD-1 antibody is administered as a flat dose of about 200 mg to about 800 mg, about 200 mg to about 700 mg, about 200 mg to about 600 mg, about 200 mg to about 500 mg, at a dosing interval of about 1, 2, 3, or 4 weeks.

In some embodiments, the anti-PD-1 antibody is administered as a flat dose of about 200 mg at about once every 3 weeks. In other embodiments, the anti-PD-1 antibody is administered as a flat dose of about 200 mg at about once every 2 weeks. In other embodiments, the anti-PD-1 antibody is administered as a flat dose of about 240 mg at about once every 2 weeks. In certain embodiments, the anti-PD-1 antibody is administered as a flat dose of about 480 mg at about once every 4 weeks.

In some embodiments, nivolumab is administered at a flat dose of about 240 mg once about every 2 weeks. In some embodiments, nivolumab is administered at a flat dose of about 240 mg once about every 3 weeks. In some embodiments, nivolumab is administered at a flat dose of about 360 mg once about every 3 weeks. In some embodiments, nivolumab is administered at a flat dose of about 480 mg once about every 4 weeks.

In some embodiments, pembrolizumab is administered at a flat dose of about 200 mg once about every 2 weeks. In some embodiments, pembrolizumab is administered at a flat dose of about 200 mg once about every 3 weeks. In some embodiments, pembrolizumab is administered at a flat dose of about 400 mg once about every 4 weeks.

### Anti-PD-L1 Antibodies Useful for the Disclosure

In certain embodiments, an anti-PD-L1 antibody is substituted for the anti-PD-1 antibody in any of the methods disclosed herein. Anti-PD-L1 antibodies that are known in the art can be used in the compositions and methods of the present disclosure. Examples of anti-PD-L1 antibodies useful in the compositions and methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-7 production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include monoclonal antibodies that bind specifically to human PD-L1 and exhibit at least one, in some embodiments, at least five, of the preceding characteristics.

In certain embodiments, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., JCO 36 (15suppl):TPS3113 (2018)), and CK-301 (Checkpoint Therapeutics; see Gorelik et al., AACR:Abstract 4606 (April 2016)).

In certain embodiments, the PD-L1 antibody is atezolizumab (TECENTRIQ®). Atezolizumab is a fully humanized IgG1 monoclonal anti-PD-L1 antibody.

In certain embodiments, the PD-L1 antibody is durvalumab (IVIFINZI™). Durvalumab is a human IgG1 kappa monoclonal anti-PD-L1 antibody.

In certain embodiments, the PD-L1 antibody is avelumab (BAVENCIO®). Avelumab is a human IgG1 lambda monoclonal anti-PD-L1 antibody.

Anti-PD-L1 antibodies usable in the disclosed compositions and methods also include isolated antibodies that bind specifically to human PD-L1 and cross-compete for binding to human PD-L1 with any anti-PD-L1 antibody disclosed herein, e.g., atezolizumab, durvalumab, and/or avelumab. In some embodiments, the anti-PD-L1 antibody binds the same epitope as any of the anti-PD-L1 antibodies described herein, e.g., atezolizumab, durvalumab, and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., atezolizumab and/or avelumab, by virtue of their binding to the same epitope region of PD-L1. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In certain embodiments, the antibodies that cross-compete for binding to human PD-L1 with, or bind to the same epitope region of human PD-L1 antibody as, atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies usable in the compositions and methods of the disclosed disclosure also include antigen-binding portions of the above antibodies. It has been amply demonstrated that the antigen-binding function of an antibody can be performed by fragments of a full-length antibody.

Anti-PD-L1 antibodies suitable for use in the disclosed compositions and methods are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain embodiments, the anti-PD-L1 antibody or antigen-binding portion thereof cross-competes with atezolizumab, durvalumab, and/or avelumab for binding to human PD-L1.

The anti-PD-L1 antibody useful for the present disclosure can be any PD-L1 antibody that specifically binds to PD-L1, e.g., antibodies that cross-compete with durvalumab, avelumab, or atezolizumab for binding to human PD-1, e.g., an antibody that binds to the same epitope as durvalumab, avelumab, or atezolizumab. In a particular embodiment, the anti-PD-L1 antibody is durvalumab. In other embodiments, the anti-PD-L1 antibody is avelumab. In some embodiments, the anti-PD-L1 antibody is atezolizumab.

In some embodiments, the anti-PD-L1 antibody is administered at a dose ranging from about 0.1 mg/kg to about 20.0 mg/kg body weight, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, about once every 2, 3, 4, 5, 6, 7, or 8 weeks.

In some embodiments, the anti-PD-L1 antibody is administered at a dose of about 15 mg/kg body weight at about once every 3 weeks. In other embodiments, the anti-PD-L1 antibody is administered at a dose of about 10 mg/kg body weight at about once every 2 weeks.

In other embodiments, the anti-PD-L1 antibody useful for the present disclosure is a flat dose. In some embodiments, the anti-PD-L1 antibody is administered as a flat dose of from about 200 mg to about 1600 mg, about 200 mg to about 1500 mg, about 200 mg to about 1400 mg, about 200 mg to about 1300 mg, about 200 mg to about 1200 mg, about 200 mg to about 1100 mg, about 200 mg to about 1000 mg, about 200 mg to about 900 mg, about 200 mg to about 800 mg, about 200 mg to about 700 mg, about 200 mg to about 600 mg, about 700 mg to about 1300 mg, about 800 mg to about 1200 mg, about 700 mg to about 900 mg, or about 1100 mg to about 1300 mg. In some embodiments, the anti-PD-L1 antibody is administered as a flat dose of at least about 240 mg, at least about 300 mg, at least about 320 mg, at least about 400 mg, at least about 480 mg, at least about 500 mg, at least about 560 mg, at least about 600 mg, at least about 640 mg, at least about 700 mg, at least 720 mg, at least about 800 mg, at least about 840 mg, at least about 880 mg, at least about 900 mg, at least 960 mg, at least about 1000 mg, at least about 1040 mg, at least about 1100 mg, at least about 1120 mg, at least about 1200 mg, at least about 1280 mg, at least about 1300 mg, at least about 1360 mg, or at least about 1400 mg, at a dosing interval of about 1, 2, 3, or 4 weeks. In some embodiments, the anti-PD-L1 antibody is administered as a flat dose of about 1200 mg at about once every 3 weeks. In other embodiments, the anti-PD-L1 antibody is administered as a flat dose of about 800 mg at about once every 2 weeks. In other embodiments, the anti-PD-L1 antibody is administered as a flat dose of about 840 mg at about once every 2 weeks.

In some embodiments, atezolizumab is administered as a flat dose of about 1200 mg once about every 3 weeks. In some embodiments, atezolizumab is administered as a flat dose of about 800 mg once about every 2 weeks. In some embodiments, atezolizumab is administered as a flat dose of about 840 mg once about every 2 weeks.

In some embodiments, avelumab is administered as a flat dose of about 800 mg once about every 2 weeks.

In some embodiments, durvalumab is administered at a dose of about 10 mg/kg once about every 2 weeks. In some embodiments, durvalumab is administered as a flat dose of about 800 mg/kg once about every 2 weeks. In some embodiments, durvalumab is administered as a flat dose of about 1200 mg/kg once about every 3 weeks.

### Anti-CTLA-4 Antibodies

Anti-CTLA-4 antibodies that are known in the art can be used in the compositions and methods of the present disclosure. Anti-CTLA-4 antibodies of the instant disclosure bind to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

Human monoclonal antibodies that bind specifically to CTLA-4 with high affinity have been disclosed in U.S. Pat. No. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety. The anti-CTLA-4 human monoclonal antibodies disclosed in U.S. Pat. No. 6,984,720 have been demonstrated to exhibit one or more of the following characteristics: (a) binds specifically to human CTLA-4 with a binding affinity reflected by an equilibrium association constant (Kₐ) of at least about 107 M⁻¹, or about 10⁹ M⁻¹, or about 10¹⁰ M⁻¹ to 10¹¹ M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (k_{d}) of at least about 10³, about 10⁴, or about 10⁵ m⁻¹ s⁻¹; and (d) inhibits the binding of CTLA-4 to B7-1 (CD80) and B7-2 (CD86). Anti-CTLA-4 antibodies useful for the present disclosure include monoclonal antibodies that bind specifically to human CTLA-4 and exhibit at least one, at least two, or at least three of the preceding characteristics.

In certain embodiments, the CTLA-4 antibody is selected from the group consisting of ipilimumab (also known as YERVOY®, MDX-010, 10D1; see U.S. Pat. No. 6,984,720), MK-1308 (Merck), AGEN-1884 (Agenus Inc.; see WO 2016/196237), and tremelimumab (AstraZeneca; also known as ticilimumab, CP-675,206; see WO 2000/037504 and Ribas, Update Cancer Ther. 2(3): 133-39 (2007)). In particular embodiments, the anti-CTLA-4 antibody is ipilimumab.

In particular embodiments, the CTLA-4 antibody is ipilimumab for use in the compositions and methods disclosed herein. Ipilimumab is a fully human, IgG1 monoclonal antibody that blocks the binding of CTLA-4 to its B7 ligands, thereby stimulating T cell activation and improving overall survival (OS) in patients with advanced melanoma.

In particular embodiments, the CTLA-4 antibody is tremelimumab.

In particular embodiments, the CTLA-4 antibody is MK-1308.

In particular embodiments, the CTLA-4 antibody is AGEN-1884.

Anti-CTLA-4 antibodies usable in the disclosed compositions and methods also include isolated antibodies that bind specifically to human CTLA-4 and cross-compete for binding to human CTLA-4 with any anti-CTLA-4 antibody disclosed herein, e.g., ipilimumab and/or tremelimumab. In some embodiments, the anti-CTLA-4 antibody binds the same epitope as any of the anti-CTLA-4 antibodies described herein, e.g., ipilimumab and/or tremelimumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., ipilimumab and/or tremelimumab, by virtue of their binding to the same epitope region of CTLA-4. Cross-competing antibodies can be readily identified based on their ability to cross-compete with ipilimumab and/or tremelimumab in standard CTLA-4 binding assays such as Biacore analysis, ELISA assays or flow cytometry (see, e.g., WO 2013/173223).

In certain embodiments, the antibodies that cross-compete for binding to human CTLA-4 with, or bind to the same epitope region of human CTLA-4 antibody as, ipilimumab and/or tremelimumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-CTLA-4 antibodies usable in the compositions and methods of the disclosed disclosure also include antigen-binding portions of the above antibodies. It has been amply demonstrated that the antigen-binding function of an antibody can be performed by fragments of a full-length antibody.

Anti-CTLA-4 antibodies suitable for use in the disclosed methods or compositions are antibodies that bind to CTLA-4 with high specificity and affinity, block the activity of CTLA-4, and disrupt the interaction of CTLA-4 with a human B7 receptor. In any of the compositions or methods disclosed herein, an anti-CTLA-4 “antibody” includes an antigen-binding portion or fragment that binds to CTLA-4 and exhibits the functional properties similar to those of whole antibodies in inhibiting the interaction of CTLA-4 with a human B7 receptor and up-regulating the immune system. In certain embodiments, the anti-CTLA-4 antibody or antigen-binding portion thereof cross-competes with ipilimumab and/or tremelimumab for binding to human CTLA-4.

In some embodiments, the anti-CTLA-4 antibody or antigen-binding portion thereof is administered at a dose ranging from 0.1 mg/kg to 10.0 mg/kg body weight once every 2, 3, 4, 5, 6, 7, or 8 weeks. In some embodiments, the anti-CTLA-4 antibody or antigen-binding portion thereof is administered at a dose of 1 mg/kg or 3 mg/kg body weight once every 3, 4, 5, or 6 weeks. In one embodiment, the anti-CTLA-4 antibody or antigen-binding portion thereof is administered at a dose of 3 mg/kg body weight once every 2 weeks. In another embodiment, the anti-PD-1 antibody or antigen-binding portion thereof is administered at a dose of 1 mg/kg body weight once every 6 weeks.

In some embodiments, the anti-CTLA-4 antibody or antigen-binding portion thereof is administered as a flat dose. In some embodiments, the anti-CTLA-4 antibody is administered at a flat dose of from about 10 to about 1000 mg, from about 10 mg to about 900 mg, from about 10 mg to about 800 mg, from about 10 mg to about 700 mg, from about 10 mg to about 600 mg, from about 10 mg to about 500 mg, from about 100 mg to about 1000 mg, from about 100 mg to about 900 mg, from about 100 mg to about 800 mg, from about 100 mg to about 700 mg, from about 100 mg to about 100 mg, from about 100 mg to about 500 mg, from about 100 mg to about 480 mg, or from about 240 mg to about 480 mg. In one embodiment, the anti-CTLA-4 antibody or antigen-binding portion thereof is administered as a flat dose of at least about 60 mg, at least about 80 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg at least about 540 mg, at least about 550 mg, at least about 560 mg, at least about 580 mg, at least about 600 mg, at least about 620 mg, at least about 640 mg, at least about 660 mg, at least about 680 mg, at least about 700 mg, or at least about 720 mg. In another embodiment, the anti-CTLA-4 antibody or antigen-binding portion thereof is administered as a flat dose about once every 1, 2, 3, 4, 5, 6, 7, or 8 weeks.

In some embodiments, ipilimumab is administered at a dose of about 3 mg/kg once about every 3 weeks. In some embodiments, ipilimumab is administered at a dose of about 10 mg/kg once about every 3 weeks. In some embodiments, ipilimumab is administered at a dose of about 10 mg/kg once about every 12 weeks. In some embodiments, the ipilimumab is administered for four doses.

### Cytokines

In some embodiments, the method comprises treating a subject afflicted with a tumor derived from a NSCLC comprising administering (a) an anti-PD-1 antibody or an anti-PD-L1 antibody, (b) an anti-CTLA-4 antibody, and (c) a cytokine, wherein the tumor has a high TMB status, e.g., wherein the tumor has a TMB stats of at least about 10 mutations per megabase of genes examined. The cytokine can be any cytokine or variant thereof known in the art. In some embodiments, the cytokine is selected from the group consisting of interleukin-2 (IL-2), IL-1β, IL-6, TNF-α, RANTES, monocyte chemoattractant protein (MCP-1), monocyte inflammatory protein (MIP-1α and MIP-1β), IL-8, lymphotactin, fractalkine, IL-1, IL-4, IL-10, IL-11, IL-13, LIF, interferon-alpha, TGF-beta, and any combination thereof. In some embodiments, the cytokine is a CD122 agonist. In certain embodiments, the cytokine comprises IL-2 or a variant thereof.

In some embodiments, the cytokine comprises one or more amino acid substitution, deletion, or insertion relative to the wild-type cytokine amino acid sequence. In some embodiments, the cytokine comprises an amino acid sequence having at least 1, at least 2, at least 3, at least 4, at least 5, at least 6, at least 7, at least 8, at least 9, or at least 10 amino acids substituted relative to the amino acid sequence of the wild-type cytokine.

In some embodiments, the cytokine is modified, e.g., to increase activity and/or half-life. In certain embodiments, the cytokine is modified through fusion of a heterologous moiety to the cytokine. The heterologous moiety can be any structure including a polypeptide, a polymer, a small molecule, a nucleotide, or a fragment or analog thereof. In certain embodiments, the heterologous moiety comprises a polypeptide. In some embodiments, the heterologous moiety comprises albumin or a fragment thereof, albumin-binding polypeptide (ABP), XTEN, Fc, PAS, the C-terminal peptide (CTP) of the R subunit of human chorionic gonadotropin, or any combination thereof.

In certain embodiments, the cytokine is modified through fusion of the cytokine with a polymer. In some embodiments, the polymer comprises polyethylene glycol (PEG), polypropylene glycol (PPG), hydroxyethyl starch (HES), or any combination thereof. “PEG” or “polyethylene glycol,” as used herein, is meant to encompass any water-soluble poly(ethylene oxide). Unless otherwise indicated, a “PEG polymer” or a polyethylene glycol is one in which substantially all (preferably all) monomeric subunits are ethylene oxide subunits, though, the polymer may contain distinct end capping moieties or functional groups, e.g., for conjugation. PEG polymers for use in the present disclosure will comprise one of the two following structures: “—(CH₂CH₂0)ₙ₋ₙ or “—(CH₂CH₂0)ₙ₋₁CH₂CH₂—,” depending upon whether or not the terminal oxygen(s) has been displaced, e.g., during a synthetic transformation. As stated above, for the PEG polymers, the variable (n) ranges from about 3 to 4000, and the terminal groups and architecture of the overall PEG can vary.

In some embodiments, the present disclosure is directed to methods of treating a subject afflicted with a tumor derived from a NSCLC comprising administering to the subject (a) an anti-PD-1 antibody or an anti-PD-L1 antibody, (b) an anti-CTLA-4 antibody, and (c) a CD122 agonist. In some embodiments, the method comprises administering to the subject (a) an anti-PD-1 antibody, (b) an anti-CTLA-4 antibody, and (c) a CD122 agonist. In other embodiments, the method comprises administering to the subject (a) an anti-PD-L1 antibody, (b) an anti-CTLA-4 antibody, and (c) a CD122 agonist. In some embodiments, the CD122 agonist comprises IL-2 or a variant thereof. In some embodiments, the CD122 agonist comprises an IL-2 variant having at least 1 amino acid substitution relative to wild-type IL-2. In some embodiments, the CD122 agonist comprises an IL-2 fused to a PEG. In some embodiments, the CD122 agonist comprises an IL-2 variant having at least 1 amino acid substitution relative to wild-type IL-2, wherein the IL-2 variant is fused to a PEG.

### Combination Therapies

In certain embodiments, the anti-PD-1 antibody, the anti-PD-L1 antibody, and/or the anti-CTLA-4 antibody are administered at a therapeutically effective amount. In some embodiments, the method comprises administering a therapeutically effective amount of anti-PD-1 antibody and an anti-CTLA-4 antibody. In other embodiments, the method comprises administering a therapeutically effective amount of anti-PD-L1 antibody and an anti-CTLA-4 antibody. Any anti-PD-1, anti-PD-L1, or anti-CTLA-4 antibody disclosed herein can be used in the method. In certain embodiments, the anti-PD-1 antibody comprises nivolumab. In some embodiments, the anti-PD-1 antibody comprises pembrolizumab. In some embodiments, the anti-PD-L1 antibody comprises atezolizumab. In some embodiments, the anti-PD-L1 antibody comprises durvalumab. In some embodiments, the anti-PD-L1 antibody comprises avelumab. In some embodiments, the anti-CTLA-4 antibody comprises ipilimumab. In some embodiments, the anti-CTLA-4 antibody comprises ipilimumab tremelimumab.

In some embodiments, the (a) anti-PD-1 antibody or the anti-PD-L1 antibody and the (b) anti-CTLA-4 antibody are each administered once about every 2 weeks, once about every 3 weeks, once about every 4 weeks, once about every 5 weeks, or once about every 6 weeks. In some embodiments, the anti-PD-1 antibody or the anti-PD-L1 antibody is administered once about every 2 weeks, once about every 3 weeks or once about every 4 weeks, and the anti-CTLA-4 antibody is administered once about every 6 weeks. In some embodiments, the anti-PD-1 antibody or anti-PD-L1 antibody is administered on the same day as the anti-CTLA-4 antibody. In some embodiments, the anti-PD-1 antibody or the anti-PD-L1 antibody is administered on a different day than the anti-CTLA-4 antibody.

In some embodiments, the anti-CTLA-4 antibody is administered at a dose ranging from about 0.1 mg/kg to about 20.0 mg/kg body weight once about every 2, 3, 4, 5, 6, 7, or 8 weeks. In some embodiments, the anti-CTLA-4 antibody is administered at a dose of about 0.1 mg/kg, about 0.3 mg/kg, about 0.6 mg/kg, about 0.9 mg/kg, about 1 mg/kg, about 3 mg/kg, about 6 mg/kg, about 9 mg/kg, about 10 mg/kg, about 12 mg/kg, about 15 mg/kg, about 18 mg/kg, or about 20 mg/kg. In certain embodiments, the anti-CTLA-4 antibody is administered at a dose of about 1 mg/kg once about every 4 weeks. In some embodiments, the anti-CTLA-4 antibody is administered at a dose of about 1 mg/kg once about every 6 weeks.

In some embodiments, the anti-CTLA-4 antibody is administered at a flat dose. In some embodiments, the anti-CTLA-4 antibody is administered at a flat dose ranging from at least about 40 mg to at least about 1600 mg. In some embodiments, the anti-CTLA-4 antibody is administered at a flat dose of at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, or at least about 200 mg. In some embodiments, the CTLA-4 antibody is administered at a flat dose of at least about 220 mg, at least about 230 mg, at least about 240 mg, at least about 250 mg, at least about 260 mg, at least about 270 mg, at least about 280 mg, at least about 290 mg, at least about 300 mg, at least about 320 mg, at least about 360 mg, at least about 400 mg, at least about 440 mg, at least about 480 mg, at least about 520 mg, at least about 560 mg, or at least about 600 mg. In some embodiments, the CTLA-4 antibody is administered at a flat dose of at least about 640 mg, at least about 720 mg, at least about 800 mg, at least about 880 mg, at least about 960 mg, at least about 1040 mg, at least about 1120 mg, at least about 1200 mg, at least about 1280 mg, at least about 1360 mg, at least about 1440 mg, or at least about 1600 mg. In some embodiments, the anti-CTLA-4 antibody is administered in a flat dose at least once about every 2, 3, 4, 5, 6, 7, or 8 weeks.

In certain embodiments, the anti-PD-1 antibody is administered at a dose of about 2 mg/kg once about every 3 weeks and the anti-CTLA-4 antibody is administered at a dose of about 1 mg/kg once about every 6 weeks. In some embodiments, the anti-PD-1 antibody is administered at a dose of about 3 mg/kg once about every 2 weeks and the anti-CTLA-4 antibody is administered at a dose of about 1 mg/kg once about every 6 weeks. In some embodiments, the anti-PD-1 antibody is administered at a dose of about 6 mg/kg once about every 4 weeks and the anti-CTLA-4 antibody is administered at a dose of about 1 mg/kg once about every 6 weeks.

In certain embodiments, the anti-PD-1 antibody is administered at a flat dose of about 200 mg once about every 3 weeks and the anti-CTLA-4 antibody is administered at a dose of about 1 mg/kg once about every 6 weeks. In some embodiments, the anti-PD-1 antibody is administered at a flat dose of about 200 mg once about every 2 weeks and the anti-CTLA-4 antibody is administered at a dose of about 1 mg/kg once about every 6 weeks. In some embodiments, the anti-PD-1 antibody is administered at a flat dose of about 240 mg once about every 2 weeks and the anti-CTLA-4 antibody is administered at a dose of about 1 mg/kg once about every 6 weeks. In some embodiments, the anti-PD-1 antibody is administered at a flat dose of about 480 mg once about every 4 weeks and the anti-CTLA-4 antibody is administered at a dose of about 1 mg/kg once about every 6 weeks.

In certain embodiments, the anti-PD-1 antibody is administered at a flat dose of about 200 mg once about every 3 weeks and the anti-CTLA-4 antibody is administered at a flat dose of about 80 mg once about every 6 weeks. In some embodiments, the anti-PD-1 antibody is administered at a flat dose of about 200 mg once about every 2 weeks and the anti-CTLA-4 antibody is administered at a dose of about 80 mg once about every 6 weeks. In some embodiments, the anti-PD-1 antibody is administered at a flat dose of about 240 mg once about every 2 weeks and the anti-CTLA-4 antibody is administered at a dose of about 80 mg once about every 6 weeks. In some embodiments, the anti-PD-1 antibody is administered at a flat dose of about 480 mg once about every 4 weeks and the anti-CTLA-4 antibody is administered at a dose of about 80 mg once about every 6 weeks.

In certain embodiments, the anti-PD-L1 antibody is administered at a dose of about 10 mg/kg once about every 2 weeks and the anti-CTLA-4 antibody is administered at a dose of about 1 mg/kg once about every 6 weeks. In some embodiments, the anti-PD-L1 antibody is administered at a dose of about 15 mg/kg once about every 3 weeks and the anti-CTLA-4 antibody is administered at a dose of about 1 mg/kg once about every 6 weeks.

In certain embodiments, the anti-PD-L1 antibody is administered at a flat dose of about 800 mg once about every 2 weeks and the anti-CTLA-4 antibody is administered at a dose of about 1 mg/kg once about every 6 weeks. In some embodiments, the anti-PD-L1 antibody is administered at a flat dose of about 1200 mg once about every 3 weeks and the anti-CTLA-4 antibody is administered at a dose of about 1 mg/kg once about every 6 weeks.

In certain embodiments, the anti-PD-L1 antibody is administered at a flat dose of about 800 mg once about every 2 weeks and the anti-CTLA-4 antibody is administered at a flat dose of about 80 mg once about every 6 weeks. In some embodiments, the anti-PD-L1 antibody is administered at a flat dose of about 1200 mg once about every 3 weeks and the anti-CTLA-4 antibody is administered at a dose of about 80 mg once about every 6 weeks.

In some embodiments, the anti-PD-1 antibody, e.g., nivolumab, is administered at a dose of about 3 mg/kg and the anti-CTLA-4 antibody is administered at a dose of about 1 mg/kg on the same day, once about every 3 weeks for 4 doses, then the anti-PD-1 antibody, e.g., nivolumab, is administered at a flat dose of 240 mg once about every 2 weeks or 480 mg once about every 4 weeks. In some embodiments, the anti-PD-1 antibody, e.g., nivolumab, is administered at a dose of about 1 mg/kg and the anti-CTLA-4 antibody is administered at a dose of about 3 mg/kg on the same day, once about every 3 weeks for 4 doses, then the anti-PD-1 antibody, e.g., nivolumab, is administered at a flat dose of 240 mg once about every 2 weeks or 480 mg once about every 4 weeks.

### NSCLC

NSCLC is the leading cause of cancer death in the U.S. and worldwide, exceeding breast, colon and prostate cancer combined. In the U.S., an estimated 228,190 new cases of lung and bronchial will be diagnosed in the U.S., and some 159,480 deaths will occur because of the disease (Siegel et al. (2014) CA Cancer J Clin 64(1):9-29). The majority of patients (approximately 78%) are diagnosed with advanced/recurrent or metastatic disease. Metastases to the adrenal gland from lung cancer are a common occurrence, with about 33% of patients having such metastases. NSCLC therapies have incrementally improved OS, but benefit has reached a plateau (median OS for late stage patients is just 1 year). Progression after 1L therapy occurred in nearly all of these subjects and the 5-year survival rate is only 3.6% in the refractory setting. From 2005 to 2009, the overall 5-year relative survival rate for lung cancer in the U.S. was 15.9% (NCCN GUIDELINES®, Version 3.2014—Non-Small Cell Lung Cancer, available at: www.nccn.org/professionals/physician_gls/pdf/nscl.pdf, last accessed May 14, 2014).

The present methods can treat an NSCLC tumor at any stage. In certain embodiments, the tumor is derived from an NSCLC of any stage. There are at least seven stages used for NSCLC: occult (hidden) stage, Stage 0 (carcinoma in situ), Stage I, Stage II, Stage IIIA, Stage IIIB, and Stage IV. In the occult stage, the cancer cannot be seen by imaging or bronchoscopy. In Stage 0, cancer cells are found in the lining of the airways.

In one embodiment, the present methods treat a Stage I non-squamous NSCLC. Stage I NSCLC is divided in Stage IA and IB. In Stage IA, the tumor is in the lung only and is 3 centimeters or smaller. In Stage IB, the cancer has not spread to the lymph nodes and one or more of the following is true: 1) the tumor is larger than 3 centimeters but not larger than 5 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus.

In another embodiment, the methods of the present disclosure treat a Stage II non-squamous NSCLC. Stage II NSCLC is divided into Stage IIA and IIB. In Stage IIA, the cancer has either spread to the lymph nodes or not. If the cancer has spread to the lymph nodes, then the cancer can only have spread to the lymph nodes on the same side of the chest as the tumor, the lymph nodes with cancer or within the lung or near the bronchus. and one or more of the following is true: 1) the tumor is not larger than 5 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. The tumor is also considered Stage IIA if the cancer has not spread to the lymph nodes and one or more of the following is true: 1) the tumor is larger than 5 centimeters but not larger than 7 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. In stage IIB, the cancer has either spread to the lymph nodes or not. If the cancer has spread to the lymph nodes, then the cancer can only have spread to the lymph nodes on the same side of the chest as the tumor, the lymph nodes with cancer are within the lung or near the bronchus and one or more of the following is true: 1) the tumor is larger than 5 centimeters but not larger than 7 centimeters; 2) the cancer has spread to the main bronchus and is at least 2 centimeters below where the trachea joins the bronchus; 3) cancer has spread to the innermost layer of the membrane that covers the lung; or 4) part of the lung has collapsed or developed pneumonitis (inflammation of the lung) in the area where the trachea joins the bronchus. The tumor is also considered Stage IIB if the cancer has not spread to the lymph nodes and one or more of the following is true: 1) the tumor is larger than 7 centimeters; 2) the cancer has spread to the main bronchus (and is at least 2 centimeters below where the trachea joins the bronchus), the chest wall, the diaphragm, or the nerve that controls the diaphragm; 3) cancer has spread to the membrane around the heart or lining the chest wall; 4) the whole lung has collapsed or developed pneumonitis (inflammation of the lung); or 5) there are one or more separate tumors in the same lobe of the lung.

In other embodiments, any methods of the present disclosure treat Stage III non-squamous NSCLC. Stage IIIA is divided into 3 sections. These 3 sections are based on 1) the size of the tumor; 2) where the tumor is found and 3) which (if any) lymph nodes have cancer. In the first type of Stage IIIA NSCLC, the cancer has spread to the lymph nodes on the same side of the chest as the tumor, and the lymph nodes with the cancer are near the sternum or where the bronchus enters the lung. Additionally: 1) the tumor can be any size; 2) part of the lung (where the trachea joins the bronchus) or the whole lung can have collapsed or developed pneumonitis (inflammation of the lung); 3) there can be one or more separate tumors in the same lobe of the lung; and 4) cancer can have spread to any of the following: a) main bronchus, but not the area where the trachea joins the bronchus, b) chest well, c) diaphragm and the nerve that controls it, d) membrane around the lung or lining the chest wall, e) membrane around the heart. In the second type of Stage IIIA NSCLC, the cancer has spread to the lymph nodes on the same side of the chest as the tumor, and the lymph nodes with the cancer are within the lung or near the bronchus. Additionally: 1) the tumor can be any size; 2) the whole lung can have collapsed or developed pneumonitis (inflammation of the lung); 3) there can be one or more separate tumors in the any of the lobes of the lung with cancer; and 4) cancer can have spread to any of the following: a) main bronchus, but not the area where the trachea joins the bronchus, b) chest well, c) diaphragm and the nerve that controls it, d) membrane around the lung or lining the chest wall, e) heart or the membrane around it, f) major blood vessels that lead to or from the heart, g) trachea, h) esophagus, i) nerve that controls the larynx (voice box), j) sternum (chest bone) or backbone, or k) carina (where the trachea joins the bronchi). In the third type of Stage IIIA NSCLC, the cancer has not spread to the lymph nodes, the tumor can be any size, and cancer has spread to any one of the following: a) heart, b) major blood vessels that lead to or from the heart, c) trachea, d) esophagus, e) nerve that controls the larynx (voice box), f) sternum (chest bone) or backbone, or g) carina (where the trachea joins the bronchi). Stage IIIB is divided into 2 sections depending on 1) the size of the tumor, 2) where the tumor is found, and 3) which lymph nodes have cancer. In the first type of Stage IIIB NSCLC, the cancer has spread to the lymph nodes on the opposite side of the chest as the tumor. Additionally, 1) the tumor can be any size; 2) part of the lung (where the trachea joins the bronchus) or the whole lung can have collapsed or developed pneumonitis (inflammation of the lung); 3) there can be one or more separate tumors in any of the lobs of the lung with cancer; and 4) cancer can have spread to any of the following: a) main bronchus, b) chest well, c) diaphragm and the nerve that controls it, d) membrane around the lung or lining the chest wall, e) heart or the membrane around it, f) major blood vessels that lead to or from the heart, g) trachea, h) esophagus, i) nerve that controls the larynx (voice box), j) sternum (chest bone) or backbone, or k) carina (where the trachea joins the bronchi). In the second type of Stage IIIB NSCLC, the cancer has spread to lymph nodes on the same side of the chest as the tumor. The lymph nodes with cancer are near the sternum (chest bone) or where the bronchus enters the lung. Additionally, 1) the tumor can be any size; 2) there can be separate tumors in different lobes of the same lung; and 3) cancer has spread to any of the following: a) heart, b) major blood vessels that lead to or from the heart, c) trachea, d) esophagus, e) nerve that controls the larynx (voice box), f) sternum (chest bone) or backbone, or g) carina (where the trachea joins the bronchi).

In some embodiments, the methods of the disclosure treat a Stage IV non-squamous NSCLC. In Stage IV NSCLC, the tumor can be any size and the cancer can have spread to the lymph nodes. One or more of the following is true in Stage IV NSCLC: 1) there are one or more tumors in both lungs; 2) cancer is found in the fluid around the lungs or heart; and 3) cancer has spread to other parts of the body, such as the brain, liver, adrenal glands, kidneys or bone.

In some embodiments, the subject has never smoked. In certain embodiments, the subject has formerly smoked. In one embodiment, the subject currently smokes. In certain embodiments, the subject has cancer cells that are squamous. In certain embodiments, the subject has cancer cells that are non-squamous.

### Standard-of-Care Therapies for Lung Cancer

In certain aspects of the present disclosure, the subject has received at least one prior therapy for the treatment of the tumor derived from the NSCLC. The at least one prior therapy can be any therapy known in the art for the treatment of NSCLC or a tumor derived therefrom. In particular, the at least one prior therapy can be a standard-of-care therapy for the treatment of NSCLC.

Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard-of-care treatments for a wide variety of cancers (see NCCN GUIDELINES® (2014), available at: www.nccn.org/professionals/physician_gls/f_guidelines.asp, last accessed May 14, 2014).

Surgery, radiation therapy (RT) and chemotherapy are the three modalities commonly used to treat NSCLC patients. As a class, NSCLCs are relatively insensitive to chemotherapy and RT, compared to small cell carcinoma. In general, for patients with Stage I or II disease, surgical resection provides the best chance for cure, with chemotherapy increasingly being used both pre-operatively and post-operatively. RT can also be used as adjuvant therapy for patients with resectable NSCLC, the primary local treatment, or as palliative therapy for patients with incurable NSCLC.

Patients with Stage IV disease who have a good performance status (PS) benefit from chemotherapy. Many drugs, including platinum agents (e.g., cisplatin, carboplatin), taxanes agents (e.g., paclitaxel, albumin-bound paclitaxel, docetaxel), vinorelbine, vinblastine, etoposide, pemetrexed and gemcitabine are useful for Stage IV NSCLC. Combinations using many of these drugs produce 1-year survival rates of 30% to 40% and are superior to single agents. Specific targeted therapies have also been developed for the treatment of advanced lung cancer. For example, bevacizumab (AVASTIN®) is a mAb that blocks vascular endothelial growth factor A (VEGF-A). Erlotinib (TARCEVA®) is a small-molecule TKI of epidermal growth factor receptor (EGFR). Crizotinib (XALKORI®) is a small-molecule TKI that targets ALK and MET, and is used to treat NSCLC in patients carrying the mutated ALK fusion gene. Cetuximab (ERBITUX®) is a mAb that targets EGFR.

There is a particular unmet need among patients who have squamous cell NSCLC (representing up to 25% of all NSCLC) as there are few treatment options after first line (1L) therapy. Single-agent chemotherapy is standard of care following progression with platinum-based doublet chemotherapy (Pt-doublet), resulting in median OS of approximately 7 months. Docetaxel remains the benchmark treatment in this line of therapy although erlotinib can also be used with less frequency. Pemetrexed has also been shown to produce clinically equivalent efficacy outcomes but with significantly fewer side effects compared with docetaxel in the second line (2L) treatment of patients with advanced NSCLC (Hanna et al. (2004) J Clin Oncol 22:1589-97). No therapy is currently approved for use in lung cancer beyond the third line (3L) setting. Pemetrexed and bevacizumab are not approved in squamous NSCLC, and molecularly targeted therapies have limited application. The unmet need in advanced lung cancer has been compounded by the recent failure of Oncothyreon and Merck KgaA's STIMUVAX® to improve OS in a phase 3 trial, inability of ArQule's and Daiichi Sankyo's c-Met kinase inhibitor, tivantinib, to meet survival endpoints, failure of Eli Lilly's ALIMTA® in combination with Roche's AVASTIN® to improve OS in a late-stage study, and Amgen's and Takeda Pharmaceutical's failure to meet clinical endpoints with the small-molecule VEGF-R antagonist, motesanib, in late-stage trials.

In certain embodiments, the at least one prior therapy comprises a standard-of-care therapy for the treatment of NSCLC or a tumor derived therefrom. In some embodiments, the at least one prior therapy comprises a surgery, a radiation therapy, a chemotherapy, an immunotherapy, or any combination thereof. In some embodiments, the at least one prior therapy comprises a chemotherapy. In some embodiments, the at least one prior therapy is selected from a therapy comprising administration of an anticancer agent selected from the group consisting of a platinum agent (e.g., cisplatin, carboplatin), a taxanes agent (e.g., paclitaxel, albumin-bound paclitaxel, docetaxel), vinorelbine, vinblastine, etoposide, pemetrexed, gemcitabine, bevacizumab (AVASTIN®), erlotinib (TARCEVA®), crizotinib (XALKORI®), cetuximab (ERBITUX®), and any combination thereof. In certain embodiments, the at least one prior therapy comprises a platinum-based doublet chemotherapy.

In some embodiments, the subject has experienced disease progression after the at least one prior therapy. In certain embodiments, the subject has received at least two prior therapies, at least three prior therapies, at least four prior therapies, or at least 5 prior therapies. In certain embodiments, the subject has received at least two prior therapies. In one embodiment, the subject has experienced disease progression after the at least two prior therapies. In certain embodiments, the at least two prior therapies comprises a first prior therapy and a second prior therapy, wherein the subject has experienced disease progression after the first prior therapy and/or the second prior therapy, and wherein the first prior therapy comprises a surgery, a radiation therapy, a chemotherapy, an immunotherapy, or any combination thereof, and wherein the second prior therapy comprises a surgery, a radiation therapy, a chemotherapy, an immunotherapy, or any combination thereof. In some embodiments, the first prior therapy comprises a platinum-based doublet chemotherapy, and the second prior therapy comprises a single-agent chemotherapy. In certain embodiments, the single-agent chemotherapy comprises docetaxel.

In some aspects of the present disclosure, the methods disclosed herein further comprise administering an additional anticancer therapy. The additional anticancer therapy can comprise any therapy known in the art for the treatment of an NSCLC or a tumor derived therefrom and/or any standard-of-care therapy, as disclosed herein. In some embodiments, the additional anticancer therapy comprises a surgery, a radiation therapy, a chemotherapy, an immunotherapy, or any combination thereof. In some embodiments, the additional anticancer therapy comprises a chemotherapy, including any chemotherapy disclosed herein. In some embodiment, the additional anticancer therapy comprises an immunotherapy. In some embodiments, the additional anticancer therapy comprises administration of an antibody or antigen-binding portion thereof that specifically binds LAG3, TIGIT, TIM3, NKG2a, OX40, ICOS, MICA, CD137, KIR, TGFβ, IL-10, IL-8, B7-H4, Fas ligand, CXCR4, mesothelin, CD27, GITR, or any combination thereof.

### Anti-LAG-3 Antibodies

Certain aspects of the present disclosure are directed to a method for treating a subject afflicted with a tumor having a high TMB status comprising administering to the subject immunotherapy, wherein the immunotherapy comprises an anti-LAG-3 antibody or antigen-binding portion thereof. The method can further comprise measuring the TMB status of a biological sample obtained from the subject. Additionally, the disclosure contemplates administering an anti-LAG-3 antibody or antigen-binding portion thereof to a subject identified as suitable for such therapy, e.g., based on measurement of a high TMB.

Anti-LAG-3 antibodies of the instant disclosure bind to human LAG-3. Antibodies that bind to LAG-3 have been disclosed in Int'l Publ. No. WO/2015/042246 and U.S. Publ. Nos. 2014/0093511 and 2011/0150892. An exemplary LAG-3 antibody useful in the present disclosure is 25F7 (described in U.S. Publ. No. 2011/0150892). An additional exemplary LAG-3 antibody useful in the present disclosure is BMS-986016. In one embodiment, an anti-LAG-3 antibody useful for the composition cross-competes with 25F7 or BMS-986016. In another embodiment, an anti-LAG-3 antibody useful for the composition binds to the same epitope as 25F7 or BMS-986016. In other embodiments, an anti-LAG-3 antibody comprises six CDRs of 25F7 or BMS-986016.

### Anti-CD137 Antibodies

Certain aspects of the present disclosure are directed to a method for treating a subject afflicted with a tumor having a high TMB status comprising administering to the subject immunotherapy, wherein the immunotherapy comprises an anti-CD137 antibody or antigen-binding portion thereof. The method can further comprise measuring the TMB status of a biological sample obtained from the subject. Additionally, the disclosure contemplates administering an anti-CD137 antibody or antigen-binding portion thereof to a subject identified as suitable for such therapy, e.g., based on measurement of a high TMB.

Anti-CD137 antibodies specifically bind to and activate CD137-expressing immune cells, stimulating an immune response, in particular a cytotoxic T cell response, against tumor cells. Antibodies that bind to CD137 have been disclosed in U.S. Publ. No. 2005/0095244 and U.S. Pat. Nos. 7,288,638, 6,887,673, 7,214,493, 6,303,121, 6,569,997, 6,905,685, 6,355,476, 6,362,325, 6,974,863, and 6,210,669.

In some embodiments, the anti-CD137 antibody is urelumab (BMS-663513), described in U.S. Pat. No. 7,288,638 (20H4.9-IgG4 [10C7 or BMS-663513]). In some embodiments, the anti-CD137 antibody is BMS-663031 (20H4.9-IgG1), described in U.S. Pat. No. 7,288,638. In some embodiments, the anti-CD137 antibody is 4E9 or BMS-554271, described in U.S. Pat. No. 6,887,673. In some embodiments, the anti-CD137 antibody is an antibody disclosed in U.S. Pat. Nos. 7,214,493; 6,303,121; 6,569,997; 6,905,685; or 6,355,476. In some embodiments, the anti-CD137 antibody is 1D8 or BMS-469492; 3H3 or BMS-469497; or 3E1, described in U.S. Pat. No. 6,362,325. In some embodiments, the anti-CD137 antibody is an antibody disclosed in issued U.S. Pat. No. 6,974,863 (such as 53A2). In some embodiments, the anti-CD137 antibody is an antibody disclosed in issued U.S. Pat. No. 6,210,669 (such as 1D8, 3B8, or 3E1). In some embodiments, the antibody is Pfizer's PF-05082566 (PF-2566). In other embodiments, an anti-CD137 antibody useful for the disclosure cross-competes with the anti-CD137 antibodies disclosed herein. In some embodiments, an anti-CD137 antibody binds to the same epitope as the anti-CD137 antibody disclosed herein. In other embodiments, an anti-CD137 antibody useful in the disclosure comprises six CDRs of the anti-CD137 antibodies disclosed herein.

### Anti-KIR Antibodies

Certain aspects of the present disclosure are directed to a method for treating a subject afflicted with a tumor having a high TMB status comprising administering to the subject immunotherapy, wherein the immunotherapy comprises an anti-KIR antibody or antigen-binding portion thereof. The method can further comprise measuring the TMB status of a biological sample obtained from the subject. Additionally, the disclosure contemplates administering an anti-KIR antibody or antigen-binding portion thereof to a subject identified as suitable for such therapy, e.g., based on measurement of a high TMB.

Antibodies that bind specifically to KIR block the interaction between Killer-cell immunoglobulin-like receptors (KIR) on NK cells with their ligands. Blocking these receptors facilitates activation of NK cells and, potentially, destruction of tumor cells by the latter. Examples of anti-KTR antibodies have been disclosed in Int'l Publ. Nos. WO/2014/055648, WO 2005/003168, WO 2005/009465, WO 2006/072625, WO 2006/072626, WO 2007/042573, WO 2008/084106, WO 2010/065939, WO 2012/071411 and WO/2012/160448.

One anti-KIR antibody useful in the present disclosure is lirilumab (also referred to as BMS-986015, IPH2102, or the S241P variant of 1-7F9), first described in Int'l Publ. No. WO 2008/084106. An additional anti-KIR antibody useful in the present disclosure is 1-7F9 (also referred to as IPH2101), described in Int'l Publ. No. WO 2006/003179. In one embodiment, an anti-KIR antibody for the present composition cross competes for binding to KIR with lirilumab or 1-7F9. In another embodiment, an anti-KIR antibody binds to the same epitope as lirilumab or 1-7F9. In other embodiments, an anti-KIR antibody comprises six CDRs of lirilumab or 1-7F9.

### Anti-GITR Antibodies

Certain aspects of the present disclosure are directed to a method for treating a subject afflicted with a tumor having a high TMB status comprising administering to the subject immunotherapy, wherein the immunotherapy comprises an anti-GITR antibody or antigen-binding portion thereof. The method can further comprise measuring the TMB status of a biological sample obtained from the subject. Additionally, the disclosure contemplates administering an anti-GITR antibody or antigen-binding portion thereof to a subject identified as suitable for such therapy, e.g., based on measurement of a high TMB.

Anti-GITR antibodies can be any anti-GITR antibody that binds specifically to human GITR target and activates the glucocorticoid-induced tumor necrosis factor receptor (GITR). GITR is a member of the TNF receptor superfamily that is expressed on the surface of multiple types of immune cells, including regulatory T cells, effector T cells, B cells, natural killer (NK) cells, and activated dendritic cells (“anti-GITR agonist antibodies”). Specifically, GITR activation increases the proliferation and function of effector T cells, as well as abrogating the suppression induced by activated T regulatory cells. In addition, GITR stimulation promotes anti-tumor immunity by increasing the activity of other immune cells such as NK cells, antigen presenting cells, and B cells. Examples of anti-GITR antibodies have been disclosed in Int'l Publ. Nos. WO/2015/031667, WO2015/184,099, WO2015/026,684, WO11/028683 and WO/2006/105021, U.S. Pat. Nos. 7,812,135 and 8,388,967 and U.S. Publ. Nos. 2009/0136494, 2014/0220002, 2013/0183321 and 2014/0348841.

In one embodiment, an anti-GITR antibody useful in the present disclosure is TRX518 (described in, for example, Schaer et al. Curr Opin Immunol. (2012) April; 24(2): 217-224, and WO/2006/105021). In another embodiment, the anti-GITR antibody is selected from MK4166, MK1248, and antibodies described in WO11/028683 and U.S. Pat. No. 8,709,424, and comprising, e.g., a VH chain comprising SEQ ID NO: 104 and a VL chain comprising SEQ ID NO: 105 (wherein the SEQ ID NOs are from WO11/028683 or U.S. Pat. No. 8,709,424). In certain embodiments, an anti-GITR antibody is an anti-GITR antibody that is disclosed in WO2015/031667, e.g., an antibody comprising VH CDRs 1-3 comprising SEQ ID NOs: 31, 71 and 63 of WO2015/031667, respectively, and VL CDRs 1-3 comprising SEQ ID NOs: 5, 14 and 30 of WO2015/031667. In certain embodiments, an anti-GITR antibody is an anti-GITR antibody that is disclosed in WO2015/184099, e.g., antibody Hum231 #1 or Hum231 #2, or the CDRs thereof, or a derivative thereof (e.g., pab1967, pab1975 or pab1979). In certain embodiments, an anti-GITR antibody is an anti-GITR antibody that is disclosed in JP2008278814, WO09/009116, WO2013/039954, US20140072566, US20140072565, US20140065152, or WO2015/026684, or is INBRX-110 (INHIBRx), LKZ-145 (Novartis), or MEDI-1873 (MedImmune). In certain embodiments, an anti-GITR antibody is an anti-GITR antibody that is described in PCT/US2015/033991 (e.g., an antibody comprising the variable regions of 28F3, 18E10 or 19D3). For example, an anti-GITR antibody may be an antibody comprising the following VH and VL chains or the CDRs thereof:

VH:
(SEQ ID NO: 1)
QVQLVESGGGVVQPGRSLRLSCAASGFTFSSYGMHWVRQAPGK
GLEWVAVIWYEGSNKYYADSVKGRFTISRDNSKNTLYLQMNSLR
AEDTAVYYCARGGSMVRGDYYYGMDVWGQGTTVTVS,
and
VL:
(SEQ ID NO: 2)
AIQLTQSPSSLSASVGDRVTITCRASQGISSALAWYQQKPGKAPK
LLIYDASSLESGVPSRFSGSGSGTDFTLTISSLQPEDFATYYCQQ
FNSYPYTFGQGTKLEIK;
or
VH:
(SEQ ID NO: 3)
QVQLVESGGGVVQPGRSLRLSCAASGFTFSSYGFHWVRQAPGKGL
EWVAVIWYAGSNKFYADSVKGRFTISRDNSKNTLYLQMNSLRAED
TAVYYCARGGQLDYYYYYVMDVWGQGTTVTVSS,
and
VL:
(SEQ ID NO: 4)
DIQMTQSPSSLSASVGDRVTITCRASQGISSWLAWYQQKPEKAPK
SLIYAASSLQSGVPSRFSGSGSGTDFTLTISSLQPEDFATYYCQQ
YNSYPYTFGQGTKLEIK;
or
VH:
(SEQ ID NO: 5)
VQLVESGGGVVQPGRSLRLSCAASGFTFSSYGMHWVRQAPGKGLEW
VAVIWYAGSNKYYADSVKGRFTISRDNSKNTLYLQMNSLRAEDTAV
YYCARGGRIAVAFYYSMDVWGQGTTVTVSS,
and
VL:
(SEQ ID NO: 6)
DIQMTQSPSSLSASVGDRVTITCRASQGISSWLAWYQQKPEKAPKSL
IYAASSLQSGVPSRFSGSGSGTDFTLTISSLQPEDFATYYCQQYNSY
PYTFGQGTKLEIK.

In certain embodiments, an antibody comprising a pair of the above VH and VL light chains, or their CDRs, comprises a heavy chain constant region of an IgG1 isotype, either wild type or mutated, e.g., to be effectorless. In one embodiment, an anti-GITR antibody comprises the following heavy and light chains amino acid sequences:

heavy chain:
(SEQ ID NO: 7)
QVQLVESGGGVVQPGRSLRLSCAASGFTFSSYGMHWVRQAPGKGLE
WVAVIWYEGSNKYYADSVKGRFTISRDNSKNTLYLQMNSLRAEDTA
VYYCARGGSMVRGDYYYGMDVWGQGTTVTVSSASTKGPSVFPLAPC
SRSTSESTAALGCLVKDYFPEPVTVSWNSGALTSGVHTFPAVLQSS
GLYSLSSVVTVPSSNFGTQTYTCNVDHKPSNTKVDKTVERKCCVEC
PPCPAPPVAGPSVFLFPPKPKDTLMISRTPEVTCVVVDVSHEDPEV
QFNWYVDGVEVHNAKTKPREEQFNSTFRVVSVLTVVHQDWLNGK
EYKCKVSNKGLPAPIEKTISKTKGQPREPQVYTLPPSREEMTKNQV
SLTCLVKGFYPSDIAVEWESNGQPENNYKTTPPMLDSDGSFFLYSK
LTVDKSRWQQGNVFSCSVMHEALHNHYTQKSLSLSPG,
and
light chain:
(SEQ ID NO: 8)
AIQLTQSPSSLSASVGDRVTITCRASQGISSALAWYQQKPGKAPKLL
IYDASSLESGVPSRFSGSGSGTDFTLTISSLQPEDFATYYCQQFNSY
PYTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC,
or
heavy chain:
(SEQ ID NO: 9)
qvqlvesgggvvqpgrslrlscaasgftfssygmhwvrqapgkglew
vaviwyegsnkyyadsvkgrftisrdnskntlylqmnslraedtavy
ycarggsmvrgdyyygmdvwgqgttvtvssastkgpsvfplapssks
tsggtaalgclvkdyfpepvtvswnsgaltsgvhtfpavlqssglys
lssvvtvpssslgtqtyicnvnhkpsntkvdkrvepkscdkthtcpp
cpapeaegapsvflfppkpkdtlmisrtpevtovvvdvshedpevkf
nwyvdgvevhnaktkpreeqynstyrvvsvltvlhqdwlngkeykck
vsnkalpssiektiskakgqprepqvytlppsreemtknqvsltclv
kgfypsdiavewesngqpennykttppvldsdgsfflyskltvdksr
wqqgnvfscsvmhealhnhytqkslslspg,
and
light chain:
(SEQ ID NO: 10)
AIQLTQSPSSLSASVGDRVTITCRASQGISSALAWYQQKPGKAPKLLI
YDASSLESGVPSRFSGSGSGTDFTLTISSLQPEDFATYYCQQFNSYPY
TFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC.

In certain embodiments, the anti-GITR antibody cross-competes with an anti-GITR antibody described herein, e.g., TRX518, MK4166 or an antibody comprising a VH domain and a VL domain amino acid sequence described herein. In some embodiments, the anti-GITR antibody binds the same epitope as that of an anti-GITR antibody described herein, e.g., TRX518, MK4166 or an antibody comprising a VH domain and a VL domain amino acid sequence described herein. In certain embodiments, the anti-GITR antibody comprises the six CDRs of TRX518, MK4166 or those of an antibody comprising a VH domain and a VL domain amino acid sequence described herein.

### Additional Antibodies

In some embodiments, the immunotherapy comprises an anti-TGFβ antibody. In certain embodiments, the anti-TGFβ antibody is an anti-TGFβ antibody disclosed in Int'l Publ. No. WO/2009/073533.

In some embodiments, the immunotherapy comprises an anti-IL-10 antibody. In certain embodiments, the anti-IL-10 antibody is an anti-IL-10 antibody disclosed in Int'l Publ. No. WO/2009/073533.

In some other embodiments, the immunotherapy comprises an anti-B7-H4 antibody. In certain embodiments, the anti-B7-H4 antibody is an anti-B7-H4 antibody disclosed in Int'l Publ. No. WO/2009/073533.

In certain embodiments, the immunotherapy comprises an anti-Fas ligand antibody. In certain embodiments, the anti-Fas ligand antibody is an anti-Fas ligand antibody disclosed in Int'l Publ. No. WO/2009/073533.

In some embodiments, the immunotherapy comprises an anti-CXCR4 antibody. In certain embodiments, the anti-CXCR4 antibody is an anti-CXCR4 antibody disclosed in U.S. Publ. No. 2014/0322208 (e.g., Ulocuplumab (BMS-936564)).

In some embodiments is the immunotherapy comprises an anti-mesothelin antibody. In certain embodiments, the anti-mesothelin antibody is an anti-mesothelin antibody disclosed in U.S. Pat. No. 8,399,623.

In some embodiments, the immunotherapy comprises an anti-HER2 antibody. In certain embodiments, the anti-HER2 antibody is Herceptin (U.S. Pat. No. 5,821,337), trastuzumab, or ado-trastuzumab emtansine (Kadcyla, e.g., WO/2001/000244).

In embodiments, the immunotherapy comprises an anti-CD27 antibody. In embodiments, the anti-CD-27 antibody is Varlilumab (also known as “CDX-1127” and “1F5”), which is a human IgG1 antibody that is an agonist for human CD27, as disclosed in, for example, U.S. Pat. No. 9,169,325.

In some embodiments, the immunotherapy comprises an anti-CD73 antibody. In certain embodiments, the anti-CD73 antibody is CD73.4.IgG2C219S.IgG1.1f.

In some embodiments, the immunotherapy comprises an anti-MICA antibody. As used herein, an anti-MICA antibody is an antibody or an antigen binding fragment thereof that specifically binds MHC class I polypeptide-related sequence A. In some embodiments, the anti-MICA antibody binds MICB in addition to MICA. In some embodiments, the anti-MICA antibody inhibits cleavage of membrane bound MICA and release of soluble MICA. In certain embodiments, the anti-MICA antibody is an anti-MICA antibody disclosed in U.S. Publ. No. 2014/004112 A1, U.S. Publ. No. 2016/046716 A1, or U.S. Publ. No. 2017/022275 A1.

In some embodiments, the immunotherapy comprises an anti-TIM3 antibody. As used herein, an anti-TIM3 antibody is an antibody or an antigen binding fragment thereof that specifically binds T-cell immunoglobulin and mucin-domain containing-3 (TIM3), also known as hepatitis A virus cellular receptor 2 (HAVCR2). In some embodiments, the anti-TIM3 antibody is capable of stimulating an immune response, e.g., an antigen-specific T cell response. In some embodiments, the anti-TIM3 antibody binds to soluble or membrane bound human or cyno TIM3. In certain embodiments, the anti-TIM3 antibody is an anti-TIM3 antibody disclosed in International Publication No. WO/2018/013818, which is incorporated by reference herein in its entirety.

In certain embodiments, the additional anticancer therapy is administered concurrently with, after, or concurrently with and after the administration of the anti-PD-1 antibody (or the anti-PD-L1 antibody) and an anti-CTLA-4 antibody. In some embodiments, the additional anticancer therapy is administered concurrently with the administration of the anti-PD-1 antibody (or the anti-PD-L1 antibody) and an anti-CTLA-4 antibody. In some embodiments, the additional anticancer therapy is administered after the administration of the anti-PD-1 antibody (or the anti-PD-L1 antibody) and an anti-CTLA-4 antibody. In some embodiments, the additional anticancer therapy is administered concurrently with and after the administration of the anti-PD-1 antibody (or the anti-PD-L1 antibody) and an anti-CTLA-4 antibody. In other embodiments, the additional anticancer therapy is administered between the anti-PD-1 antibody (or the anti-PD-L1 antibody) and an anti-CTLA-4 antibody. In certain embodiments, the additional anticancer therapy, the anti-PD-1 antibody (or the anti-PD-L1 antibody), and/or an anti-CTLA-4 antibody are combined in a single formulation. In other embodiments, the additional anticancer therapy, the anti-PD-1 antibody (or the anti-PD-L1 antibody), and/or an anti-CTLA-4 antibody are in separate formulations.

### Pharmaceutical Compositions and Dosages

Therapeutic agents of the present disclosure can be constituted in a composition, e.g., a pharmaceutical composition containing an antibody and/or a cytokine and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. Preferably, the carrier for a composition containing an antibody is suitable for intravenous, intramuscular, subcutaneous, parenteral, spinal or epidermal administration (e.g., by injection or infusion), whereas the carrier for a composition containing an antibody and/or a cytokine is suitable for non-parenteral, e.g., oral, administration. In some embodiments, the subcutaneous injection is based on Halozyme Therapeutics' ENHANZE® drug-delivery technology (see U.S. Pat. No. 7,767,429, which is incorporated by reference herein in its entirety). ENHANZE® uses a co-formulation of an antibody with recombinant human hyaluronidase enzyme (rHuPH20), which removes traditional limitations on the volume of biologics and drugs that can be delivered subcutaneously due to the extracellular matrix (see U.S. Pat. No. 7,767,429). A pharmaceutical composition of the disclosure can include one or more pharmaceutically acceptable salts, anti-oxidant, aqueous and non-aqueous carriers, and/or adjuvants such as preservatives, wetting agents, emulsifying agents and dispersing agents. Therefore, in some embodiments, the pharmaceutical composition for the present disclosure can further comprise recombinant human hyaluronidase enzyme, e.g., rHuPH20.

In some embodiments, the anti-PD-1 antibody or the anti-PD-L1 antibody is administered in a fixed dose with the anti-CTLA-4 antibody in a single composition. In some embodiments, the anti-PD-1 antibody is administered in a fixed dose with the anti-CTLA-4 antibody. In some embodiments, the anti-PD-L1 antibody is administered in a fixed dose with the anti-CTLA-4 antibody in a single composition. In some embodiments, the ratio of the anti-PD-1 antibody or the anti-PD-L1 antibody to the anti-CTLA-4 antibody is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg.

Although higher nivolumab monotherapy dosing up to 10 mg/kg every two weeks has been achieved without reaching the maximum tolerated does (MTD), the significant toxicities reported in other trials of checkpoint inhibitors plus anti-angiogenic therapy (see, e.g., Johnson et al., 2013; Rini et al., 2011) support the selection of a nivolumab dose lower than 10 mg/kg.

Treatment is continued as long as clinical benefit is observed or until unacceptable toxicity or disease progression occurs. Nevertheless, in certain embodiments, the dosages of the anti-PD-1 antibody, the anti-PD-L1 antibody, and/or the anti-CTLA-4 antibody administered are significantly lower than the approved dosage, i.e., a subtherapeutic dosage, of the agent. The anti-PD-1 antibody, the anti-PD-L1 antibody, and/or the anti-CTLA-4 antibody can be administered at the dosage that has been shown to produce the highest efficacy as monotherapy in clinical trials, e.g., about 3 mg/kg of nivolumab administered once every three weeks (Topalian et al., 2012a; Topalian et al., 2012), or at a significantly lower dose, i.e., at a subtherapeutic dose.

Dosage and frequency vary depending on the half-life of the antibody in the subject. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present disclosure can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present disclosure can be administered via one or more routes of administration using one or more of a variety of methods well known in the art. As will be appreciated by the skilled artisan, the route and/or mode of administration will vary depending upon the desired results.

### Kits

Also within the scope of the present disclosure are kits comprising (a) an anti-PD-1 antibody or an anti-PD-L1 antibody and (b) an anti-CTLA-4 antibody for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a tumor derived from a NSCLC, the kit comprising: (a) a dosage ranging from 0.1 to 10 mg/kg body weight of an anti-PD-1 antibody or a dosage ranging from 0.1 to 20 mg/kg body weight of an anti-PD-L1 antibody; (b) a dosage ranging from 0.1 to 10 mg/kg body weight of an anti-CTLA-4 antibody; (c) instructions for using (a) the anti-PD-1 antibody or the anti-PD-L1 antibody and (b) the anti-CTLA-4 antibody in the methods disclosed herein. In some embodiments, this disclosure provides a kit for treating a subject afflicted with a tumor derived from a NSCLC, the kit comprising: (a) a dosage ranging from 200 mg to 800 mg of an anti-PD-1 antibody or a dosage ranging from 200 mg to 1800 mg of an anti-PD-L1 antibody; (b) a dosage ranging from 10 mg to 800 mg of an anti-CTLA-4 antibody; (c) instructions for using (a) the anti-PD-1 antibody or the anti-PD-L1 antibody and (b) the anti-CTLA-4 antibody in the methods disclosed herein.

In certain preferred embodiments for treating human patients, the kit comprises an anti-human PD-1 antibody disclosed herein, e.g., nivolumab or pembrolizumab. In certain preferred embodiments for treating human patients, the kit comprises an anti-human PD-L1 antibody disclosed herein, e.g., atezolizumab, durvalumab, or avelumab. In certain preferred embodiments for treating human patients, the kit comprises an anti-human CTLA-4 antibody disclosed herein, e.g., ipilimumab, tremelimumab, MK-1308, or AGEN-1884.

In some embodiments, the kit further comprises a cytokine or a variant thereof. In certain embodiments the kit comprises (a) an anti-PD-1 antibody or an anti-PD-L1 antibody, (b) an anti-CTLA-4 antibody, and (c) a CD122 agonist.

In some embodiments, the kit further includes a comprehensive genomic profiling assay disclosed herein. In some embodiments, the kit includes a FOUNDATIONONE® CDX™ genomic profiling assay. In some embodiments, the kit further includes instructions to administer (a) the anti-PD-1 antibody or the anti-PD-L1 antibody and (b) the anti-CTLA-4 antibody to a subject identified as having a high TMB status, e.g., a TMB status of at least about 10 mutations/Mb of genome sequenced, according to the methods disclosed herein. In other embodiments, the kit further includes instructions to administer (a) the anti-PD-1 antibody or the anti-PD-L1 antibody, (b) the anti-CTLA-4 antibody, and (c) the cytokine, e.g., the CD122 agonist, to a subject identified as having a high TMB status, e.g., a TMB status of at least about 10 mutations/Mb of genome sequenced, according to the methods disclosed herein.

All of the references cited above, as well as all references cited herein, are incorporated herein by reference in their entireties.

The following examples are offered by way of illustration and not by way of limitation.

### EXAMPLES

### Example 1: Nivolumab Plus Ipilimumab in High Tumor Mutational Burden in Non-Small Cell Lung Cancer

Nivolumab+ipilimumab demonstrated promising efficacy in a phase 1 NSCLC study, and tumor mutational burden (TMB) has emerged as a potential biomarker of benefit. This trial is an open-label, multi-part phase 3 study of first-line nivolumab and nivolumab-based combinations in biomarker-selected NSCLC populations. We report results from part 1 on the co-primary endpoint of progression-free survival (PFS) with nivolumab+ipilimumab versus chemotherapy in patients with high TMB (≥10 mutations/Mb). The study continues for the co-primary endpoint of overall survival in PD-L1-selected patients.

Patients had chemotherapy-naive, stage IV or recurrent NSCLC. Those with ≥1% tumor PD-L1 expression were randomized 1:1:1 to nivolumab+ipilimumab, nivolumab, or chemotherapy; those with <1% tumor PD-L1 expression were randomized 1:1:1 to nivolumab+ipilimumab, nivolumab+chemotherapy, or chemotherapy. TMB was determined using FOUNDATIONONE® CDX™

PFS in patients with high TMB (≥10 mutations/Mb) was significantly longer with nivolumab+ipilimumab versus chemotherapy (HR, 0.58; 97.5% CI, 0.41-0.81; P=0.0002); 1-year PFS rates were 43% and 13%, and median PFS (95% CI) was 7.2 (5.5-13.2) and 5.5 (4.4-5.8) months, respectively. Objective response rates were 45.3% and 26.9%, respectively. Benefit of nivolumab+ipilimumab versus chemotherapy was broadly consistent within subgroups, including those with ≥1% and <1% PD-L1 expression. Grade 3-4 treatment-related adverse events rates were 31% and 36%, respectively.

PFS improved significantly with first-line nivolumab+ipilimumab versus chemotherapy in NSCLC with TMB≥10 mutations/Mb, irrespective of PD-L1 expression. The results validate the benefit of nivolumab+ipilimumab in NSCLC and the role of TMB as a biomarker for patient selection.

### Selection of Patients

Fresh or archival tumor-biopsy specimens obtained within 6 months before enrollment (and without the patient receiving any intervening systemic anti-cancer therapy) were tested for PD-L1 by a centralized laboratory with the use of the anti-PD-L1 antibody (28-8 antibody). Hanna, N., et al. J Oncol Pract 13:832-7 (2017).

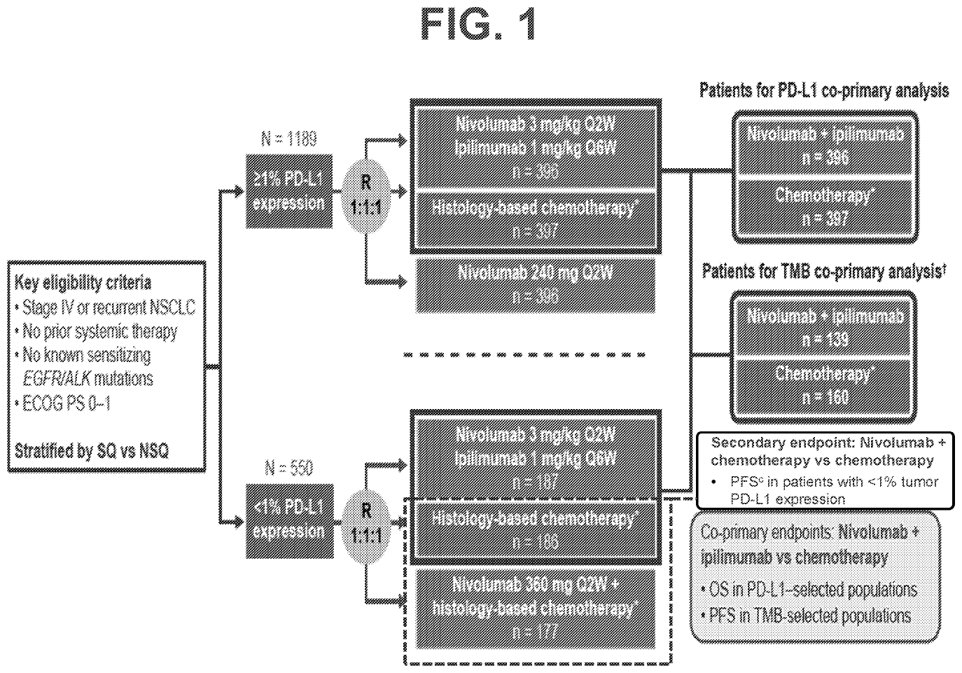

Adult patients with PD-L1-histologically confirmed squamous or nonsquamous stage IV/recurrent NSCLC and Eastern Cooperative Oncology Group (ECOG) performance status (Oken M. M., et al. Am J Clin Oncol 5:649-55 (1982)) of 0 or 1 who had received no prior systemic anticancer therapy as primary therapy for advanced or metastatic disease were eligible for the study. See FIG. 1. All patients underwent imaging to screen for brain metastases. Patients with known EGFR mutations or ALK translocations sensitive to targeted therapy, an autoimmune disease, or untreated central nervous system metastases were excluded. Patients with central nervous system metastases were eligible if they were adequately treated and had neurologically returned to baseline for ≥2 weeks before randomization.

As additional inclusion and exclusion criteria, prior adjuvant or neoadjuvant chemotherapy or prior definitive chemoradiation for locally advanced disease was allowed up to 6 months before enrollment. Prior palliative radiotherapy to non-central nervous system lesions must have been completed ≥2 weeks before randomization. Patients had to be off glucocorticoids or on stable or decreasing doses of ≤10 mg daily prednisone (or equivalent) for ≥2 weeks before randomization.

### Study Design and Treatment

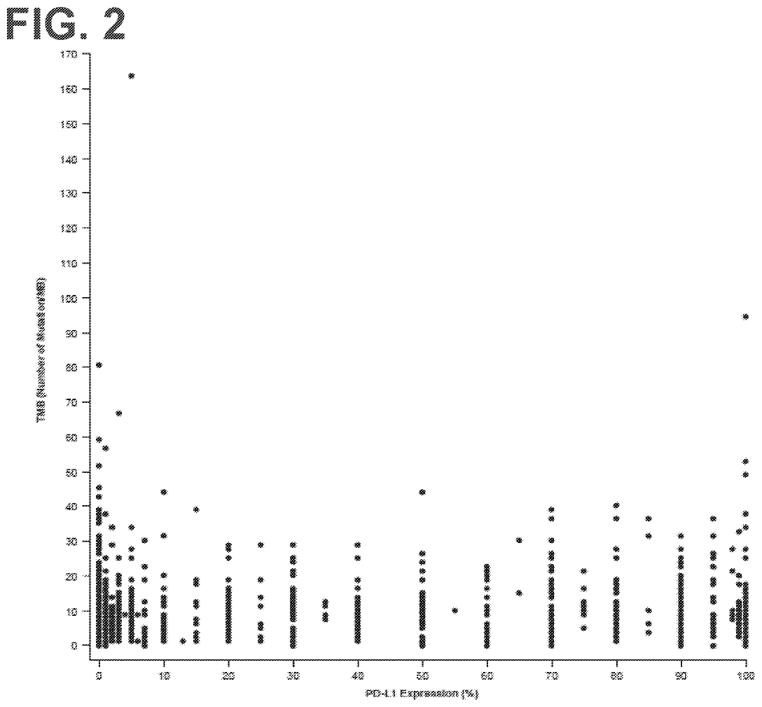

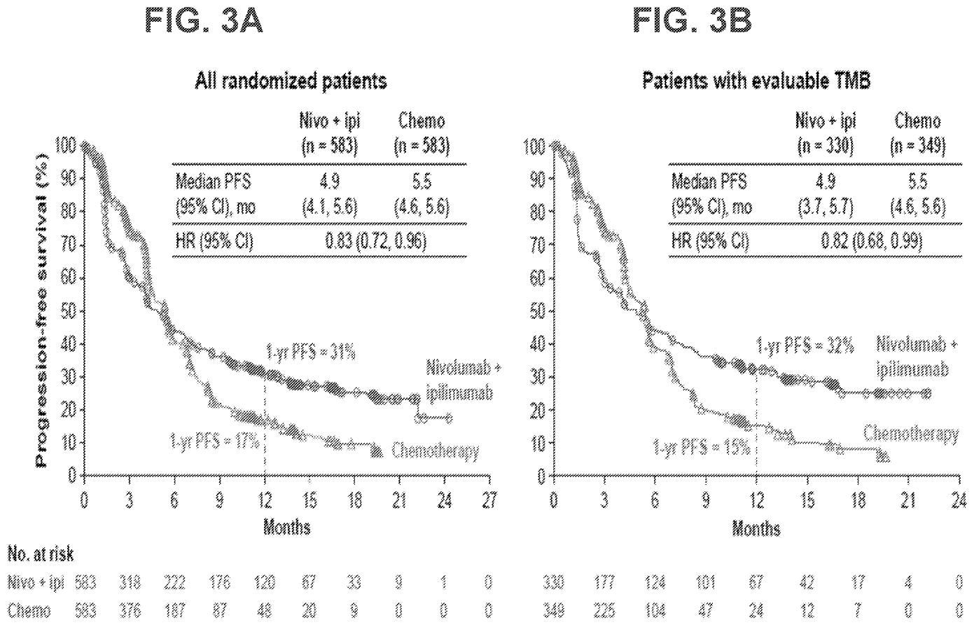

The instant study was a multi-part phase 3 trial designed to evaluate different nivolumab-based regimens vs. chemotherapy in distinct patient populations. For a period of 16 months, patients with ≥1% and <1% tumor PD-L1 expression were enrolled contemporaneously at the same centers (FIG. 2) Patients with ≥1% PD-L1 expression were randomized (1:1:1), stratified by tumor histology (squamous versus nonsquamous NSCLC), to (i) nivolumab 3 mg/kg every 2 weeks plus ipilimumab 1 mg/kg every 6 weeks, (ii) histology-based platinum-doublet chemotherapy every 3 weeks for up to 4 cycles, or (iii) nivolumab 240 mg every 2 weeks. Patients with <1% PD-L1 expression were randomized (1:1:1), stratified by tumor histology, to (i) nivolumab 3 mg/kg every 2 weeks plus ipilimumab 1 mg/kg every 6 weeks, (ii) histology-based platinum-doublet chemotherapy every 3 weeks for up to 4 cycles, or (iii) nivolumab 360 mg plus histology-based platinum-doublet chemotherapy every 3 weeks for up to 4 cycles. Patients with nonsquamous NSCLC with stable disease or response after 4 cycles of chemotherapy or chemotherapy with nivolumab could continue with maintenance pemetrexed or pemetrexed plus nivolumab. All treatments continued until disease progression, unacceptable toxicity, or completion per protocol (up to 2 years for immunotherapy). Crossover between treatment arms within the study was not permitted.

Of 2877 patients enrolled in part 1 of the trial, 1739 underwent randomization. Of the 1138 patients who were not randomized, 909 patients no longer met the study criteria (common reasons included EGFR/ALK mutations identified, decline in ECOG PS, untreated brain metastases, and non-evaluable PD-L1 expression), 88 patients withdrew consent, 40 patients died, 33 patients had adverse events (unrelated to study drug), 6 patients were lost to follow-up, and 62 patients were excluded for other reasons.

As shown in Tables 16 and 17, the baseline characteristics in all randomized and TMB-evaluable patients were similar and balanced between treatment arms.

TABLE 16
Baseline Characteristics of All Randomized Patients.

| | All randomized patients | | | |
|---|---|---|---|---|
| | Nivolumab + | | Chemo- | |
| | Ipilimumab | Nivolumab | therapy | Total |
| | (n = 583) | (n = 396) | (n = 583) | (N = 1739) |
| Median age, years | 64 | 64 | 64 | 64 |
| Female, % | 33 | 31 | 34 | 32 |
| ECOG PS, % | | | | |
| 0 | 35 | 36 | 33 | 34 |
| 1 | 65 | 64 | 66 | 65 |
| ≥2 | >1 | 0 | 1 | <1 |
| Not reported | 0 | <1 | <1 | <1 |
| Smoking status, % | | | | |
| Current/former | 85 | 86 | 86 | 85 |
| smoker | | | | |
| Never smoker | 14 | 13 | 13 | 13 |
| Unknown | 1 | 1 | 1 | 1 |
| Histology, % | | | | |
| Squamous | 28 | 30 | 28 | 28 |
| Non-squamous | 72 | 70 | 72 | 72 |
| PD-L1 expression, | | | | |
| % | | | | |
| <1% | 32 | 0 | 32 | 32 |
| ≥1% | 68 | 100 | 68 | 68 |

ECOG PS = Eastern Cooperative Oncology Group performance status;
PD-L1 = programmed death ligand 1.

TABLE 17
Baseline Characteristics of All TMB-evaluable Patients.

| | TMB evaluable patients | | | |
|---|---|---|---|---|
| | Nivolumab + | | Chemo- | |
| | Ipilimumab | Nivolumab | therapy | Total |
| | (n = 330) | (n = 228) | (n = 349) | (N = 1004) |
| Median age, years | 64 | 64 | 64 | 64 |
| Female, % | 34 | 31 | 36 | 33 |
| ECOG PS, % | | | | |
| 0 | 33 | 32 | 34 | 33 |
| 1 | 67 | 67 | 65 | 67 |
| ≥2 | <1 | 0 | 1 | <1 |
| Not reported | 0 | <1 | <1 | <1 |
| Smoking status, % | | | | |
| Current/former | 86 | 86 | 87 | 87 |
| smoker | | | | |
| Never smoker | 12 | 12 | 11 | 12 |
| Unknown | 2 | 1 | 1 | 1 |
| Histology, % | | | | |
| Squamous | 28 | 29 | 32 | 29 |
| Non-squamous | 72 | 71 | 68 | 71 |
| PD-L1 expression, | | | | |
| % | | | | |
| <1% | 27 | 0 | 31 | 29 |
| ≥1% | 73 | 100 | 69 | 71 |

ECOG PS = Eastern Cooperative Oncology Group performance status

### Tumor Mutation Burden Analysis

TMB was assessed in archival or fresh formalin-fixed, paraffin-embedded tumor samples using the validated assay FOUNDATIONONE® CDX™, which employs next generation sequencing to detect substitutions, insertions and deletion (indels), and copy number alterations in 324 genes and select gene rearrangements. Ettinger, D. S., et al. J Natl Compr Canc Netw, 15:504-35 (2017). Independent reports have demonstrated concordance between TMB estimated from whole exome sequencing (WES) and TMB estimated from targeted next generation sequencing (NGS). See Szustakowski J., et al. Evaluation of tumor mutation burden as a biomarker for immune checkpoint inhibitor efficacy: A calibration study of whole exome sequencing with FoundationOne®. Presented at the American Association for Cancer Research 2018 Annual Meeting; 2018; Chicago, Illinois; Zehir A, et al. Nat Med 2017; 23:703-713; Rizvi H., et al., J Clin Oncol 2018; 36:633-41. TMB was calculated according to previously defined methods. Reck, M., et al., N Engl J Med, 375:1823-33 (2016). Briefly, TMB was defined as the number of somatic, coding, base substitution and short indels per megabase of genome examined. All base substitutions and indels in the coding region of targeted genes, including synonymous mutations, were filtered for both oncogenic driver events according to COSMIC and germline status according to dbSNP and ExAC databases, in addition to a private database of rare germline events compiled in the Foundation Medicine clinical cohort. Additional filtering based upon a computational assessment of germline status using the SGZ (somatic-germline-zygosity) tool was also performed. Aguiar, P. N., et al., ESMO Open, 2:e000200 (2017).

As shown in Table 18, of all randomized patients (N=1739), 1649 (95%) had tumor samples for TMB assessment, and 1004 (58%) had valid TMB data for TMB-based efficacy analyses.

TABLE 18
Sample Size Throughout TMB Determination
Patients, n (%)

| | |
|---|---|
| Randomized^{a} | 1739 (100) |
| Samples available | 1649 (95) |
| TMB-evaluable samples^{b} | 1004 (58) |

^{a}Randomized patients include those from all treatment arms in Part 1 (nivolumab + ipilimumab, nivolumab, chemotherapy, and nivolumab + chemotherapy arms)
^{b}A pre-analytical quality control check was performed on all samples to flag inaccuracies comprised of but not limited to incorrect requisitions, receipt of insufficient sample, and duplicate samples. The FOUNDATIONONE ® CDX ™ assay employs comprehensive quality control criteria, including the following critical characteristics: tumor purity, DNA sample size, tissue sample size, library construction size, and hybrid capture yields.

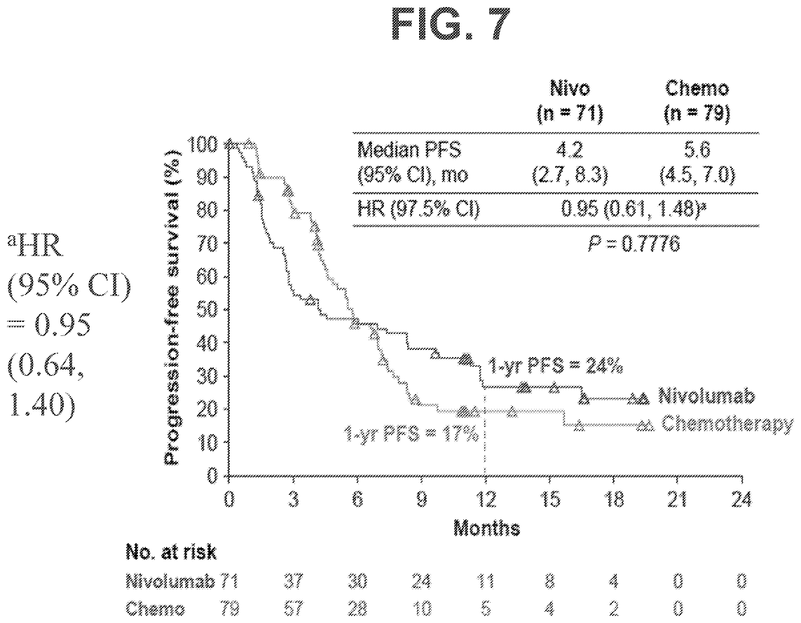

Of all TMB-evaluable patients across all treatment arms, 444 (44%) had TMB≥10 mutations/Mb, including 139 patients randomized to nivolumab plus ipilimumab and 160 patients randomized to chemotherapy. As shown in Table 19, baseline characteristics between the two treatment groups were well balanced, including distribution of PD-L1 expression. In the TMB-evaluable population, there was no correlation between TMB and PD-L1 expression. FIGS. 7A and 7B.

TABLE 19
Baseline Characteristics of Patients with TMB ≥10 mutations/Mb.

| | Nivolumab | |
|---|---|---|
| | plus | |
| | Ipilimumab | Chemotherapy |
| Characteristic | (n = 139) | (n = 160) |
| Age, years | | |
| Median | 64 | 64 |
| Range | 41-87 | 29-80 |
| Age category, n (%) | | |
| <65 years | 73 (53) | 83 (52) |
| ≥65 to <75 years | 53 (38) | 63 (39) |
| ≥75 years | 13 (9) | 14 (9) |
| Sex, n (%) | | |
| Male | 98 (71) | 106 (66) |
| Female | 41 (29) | 54 (34) |
| Region, n (%) | | |
| North America | 14 (10) | 16 (10) |
| Europe | 77 (55) | 87 (54) |
| Asian | 21 (15) | 32 (20) |
| Rest of World | 27 (19) | 25 (16) |
| ECOG performance-status score, | | |
| n (%) | | |
| 0 | 56 (40) | 49 (31) |
| 1 | 82 (59) | 110 (69) |
| ≥2 | 1 (1) | 1 (1) |
| Smoking status, n (%) | | |
| Current/Former Smoker | 130 (94) | 146 (91) |
| Never smoker | 7 (5) | 11 (7) |
| Unknown | 2 (1) | 3 (2) |
| Tumor histology, n (%) | | |
| Squamous cell carcinoma | 45 (32) | 55 (34) |
| Nonsquamous cell carcinoma | 94 (68) | 105 (66) |
| PD-L1 expression level, n (%) | | |
| <1% | 38 (27) | 48 (30) |
| ≥1% | 101 (73) | 112 (70) |

At a minimum follow-up of 11.2 months, 17.7% and 5.6% of patients treated with nivolumab plus ipilimumab and chemotherapy, respectively remained on treatment. See Table 20.

TABLE 20
End-of-Treatment Summary.

| | | | TMB ≥10 | |
|---|---|---|---|---|
| | All Treated Patients | | mutations/Mb | |
| | Nivolumab + | Chemo- | Nivolumab + | Chemo- |
| | Ipilimumab | therapy | Ipilimumab | therapy |
| | n = 576 | n = 570 | n = 135 | n = 159 |
| Patients continuing in the treatment | 102 (17.7) | 32 (5.6) | 33 (24.2) | 5 (3.1) |
| period, n (%) | | | | |
| Patients not continuing in the | 474 (82.3) | 538 (94.4) | 102 (75.6) | 154 (96.9) |
| treatment period, n (%) | | | | |
| Reason for not continuing in the | | | | |
| treatment period, n (%) | | | | |
| Disease progression | 285 (49.5) | 279 (48.9) | 51 (37.8) | 75 (47.2) |
| Study drug toxicity | 108 (18.8) | 51 (8.9) | 35 (25.9) | 14 (8.8) |
| Completed required treatment | 2 (0.3) | 126 (22.1) | 0 | 42 (26.4) |
| Death | 6 (1.0) | 2 (0.4) | 1 (0.7) | 0 |
| Adverse event unrelated to study drug | 39 (6.8) | 35 (6.1) | 7 (5.2) | 9 (5.7) |
| Patient request to discontinue | 9 (1.6) | 19 (3.3) | 3 (2.2) | 8 (5.0) |
| Patient withdrew consent | 8 (1.4) | 6 (1.1) | 1 (0.7) | 1 (0.6) |
| Lost to follow-up | 1 (0.2) | 1 (0.2) | 0 | 0 |
| Maximum clinical benefit | 3 (0.5) | 0 | 1 (0.7) | 0 |
| Lack of compliance | 1 (0.2) | 2 (0.4) | 0 | 1 (0.6) |
| Patient no longer meets study criteria | 1 (0.2) | 1 (0.2) | 0 | 0 |
| Other | 11 (1.9) | 10 (1.8) | 3 (2.2) | 2 (1.3) |
| Not reported | 0 | 6 (1.1) | 0 | 2 (1.3) |

Of patients assigned to chemotherapy, 28.100 received subsequent immunotherapy. See Table 21.

TABLE 21
Subsequent Systemic Therapies in Patients With TMB
≥10 mutations/Mb.^{a}

| | Nivolumab + | |
|---|---|---|
| | Ipilimumab | Chemotherapy |
| Patients, n (%) | n = 139 | n = 160 |
| Any subsequent systemic therapy | 23 (16.5) | 69 (43.1) |
| Immunotherapy | 3 (2.2) | 45 (28.1) |
| Anti-PD-1 | 3 (2.2) | 42 (26.3) |
| Nivolumab | 3 (2.2) | 36 (22.5) |
| Pembrolizumab | 0 | 6 (3.8) |
| Anti-PD-L1 (atezolizumab) | 0 | 1 (0.6) |
| Anti-CLTA-4 (ipilimumab) | 0 | 5 (3.1)^{b} |
| Other immunotherapy | 0 | 2 (1.3) |
| Targeted therapy | 2 (1.4) | 3 (1.9) |
| Chemotherapy | 22 (15.8) | 33 (20.6) |

^{a}At the time of database lock, 24% of patients treated with nivolumab + ipilimumab and 3% of those treated with chemotherapy were still on treatment.
^{b}All 5 patients received ipilimumab in combination with nivolumab.

The median duration of therapy was 4.2 months (range, 0.03 to 24.0+) with nivolumab plus ipilimumab and 2.6 months (range, 0.03 to 22.1+) with chemotherapy. The median number of doses of nivolumab (every 2 weeks) and ipilimumab (every 6 weeks) received as combination therapy was 9 (range, 1 to 53) and 3 (range, 1 to 18), respectively.

Among patients with high TMB (≥10 mutations/Mb), 24.2% treated with nivolumab plus ipilimumab and 3.1% treated with chemotherapy were continuing treatment at the time of database lock; the most common reason for discontinuing treatment was disease progression (37.8% and 47.2%, respectively), study drug toxicity (25.9% and 8.8%, respectively), and completion of required treatment among patients in the chemotherapy group (26.4% vs. 0% for patients treated with nivolumab plus ipilimumab)

### Endpoints and Assessments

Part 1 of this study had two co-primary endpoints. One co-primary endpoint was progression-free survival (PFS), which was assessed by blinded independent central review, with nivolumab plus ipilimumab vs. chemotherapy in a TMB-selected patient population. Based on previous findings (Ramalingam S S, et al. Tumor mutation burden (TMB) as a biomarker for clinical benefit from dual immune checkpoint blockade with nivolumab (nivo)+ipilimumab (ipi) in first-line (1L) non-small cell lung cancer (NSCLC): identification of TMB cutoff from CheckMate 568. Presented at the American Association for Cancer Research 2018 Annual Meeting; 2018; Chicago, Illinois.), a predefined TMB cutoff of ≥10 mutations/Mb was selected for preplanned analysis of the co-primary endpoint. The second co-primary endpoint was overall survival (OS) with nivolumab plus ipilimumab vs. chemotherapy in a PD-L1-selected patient population.

As shown in Table 22, secondary endpoints in TMB-selected patient populations included PFS with nivolumab vs. chemotherapy in patients with TMB≥13 mutations/Mb and ≥1% PD-L1 expression and OS with nivolumab plus ipilimumab vs. platinum-doublet chemotherapy in patients with TMB≥10 mutations/Mb.

TABLE 22
Hierarchical Hypothesis Testing in TMB-Selected Patients.

| Hierarchy | Endpoint | Population | Comparison |
|---|---|---|---|
| 1 | Primary endpoint: | TMB ≥10 mutations/Mb | Nivolumab + Ipilimumab |
| | PFS | | vs |
| | Alpha = 0.25 | | Chemotherapy |
| 2 | Secondary endpoint: | TMB ≥13 mutations/Mb | Nivolumab |
| | PFS | and | vs |
| | | ≥1% tumor PD-L1 expression | Chemotherapy |
| 3 | Secondary endpoint: | TMB ≥10 mutations/Mb | Nivolumab + Ipilimumab |
| | OS | | vs |
| | | | Chemotherapy |
| 4 | Secondary endpoint: | TMB ≥13 mutations/Mb | Nivolumab |
| | OS | and | vs |
| | | ≥1% tumor PD-L1 expression | Chemotherapy |

Exploratory endpoints: ORR, PFS for all arms, safety

PFS = progression-free survival;
ORR = objective response rate;
OS = overall survival

The TMB cutoff of ≥13 mutations/Mb for the secondary endpoint of PFS with nivolumab versus chemotherapy was based on analyses from the previous studies, including a bridging study converting whole exome sequencing data to FOUNDATIONONE® CDX™ data. See Carbone et al. N Engl J Med 2017; 376:2415-26; Szustakowski et al. Evaluation of tumor mutation burden as a biomarker for immune checkpoint inhibitor efficacy: A calibration study of whole exome sequencing with FoundationOne®. In: American Association for Cancer Research 2018 Annual Meeting. Chicago, Illinois; 2018. Overall response rates (ORR), duration of response, and safety were exploratory endpoints. Adverse events were graded according to the National Cancer Institute Common Terminology Criteria for Adverse Events, version 4.0. PD-L1 was determined as previously described. See Labeling: PD-L1 IHC 28-8 pharmDx. Dako North America, 2016. (Accessed Oct. 20, 2016, at accessdata.fda.gov/cdrh_docs/pdf15/P150027c.pdf)

TMB, defined as the number of somatic, coding, base substitutions and short insertions and deletions (indels) per megabase of genome examined, was determined using the FOUNDATIONONE® CDX™ assay. See, e.g., FOUNDATIONONE® CDX™. Foundation Medicine, 2018. (Accessed Feb. 8, 2018, at foundationmedicine.com/genomic-testing/foundation-one-cdx.); Chalmers et al., Analysis of 100,000 human cancer genomes reveals the landscape of tumor mutational burden. Genome Med 2017; 9:34; and Sun J X, He Y, Sanford E, et al. The mutation count following application of various filters was divided by the region counted (0.8 Mb) to yield mutations/Mb.

For the co-primary endpoint of PFS with nivolumab plus ipilimumab vs. chemotherapy in patients with TMB≥10 mutations/Mb, it was estimated that a sample size of at least 265 patients with approximately 221 events of death or disease progression would provide 80% power to detect a hazard ratio of 0.66 favoring nivolumab plus ipilimumab vs. chemotherapy, with a two-sided type 1 error of 0.025, by means of a two-sided log-rank test. Hazard ratios of PFS with associated two-sided confidence intervals were estimated using an unstratified Cox proportional hazard model, with treatment group as a single covariate. A multivariate analysis was prespecified in patients with TMB≥10 mutations/Mb to assess the influence of known prognostic baseline factors on PFS. Estimates of hazard ratios with corresponding two-sided 97.5% CI were computed for primary and secondary comparisons specified in the hierarchical hypothesis testing in TMB-selected patients (see Table 22, above); for all other estimates two-sided 95% CI were computed that should not be used to infer differences in treatment effects. Survival curves were estimated using Kaplan-Meier methodology.

In conclusion, this study met its co-primary endpoint, and the results may establish two new standards of care in advanced NSCLC. First, all treatment-naïve NSCLC patients should be tested for TMB as the results validate the role of TMB as an important and independent biomarker. Second, this study introduces nivolumab plus ipilimumab as a new first-line treatment option for patients with high TMB≥10 mutations/Mb. These results provide a more personalized approach to treating lung cancer, by offering effective first-line, chemotherapy-sparing combination immunotherapy to patients who are most likely to receive durable benefit, while preserving effective second-line options. The use of TMB as a predictive biomarker for patients with NSCLC provides an example of precision medicine, tailoring treatment to those patients who will most likely benefit from combination immunotherapy.

### All Randomized Patients

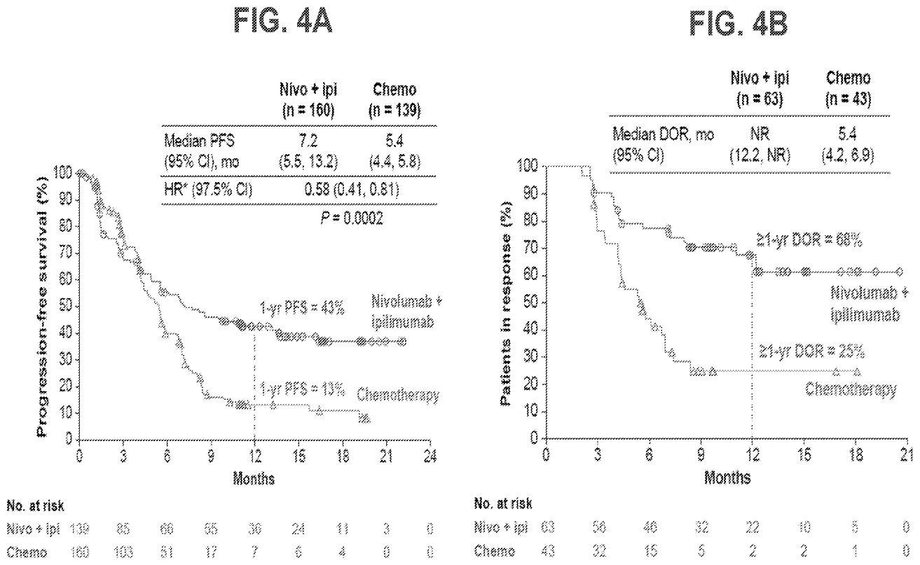

In all randomized patients (irrespective of PD-L1 expression), PFS improved with nivolumab plus ipilimumab vs. chemotherapy (hazard ratio [HR], 0.83; 95%, 0.72 to 0.96), with 1-year PFS rates of 31% versus 17%. The median PFS was 4.9 months (95% CI, 4.1 to 5.6) with nivolumab plus ipilimumab and 5.5 months (95% CI, 4.6 to 5.6) with chemotherapy. Similar benefit with nivolumab plus ipilimumab versus chemotherapy was seen among TMB-evaluable patients (HR, 0.82; 95% CI, 0.68 to 0.99), with 1-year PFS rates of 32% versus 15%; the median PFS was 4.9 months (95% CI, 3.7 to 5.7) and 5.5 months (95% CI, 4.6 to 5.6), respectively. See FIGS. 4A and 4B.

Patients with High TMB (≥10 Mutations/Mb) v. Low TMB

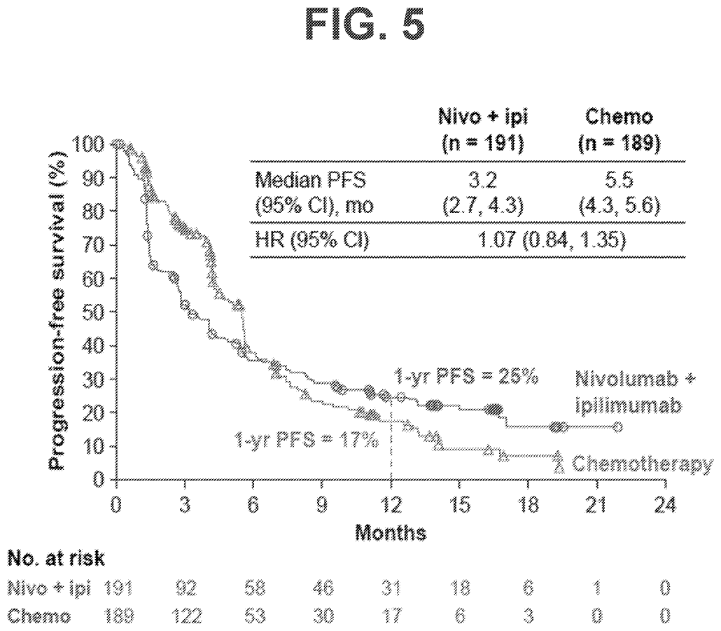

Analysis of the co-primary endpoint in patients with high TMB (≥10 mutations/Mb) showed significant improvement of PFS with nivolumab plus ipilimumab versus chemotherapy (HR, 0.58; 97.5% CI, 0.41 to 0.81; P=0.0002) with the 1-year PFS rates of 43% versus 13% with chemotherapy, and median PFS was 7.2 months (95% CI, 5.5 to 13.2) and 5.5 months (95% CI, 4.4 to 5.8), respectively. FIG. 4A. In a prespecified multivariate analysis of PFS in patients with TMB≥10 mutations/Mb, the treatment effect of nivolumab plus ipilimumab vs chemotherapy adjusted for baseline PD-L1 expression level (≥1%, <1%), gender, tumor histology (squamous, non-squamous) and ECOG PS (0, ≥1) was consistent with the primary PFS analysis (HR, 0.57; 9500 CI, 0.40 to 0.80, multivariate Cox model P=0.0002). In patients with TMB<10 mutations/Mb, no improvement of PFS was observed with nivolumab plus ipilimumab versus chemotherapy (HR, 1.07; 95% CI, 0.84 to 1.35); median PFS was 3.2 months (95% CI, 2.7 to 4.3) with nivolumab plus ipilimumab and 5.5 months (95% CI, 4.3 to 5.6) with chemotherapy. See FIG. 5.

The objective response rate was 4530 with nivolumab plus ipilimumab and 26.90 with chemotherapy (Table 23) Eisenhauer, E. A., et al. Eur J Cancer, 45:228-47 (2009). The percentage of responders with ongoing who still were in response after 1-year was 68 for nivolumab plus ipilimumab and 2500 for chemotherapy (FIG. 4B).

TABLE 23
Tumor Response in Patients with TMB ≥10 mutations/Mb.

| | | |
|---|---|---|
| | Nivolumab plus | |
| | Ipilimumab | Chemotherapy |
| Variable | (n = 139) | (n = 160) |

Objective response†

| | | |
|---|---|---|
| No. of patients | 63 | 43 |
| % of patients (95% CI) | 45.3 (36.9-54.0) | 26.9 (20.2-34.4) |

| | |
|---|---|
| Difference (95% CI) | 18.4 (7.6-28.8) |

Best overall response - no. (%)

| | | |
|---|---|---|
| Complete response | 5 (3.6) | 1 (0.6) |
| Partial response | 58 (41.7) | 42 (26.3) |
| Stable disease | 37 (26.6) | 88 (55.0) |
| Progressive disease | 22 (15.8) | 19 (11.9) |
| Could not be determined | 17 (12.2) | 10 (6.3) |

Time to objective response - mo‡§

| | | |
|---|---|---|
| Median | 2.7 | 1.5 |
| Range | 1.2-9.5 | 1.2-6.9 |

Duration of objective response - mo‡¶

| | | |
|---|---|---|
| Median | NR | 5.4 |
| Range | 2.1-20.5+ | 2.6-18.1+ |

1-year response rate, %

| | | |
|---|---|---|
| Estimate | 68 | 25 |
| 95% confidence interval | 54-78 | 12-40 |

* Data are based on a Jan. 24, 2018, database lock.
†Objective response was assessed according to the Response Evaluation Criteria in Solid Tumors, version 1.1,27 by blinded independent central review. The 95% confidence interval (CI) is based on the Clopper-Pearson method. Unweighted difference in objective response rates between treatment groups was determined by the method of Newcombe.
‡The analysis was performed with data from all the patients who had a response (63 patients in the nivolumab group and 43 in the chemotherapy group).
§The time to response was defined as the time from randomization to the date of first documented complete or partial response.
¶Results were calculated with the use of the Kaplan-Meier method. The duration of response was defined as the time between the date of first response and the date of first documented event of progression, death, or last tumor assessment that was evaluated before subsequent therapy (data-censoring date).
NR denotes not reached.

Selected Subgroups in Patients with High TMB (≥10 Mutations/Mb)

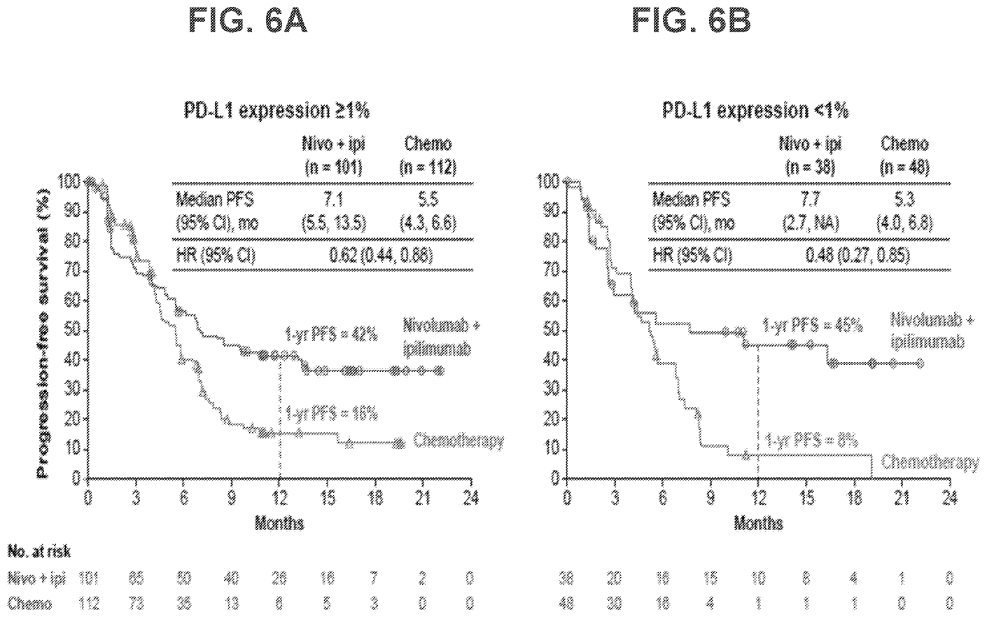

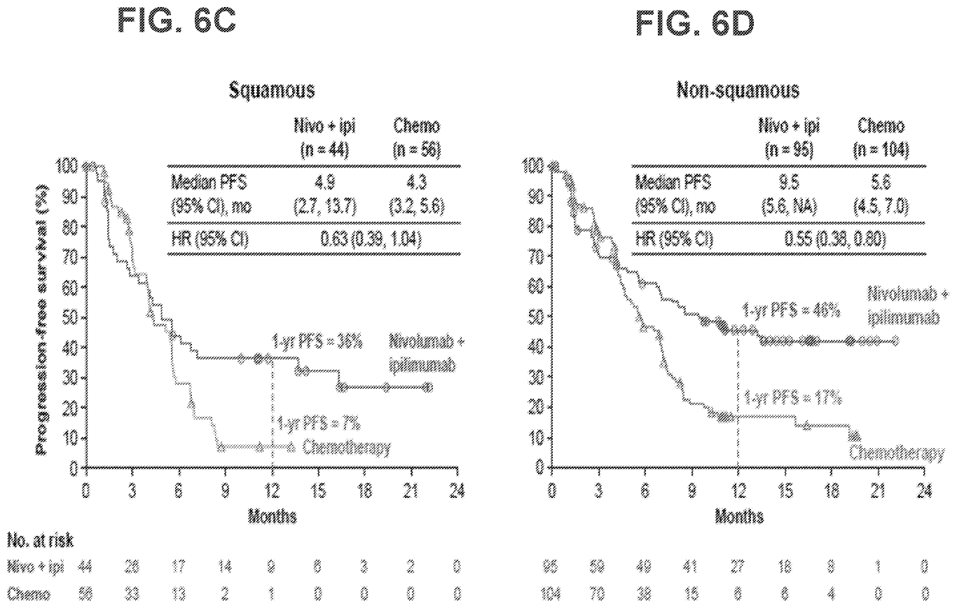

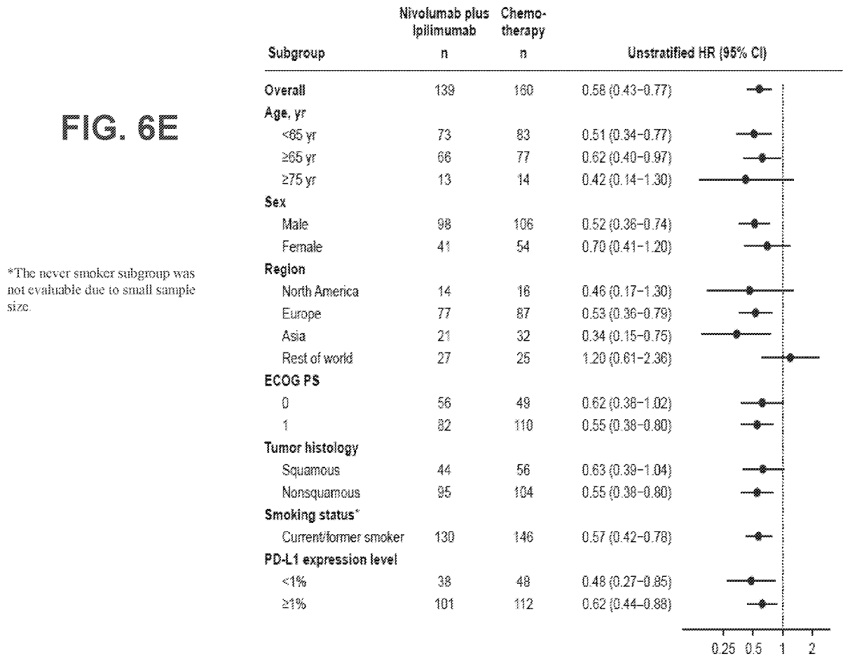

Subgroup analysis by PD-L1 status showed that PFS was improved with nivolumab plus ipilimumab vs. chemotherapy in patients with ≥1% PD-L1 expression and those with <1% PD-L1 expression. FIGS. 6A and 6B Improved PFS with nivolumab plus ipilimumab vs. chemotherapy was seen in patients with both squamous and nonsquamous tumor histology. FIGS. 6C and 6D Across most other subgroups of patients with TMB≥10 mutations/Mb, PFS was improved with nivolumab plus ipilimumab vs. chemotherapy. FIG. 6E.

### Nivolumab Monotherapy

A secondary endpoint of the study was efficacy of nivolumab (n=79) vs. chemotherapy (n=71) among patients with TMB≥13 mutations/Mb and ≥1% PD-L1 expression (patients with <1% PD-L1 expression were not eligible to receive nivolumab); there was no improvement in PFS with nivolumab in this patient group (HR, 0.95; 97.5% CI, 0.61, 1.48; P=0.7776). The median PFS was 4.2 months (95% CI, 2.7 to 8.3) with nivolumab and 5.6 months (95% CI, 4.5 to 7.0) with chemotherapy. FIG. 7.

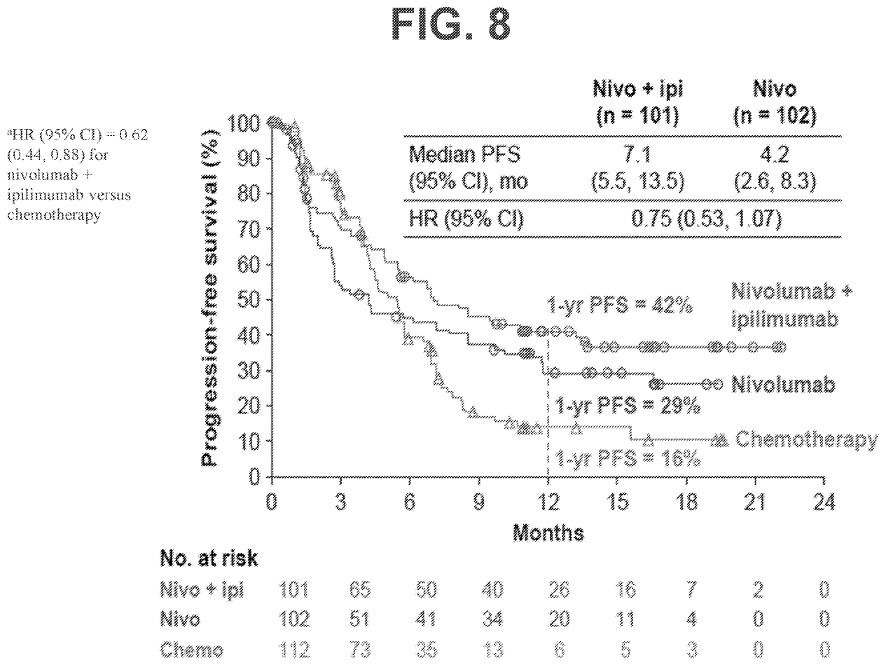

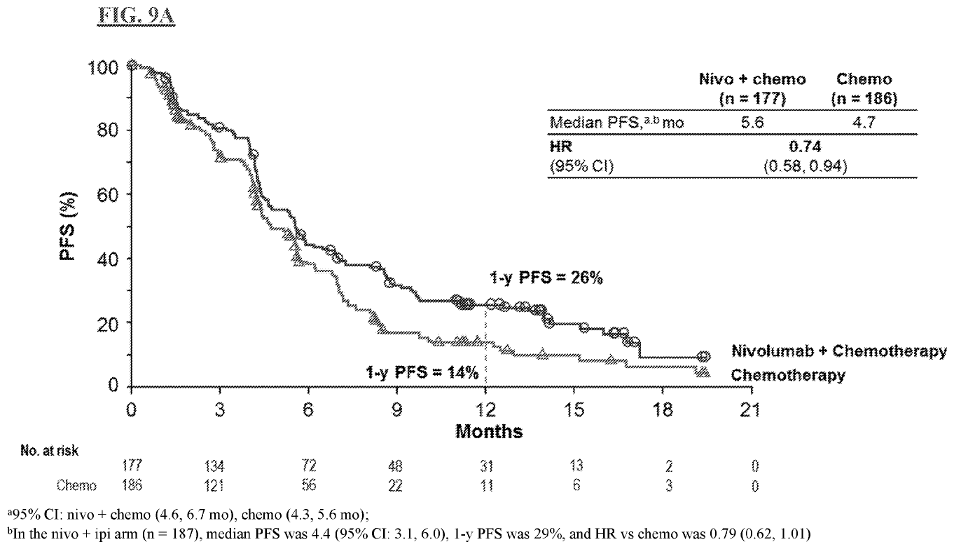

Among patients with TMB≥10 mutations/Mb and ≥1% PD-L1 expression, median PFS was 7.1 months (95% CI, 5.5 to 13.5) with nivolumab plus ipilimumab versus 4.2 months (95% CI, 2.6 to 8.3) with nivolumab monotherapy (HR, 0.75; 95% CI, 0.53 to 1.07). FIG. 8.

The results of this study demonstrate that in patients with advanced NSCLC and TMB≥10 mutations/Mb, first-line treatment with nivolumab plus ipilimumab is associated with improved PFS compared with chemotherapy. The benefit of combination immunotherapy was durable, with 43% of patients being progression free at 1 year (vs. 13% with chemotherapy) and 68% of responders having ongoing responses at 1 year (vs. 25% with chemotherapy). The benefit of nivolumab plus ipilimumab was observed in patients with ≥1% and <1% PD-L1 expression, squamous and nonsquamous histology, and was consistent across the majority of other subgroups. Although improved PFS was seen with nivolumab plus ipilimumab vs. chemotherapy in all randomized patients, TMB≥10 mutations/Mb was an effective biomarker. Benefit with nivolumab plus ipilimumab was particularly enhanced in those with high TMB while no benefit relative to chemotherapy was seen in those with low TMB (<10 mutations/Mb). Additionally, nivolumab plus ipilimumab had improved efficacy compared with nivolumab monotherapy in patients with TMB≥10 mutations/Mb, highlighting the distinct importance of dual immune-checkpoint blockade in NSCLC with TMB≥10 mutations/Mb. The study continues for the co-primary endpoint of OS in PD-L1-selected patients.

This study shows that the TMB and PD-L1 expressions were independent biomarkers. Among patients with high TMB, the benefit of nivolumab plus ipilimumab compared with chemotherapy was similar in patients with ≥1% and <1% tumor PD-L1 expression. Therefore, nivolumab plus ipilimumab represents a new, effective treatment regimen for patients with TMB≥10 mutations/Mb irrespective of PD-L1 expression.

Safety of nivolumab plus ipilimumab was consistent with previously reported data in first-line NSCLC. In a previous study, various dosing regimens of nivolumab plus ipilimumab were evaluated in 8 cohorts, and the nivolumab 3 mg/kg every 2 weeks plus ipilimumab 1 mg/kg every 6 weeks regimen was found to be well tolerated and effective. Hellmann, M. D., et al. Lancet Oncol, 18:31-41 (2017). These findings were confirmed in our large, international study, with no new safety signals observed with the combination. The rates of treatment-related select adverse events and treatment-related discontinuations were only modestly higher than those with nivolumab monotherapy, which was also well tolerated, with low rates of select adverse events.

Although the rates of treatment-related adverse events leading to discontinuation were higher with nivolumab plus ipilimumab than chemotherapy, this may in part be related to longer treatment durations and longer PFS with nivolumab plus ipilimumab.

Important questions remain regarding the role of immunotherapy/immunotherapy combinations versus immunotherapy/chemotherapy combinations, the optimal sequencing of therapies, whether TMB can identify patients who may derive benefit from immunotherapy/chemotherapy combinations, and whether an optimal TMB cutoff can be identified for PD-1/L1 monotherapy. Given that the results of our study validate the clinical utility of TMB as an important and independent biomarker, a concerted multidisciplinary effort will be necessary to ensure the availability of sufficient tumor tissue for testing and acceptable turnaround time. The 58% rate of TMB results reported in this study was mainly due to the limited availability of tumor samples of sufficient quantity or quality, a result of limited tissue requested for biomarker analysis as part of the study. In clinical practice, when the intent to test for TMB is known upfront and sufficient quantity and quality of tumor samples can be collected and submitted, successful TMB determination can be expected for 80% to 95% of patients undergoing testing.24 CheckMate 817 (NCT02869789), which will prospectively evaluate the feasibility of TMB testing for first-line nivolumab plus ipilimumab in patients with advanced NSCLC and TMB≥10 mutations/Mb, may help to identify gaps and opportunities in education to optimize the feasibility for TMB testing. Moreover, TMB is a reliable and reproducible biomarker that simultaneously provides comprehensive genomic profiling through next generation sequencing of multiple potentially therapeutically actionable cancer genes. Therefore, TMB testing leverages already routine technology to provide broadly applicable, clinically important information within a single test to guide management in first line NSCLC.

### Treatment Beyond Progression and Overall Survival Follow-Up

Treatment continuation with nivolumab or nivolumab plus ipilimumab beyond progression was permitted if the patient had investigator-assessed clinical benefit and continued to tolerate treatment. Patients were followed for overall survival every 3 months via in-person or phone contact after discontinuation of study drug treatment.

### Example 2: Nivolumab Plus Ipilimumab in Non-Small Cell Lung Cancer with <1% PD-L1 Expression

We report results from the phase 3 study of Example 1 for the co-primary endpoint of the efficacy and safety of nivolumab+ipilimumab and nivolumab+chemotherapy vs. chemotherapy in patients with <1% PD-L1 expression. Recent studies demonstrated the addition of anti-PD-(L)1 therapy to chemotherapy can improve outcomes as compared to chemotherapy alone. However, a lower magnitude of benefit was observed in patients with <1% PD-L1 expression (PFS HRs: 0.75 and 0.77) in non-squamous NSCLC.

Patients had chemotherapy-naive, stage IV or recurrent NSCLC. Those with ≥1% tumor PD-L1 expression were randomized 1:1:1 to nivolumab+ipilimumab, nivolumab, or chemotherapy; those with <1% tumor PD-L1 expression were randomized 1:1:1 to nivolumab+ipilimumab, nivolumab+chemotherapy, or chemotherapy (FIG. 1). TMB was determined using FOUNDATIONONE® CDX™. The secondary endpoints of the study included measuring progression free survival in patients with <1% tumor PD-L1 expression following treatment with nivolumab+chemotherapy as compared to chemotherapy alone, overall survival in PD-L1-selected populations with nivolumab+ipilimumab as compared to chemotherapy, and progression free survival in TMB-selected populations with nivolumab+ipilimumab as compared to chemotherapy.

A total of 550 patients in the study were identified as having <1% PD-L1 tumor expression, of which 177 were administered nivolumab+chemotherapy, 187 were administered nivolumab+ipilimumab, and 186 were administered chemotherapy. Table 24 shows the baseline characteristics of patients with <1% tumor PD-L1 expression.

TABLE 24
Baseline characteristics in Patients with <1% Tumor PD-L1
Expression

| | Nivolumab + | Nivolumab + | |
|---|---|---|---|
| | Chemotherapy | Ipilimumab | Chemotherapy |
| | (n = 177) | (n = 187) | (n = 186) |
| Median age, y | 64 | 63 | 64 |
| Female, % | 27 | 26 | 33 |
| ECOG PS, a % | | | |
| 0 | 33 | 37 | 31 |
| 1 | 66 | 63 | 68 |
| Smoking status, % | | | |
| Current/former | 84 | 87 | 85 |
| smoker | | | |
| Never smoker | 15 | 12 | 15 |
| Unknown | 1 | 1 | 0 |
| Histology, % | | | |
| Squamous | 24 | 25 | 25 |
| Non-squamous | 76 | 75 | 75 |
| TMB, % | | | |
| Evaluable | 55 | 48 | 58 |
| High (≥10 mut/Mb) | 44 | 42 | 45 |
| Low (<10 mut/Mb) | 56 | 58 | 55 |

### Results

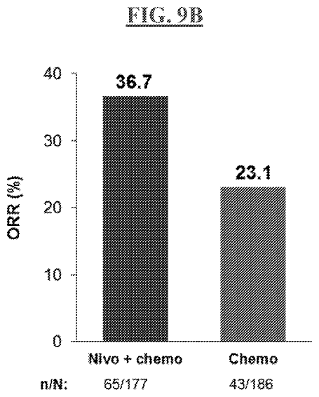

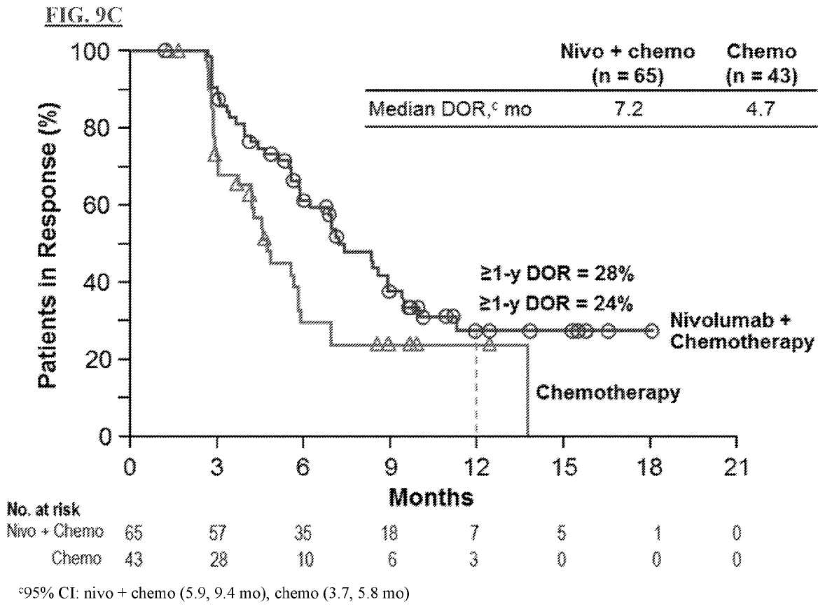

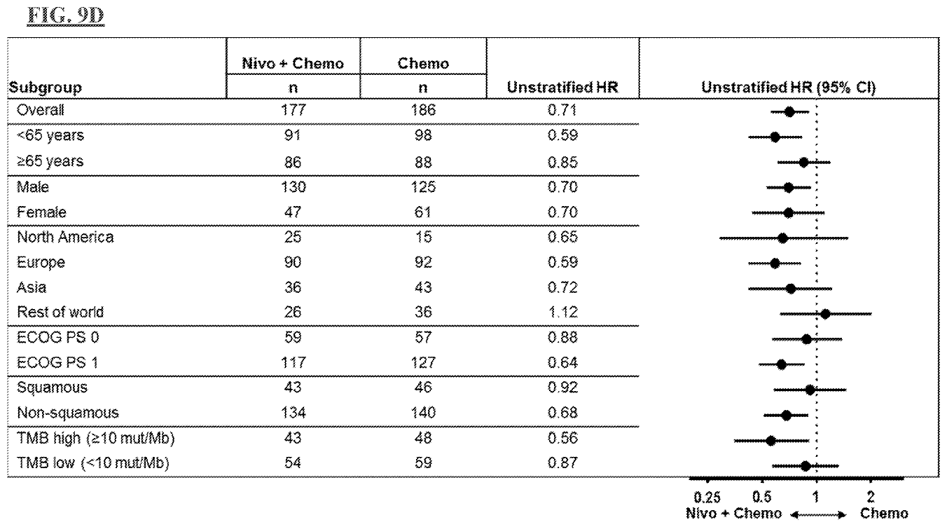

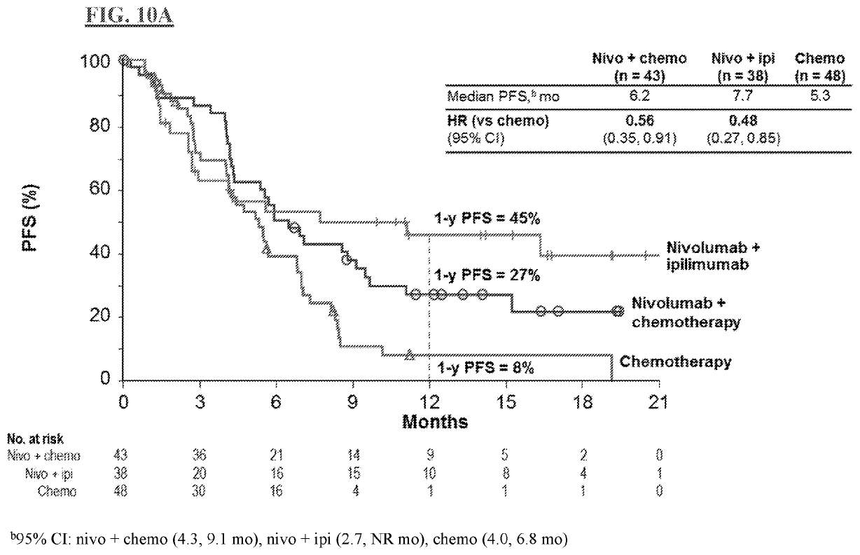

Patients with <10% tumor PD-L1 expression treated with nivolumab+chemotherapy had a progression free survival (PFS) rate of 26% at 1 year, whereas patients treated with chemotherapy alone had a 1-year PFS rate of 14% (FIG. 9A). The objected response rate for patients treated with nivolumab+chemotherapy was 36.7% as compared to 23.1% for patients treated with chemotherapy alone (FIG. 9B). The duration of response (DOR) for patients treated with nivolumab+chemotherapy was about 28% at 1 year as compared to about 24% for patients treated with chemotherapy alone (FIG. 9C). In addition, patients treated with nivolumab+ipilimumab had an ORR of about 25.1% and a median DOR of about 17.97 months (9500 CL: 12.2, NR) (data not shown).

Analysis of the patient population revealed that patients having a non-squamous NSCLC had a lower unstratified hazard ratio (HR; 0.68) than patients having squamous NSCLC (0.92) when comparing the responsiveness of the patients to treatment with nivolumab+chemotherapy and chemotherapy alone (FIG. 9D). Further, patients identified as being TMB high (≥10 mut/Mb) were found to have a lower unstratified HR (0.56) than TMB low (<10 mut/Mb) patients (0.87) (FIG. 9D).

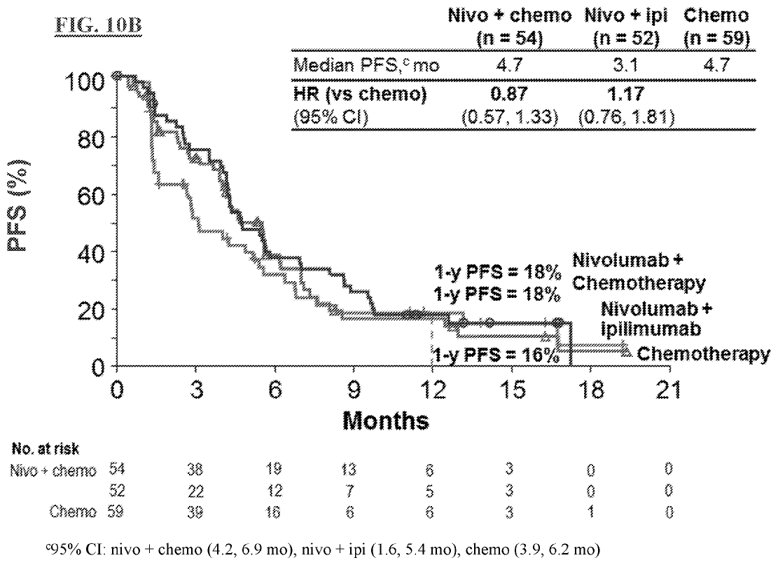

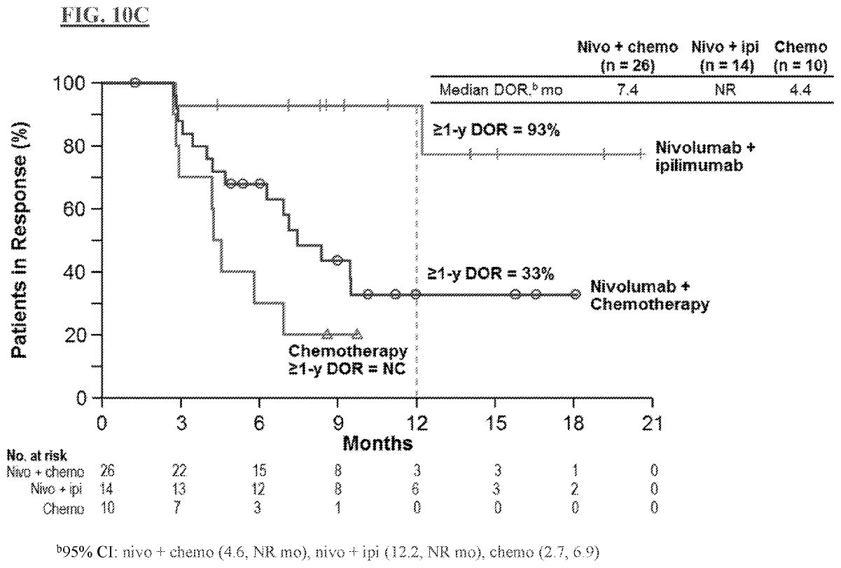

Patients were then stratified based on TMB status. High TMB (≥10 mut/Mb) patients having <1% tumor PD-L1 expression were found to have a 1-year PFS rates of about 45% following treatment with nivolumab+ipilimumab, about 27% following treatment with nivolumab+chemotherapy, and about 8% following treatment with chemotherapy alone (FIG. 10A). The median PFS was 7.7 months for patients treated with nivolumab+ipilimumab, 6.2 months for patients treated with nivolumab+chemotherapy, and 5.3 months in patients treated with chemotherapy alone (FIG. 10A).

Conversely, low TMB (<10 mut/Mb) patients having ≥1% tumor PD-L1 expression were found to have a 1-year PFS of about 18% following treatment with either nivolumab+ipilimumab or nivolumab+chemotherapy and a 1-year PFS of about 16% following treatment with chemotherapy alone (FIG. 10B). The median PFS was 3.1 months for patients treated with nivolumab+ipilimumab and 4.7 months for patients treated with either nivolumab+chemotherapy or chemotherapy alone (FIG. 10B).

The duration of response (DOR) for each treatment group was also measured. High TMB patients having <1% tumor PD-L1 expression showed 1-year DOR rates of about 93% following treatment with nivolumab+ipilimumab and about 33% following treatment with nivolumab+chemotherapy (FIG. 10C). The 1-year mark was not reached in the group of patients treated with chemotherapy alone (FIG. 10C). The median DOR for was 7.4 months for patients treated with nivolumab+chemotherapy and 4.4 months in patients treated with chemotherapy alone (FIG. 10C). The median DOR was not reached for patients treated with nivolumab+ipilimumab (FIG. 10C). The objective response rate for these treatment groups was 60.5% following treatment with nivolumab+chemotherapy, about 36.8% following treatment with nivolumab+ipilimumab, and about 20.8% following treatment with chemotherapy alone (data not shown). This difference was considerably greater than in low TMB patients having <1% tumor PD-L1 expression, who showed an ORR of 27.8% following treatment with nivolumab+chemotherapy and 22.0% following treatment with chemotherapy alone (data not shown).

### Safety

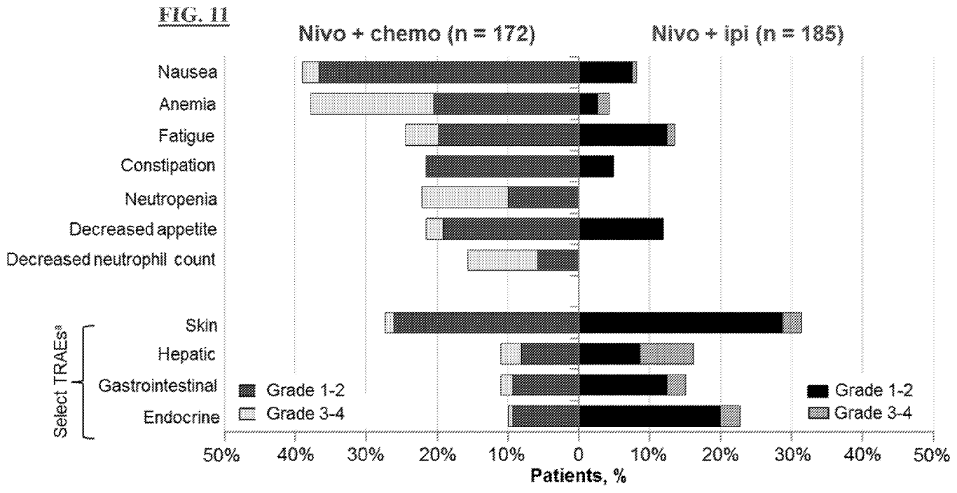

Treatment-related adverse events (TRAEs) are summarized in Table 25 and FIG. 11. There were four treatment-related deaths in the nivolumab+chemotherapy arm, three treatment-related deaths in the nivolumab+ipilimumab arm, and six treatment related deaths in the chemotherapy arms. Treatment-related adverse events in the chemotherapy arm were similar to nivolumab+chemotherapy arm and consistent with prior reports (FIG. 11).

TABLE 25
Treatment-Related Adverse Events

| | Nivolumab + | | Nivolumab + | | | |
|---|---|---|---|---|---|---|
| | Chemotherapy | | Ipilimumab | | Chemotherapy | |
| | (n = 172) | | (n = 185) | | (n = 183) | |
| | Any grade | Grade 3-4 | Any grade | Grade 3-4 | Any grade | Grade 3-4 |
| Any TRAE,^{a} % | 92 | 52 | 74 | 25 | 77 | 35 |
| TRAE leading to | 13 | 8 | 16 | 10 | 14 | 9 |
| discontinuation,^{b} % | | | | | | |

| | | | |
|---|---|---|---|
| Median number of doses | 8.5 for nivo (Q3W) | 8.0 for nivo (Q2W) | 4-7^{c} for chemo (Q3W) |
| received, n | 4-7^{c} for chemo (Q3W) | 3.0 for ipi (Q6W) | |

^{a}Includes events reported between first dose and 30 days after last dose of study drug;
^{b}For nivo + ipi, these events include TRAEs leading to discontinuation of ipi or both study drugs (patients could not discontinue nivo without discontinuing ipi); for nivo + chemo, patients who discontinued nivo or chemo or both were counted as having a TRAE leading to discontinuation;
^{c}In each treatment arm: gemcitabine 7, cisplatin 4, carboplatin 4, and pemetrexed 7 (nivo + chemo) and 6 (chemo);
^{d}Chemo arms, n = 570 (Part 1a, n = 387; Part 1b, n = 183)

Nivolumab+chemotherapy vs chemotherapy alone PFS HR was observed to be 0.74 (95% CI: 0.58, 0.94; NSQ PFS HR=0.68, 95% CI: 0.51, 0.90) in patients with <1% PD-L1 expression, consistent with other PD-(L)1+chemotherapy studies. TMB testing is clinically relevant for selecting patients for immunoncology+immunoncology and immunoncology+chemotherapy. PFS benefit from nivolumab+chemotherapy vs chemotherapy alone is enhanced in patients with high TMB (≥10 mut/Mb) and <1% PD-L1 expression. Patients with low TMB (<10 mut/Mb) and <1% PD-L1 do not derive PFS benefit from immunoncology+immunoncology and immunoncology+chemotherapy. In addition, there are fewer grade 3/4 TRAEs with a potentially favorable safety profile for immunoncology+immunoncology and immunoncology+chemotherapy.

All publications, patents, and patent applications disclosed herein are incorporated by reference to the same extent as if each individual publication, patent or patent application was specifically and individually indicated to be incorporated by reference.

This application claims the benefit of U.S. Provisional Application Nos. 62/650,845, filed Mar. 30, 2018, and 62/671,906, filed May 15, 2018, which are incorporated by reference herein in their entireties.

1. A method of treating a subject afflicted with a non-small cell lung cancer (NSCLC) comprising administering to the subject:
(i) a flat dose of 240 mg or 480 mg of an antibody or antigen-binding portion thereof that specifically binds to a Programmed Death-1 (PD-1) receptor and inhibits PD-1 activity (“an anti-PD-1 antibody”); and
(ii) an antibody or antigen-binding portion thereof that binds specifically to cytotoxic T-lymphocyte-associated protein 4 (CTLA-4) (“an anti-CTLA-4 antibody”),
wherein the tumor has less than 1% expression of the PD-L1; and wherein the tumor has a tumor mutation burden (TMB) status of at least about 10 mutations per megabase of genes examined, wherein the TMB status is determined by genomic profiling, and wherein the genomic profile comprises the genes ABL1, BRAF, CHEK1, FANCC, GATA3, JAK2, MITF, PDCD1LG2 (PD-L2), RBM10, STAT4, ABL2, BRCA1, CHEK2, FANCD2, GATA4, JAK3, MLH1, PDGFRA, RET, STK11, ACVR1B, BRCA2, CIC, FANCE, GATA6, JUN, MPL, PDGFRB, RICTOR, SUFU, AKT1, BRD4, CREBBP, FANCE, GID4 (C17orf39), KAT6A (MYST 3), MRE 11A, PDK1, RNF43, SYK, AKT2, BRIP1, CRKL, FANCG, GLl1, KDM5A, MSH2, PIK3C2B, ROS1, TAF1, AKT3, BTG1, CRLF2, FANCL, GNA11, KDM5C, MSH6, PIK3CA, RPTOR, TBX3, ALK, BTK, CSF1R, FAS, GNA13, KDM6A, MTOR, PIK3CB, RUNX1, TERC, AMER1 (FAM123B), C11orf 30 (EMSY), CTCF, FAT1, GNAQ, KDR, MUTYH, PIK3CG, RUNX1T1, TERT (Promoter only), APC, CARD11, CTNNA1, FBXW7, GNAS, KEAP1, MYC, PIK3R1, SDHA, TET2, AR, CBFB, CTNN B1, FGF10, GPR124, KEL, MYCL (MYC L1), PIK3R2, SDHB, TGFBR2, ARAF, CBL, CUL3, FGF14, GRIN2A, KIT, MYCN, PLCG2, SDHC, TNFAIP3, ARFRP1, CCND1, CYLD, FGF19, GRM3, KLHL6, MYD88, PMS2, SDHD, TNFRSF14, ARID1A, CCND2, DAXX FGF23, GSK3B, KMT2A (MLL), NF1, POLD1, SETD2, TOP1, ARID1B, CCND3, DDR2, FGF3, H3F3A, KMT2C (MLL3), NF2, POLE, SF3B1, TOP2A, ARID2, CCNE1, DICER1, FGF4, HGF, KMT2D (MLL2), NFE2L2, PPP2R1A, SLIT2, TP53, ASXL1, CD274 (PD-L1), DNMT3A, FGF6, HNF1A, KRAS, NFKB1A, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX, CDC73, EP300, FGFR3, HSP90AA1, LYN, NOTCH2, PRKC1, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLCN, IGF1R, MAP2K1 (MEK1), NRAS, PTCH1, SNCAIP, WISP3, AXL, CDK6, EPHB1, FLT1, IGF2, MAP2K2 (MEK2), NSD1, PTEN, SOCS1, WT1, BAP1, CDK8, ERBB2, FLT3, IKBKE, MAP2K4, NTRK1, PTPN11, SOX10, XPO1, BARD1, CDKN1A, ERBB3, FLT4, IKZF1, MAP3K1, NTRK2, OKI, SOX2, ZBTB2, BCL2, CDKN1B, ERBB4, FOXL2, IL7R, MCL1, NTRK3, RAC1, SOX9, ZNF217, BCL2L1, CDKN2A, ERG, FOXP1, INHBA, MDM2, NUP93, RAD50, SPEN, ZNF703, BCL2L2, CDKN2B, ERRFl1, FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRM1, RB1, and STAT3.

2. The method of claim 1, wherein the TMB status is measured in a biological sample obtained from the subject prior to the administering.

3. The method of claim 1, wherein the TMB status is determined by sequencing nucleic acids in the tumor and identifying a genomic alteration in the sequenced nucleic acids.

4. The method of claim 3, wherein the genomic alteration comprises
one or more somatic mutations selected from
(i) one or more nonsynonymous mutations;
(ii) one or more missense mutations;
(iii) one or more alterations selected from the group consisting of a base pair substitution, a base pair insertion, a base pair deletion, a copy number alteration (CNAs), a gene rearrangement, and any combination thereof; or
(v) any combination of (i)-(iii).

5. The method of claim 1, wherein genomic profile consists of the genes ABL1, BRAF, CHEK1, FANCC, GATA3, JAK2, MITF, PDCD1LG2 (PD-L2), RBM10, STAT4, ABL2, BRCA1, CHEK2, FANCD2, GATA4, JAK3, MLH1, PDGFRA, RET, STK11, ACVR1B, BRCA2, CIC, FANCE, GATA6, JUN, MPL, PDGFRB, RICTOR, SUFU, AKT1, BRD4, CREBBP, FANCF, GID4 (C17orf39), KAT6A (MYST 3), MRE HA, PDK1, RNF43, SYK, AKT2, BRIP1, CRKL, FANCG, GLl1, KDM5A, MSH2, PIK3C2B, ROS1, TAF1, AKT3, BTG1, CRLF2, FANCL, GNA11, KDM5C, MSH6, PIK3CA, RPTOR, TBX3, ALK, BTK, CSF1R, FAS, GNA13, KDM6A, MTOR, PIK3CB, RUNX1, TERC, AMER1 (FAM123B), C11orf 30 (EMSY), CTCF, FAT1, GNAQ, KDR, MUTYH, PIK3CG, RUNX1T1, TERT (Promoter only), APC, CARD11, CTNNA1, FBXW7, GNAS, KEAP1, MYC, PIK3R1, SDHA, TET2, AR, CBFB, CTNN B1, FGF10, GPR124, KEL, MYCL (MYC L1), PIK3R2, SDHB, TGFBR2, ARAF, CBL, CUL3, FGF14, GRIN2A, KIT, MYCN, PLCG2, SDHC, TNFAIP3, ARFRP1, CCND1, CYLD, FGF19, GRM3, KLHL6, MYD88, PMS2, SDHD, TNFRSF14, ARID1A, CCND2, DAXX FGF23, GSK3B, KMT2A (MLL), NF1, POLD1, SETD2, TOP1, ARID1B, CCND3, DDR2, FGF3, H3F3A, KMT2C (MLL3), NF2, POLE, SF3B1, TOP2A, ARID2, CCNE1, DICER1, FGF4, HGF, KMT2D (MLL2), NFE2L2, PPP2R1A, SLIT2, TP53, ASXL1, CD274 (PD-L1), DNMT3A, FGF6, HNF1A, KRAS, NFKB1A, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX, CDC73, EP300, FGFR3, HSP90AA1, LYN, NOTCH2, PRKC1, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLCN, IGF1R, MAP2K1 (MEK1), NRAS, PTCH1, SNCAIP, WISP3, AXL, CDK6, EPHB1, FLT1, IGF2, MAP2K2 (MEK2), NSD1, PTEN, SOCS1, WT1, BAP1, CDK8, ERBB2, FLT3, IKBKE, MAP2K4, NTRK1, PTPN11, SOX10, XPO1, BARD1, CDKN1A, ERBB3, FLT4, IKZF1, MAP3K1, NTRK2, OKI, SOX2, ZBTB2, BCL2, CDKN1B, ERBB4, FOXL2, IL7R, MCL1, NTRK3, RAC1, SOX9, ZNF217, BCL2L1, CDKN2A, ERG, FOXP1, INHBA, MDM2, NUP93, RAD50, SPEN, ZNF703, BCL2L2, CDKN2B, ERRFl1, FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRM1, RB1, and STAT3.

6. The method of claim 2, wherein the biological sample comprises a tumor tissue biopsy, a liquid biopsy, blood, serum, plasma, exoRNA, circulating tumor cells, ctDNA, cfDNA, or any combination thereof.

7. The method of claim 1, wherein the TMB status is determined by:
(i) genome sequencing,
(ii) exome sequencing,
(iii) genomic profiling, or
(iv) any combination of (i)-(iii).

8-10. (canceled)

11. The method of claim 1, wherein the anti-PD-1 antibody is administered at a weight-based dose ranging from 0.1 mg/kg to 20.0 mg/kg body weight or at a flat dose of at least about 200 mg once every 2, 3, or 4 weeks.

12. A method of treating a subject afflicted with a non-small cell lung cancer (NSCLC) comprising administering to the subject:
(i) a dose of an antibody or antigen-binding portion thereof that specifically binds to a Programmed Death-1 (PD-1) receptor and inhibits PD-1 activity (“an anti-PD-1 antibody”) or an antibody or antigen-binding portion thereof that binds specifically to a Programmed Death-Ligand 1 (PD-L1) and inhibits PD-1 activity (“an anti-PD-L1 antibody”); and for use in the treatment of a subject afflicted with a tumor derived from a non-small cell lung cancer (NSCLC) in combination with
(ii) an antibody or antigen-binding portion thereof that binds specifically to cytotoxic T-lymphocyte-associated protein 4 (CTLA-4) (“an anti-CTLA-4 antibody”),
wherein the tumor has less than 1% expression of the PD-L1; and wherein the tumor has a tumor mutation burden (TMB) status of at least about 10 mutations per megabase of genes examined, wherein the TMB status is determined by genomic profiling, and wherein the genomic profile comprises the genes ABL1, BRAF, CHEK1, FANCC, GATA3, JAK2, MITF, PDCD1LG2 (PD-L2), RBM10, STAT4, ABL2, BRCA1, CHEK2, FANCD2, GATA4, JAK3, MLH1, PDGFRA, RET, STK11, ACVR1B, BRCA2, CIC, FANCE, GATA6, JUN, MPL, PDGFRB, RICTOR, SUFU, AKT1, BRD4, CREBBP, FANCF, GID4 (C17orf 39), KAT6A (MYST 3), MRE 11A, PDK1, RNF43, SYK, AKT2, BRIP1, CRKL, FANCG, GLl1, KDM5A, MSH2, PIK3C2B, ROS1, TAF1, AKT3, BTG1, CRLF2, FANCL, GNA11, KDM5C, MSH6, PIK3CA, RPTOR, TBX3, ALK, BTK, CSF1R, FAS, GNA13, KDM6A, MTOR, PIK3CB, RUNX1, TERC, AMER1 (FAM123B), C11orf 30 (EMSY), CTCF, FAT1, GNAQ, KDR, MUTYH, PIK3CG, RUNX1T1, TERT (Promoter only), APC, CARD11, CTNNA1, FBXW7, GNAS, KEAP1, MYC, PIK3R1, SDHA, TET2, AR, CBFB, CTNN B1, FGF10, GPR124, KEL, MYCL (MYC L1), PIK3R2, SDHB, TGFBR2, ARAF, CBL, CUL3, FGF14, GRIN2A, KIT, MYCN, PLCG2, SDHC, TNFAIP3, ARFRP1, CCND1, CYLD, FGF19, GRM3, KLHL6, MYD88, PMS2, SDHD, TNFRSF14, ARID1A, CCND2, DAXX, FGF23, GSK3B, KMT2A (MLL), NF1, POLD1, SETD2, TOP1, ARID1B, CCND3, DDR2, FGF3, H3F3A, KMT2C (MLL3), NF2, POLE, SF3B1, TOP2A, ARID2, CCNE1, DICER1, FGF4, HGF, KMT2D (MLL2), NFE2L2, PPP2R1A, SLIT2, TP53, ASXL1, CD274 (PD-L1), DNMT3A, FGF6, HNF1A, KRAS, NFKB1A, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX, CDC73, EP300, FGFR3, HSP90AA1, LYN, NOTCH2, PRKC1, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLCN, IGF1R, MAP2K1 (MEK1), NRAS, PTCH1, SNCAIP, WISP3, AXL, CDK6, EPHB1, FLT1, IGF2, MAP2K2 (MEK2), NSD1, PTEN, SOCS1, WT1, BAP1, CDK8, ERBB2, FLT3, IKBKE, MAP2K4, NTRK1, PTPN11, SOX10, XPO1, BARD1, CDKN1A, ERBB3, FLT4, IKZF1, MAP3K1, NTRK2, QKI, SOX2, ZBTB2, BCL2, CDKN1B, ERBB4, FOXL2, IL7R, MCL1, NTRK3, RAC1, SOX9, ZNF217, BCL2L1, CDKN2A, ERG, FOXP1, INHBA, MDM2, NUP93, RAD50, SPEN, ZNF703, BCL2L2, CDKN2B, ERRFl1, FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRM1, RB1, and STAT3,
wherein the anti-PD-1 antibody is administered:
(i) at a dose of 2 mg/kg body weight once every 3 weeks;
(ii) at a dose of 3 mg/kg body weight once every 2 weeks; or
(iii) at a flat dose of about 200 mg once every 2 weeks; or

13. The method of claim 1, wherein the anti-CTLA-4 antibody is administered at a weight-based dose ranging from 0.1 mg/kg to 20.0 mg/kg body weight or at a flat dose of at least about 40 mg once every 2, 3, 4, 5, 6, 7, or 8 weeks.

14. The method of claim 1, wherein the anti-CTLA-4 antibody is administered:
(i) at a dose of 1 mg/kg body weight once every 6 weeks;
(ii) at a dose of 1 mg/kg body weight once every 4 weeks; or
(iii) at a flat dose of at least about 80 mg once every 4 weeks.

15. (canceled)

16. The method of claim 1, wherein the anti-PD-1 antibody comprises nivolumab.

17. The method of claim 1, wherein the anti-CTLA-4 antibody comprises ipilimumab or tremelimumab.

18. The method of claim 16, wherein the anti-CTLA-4 antibody comprises ipilimumab.

19. A method of treating a subject afflicted with a non-small cell lung cancer (NSCLC) comprising administering to the subject:
(i) an antibody or antigen-binding portion thereof that specifically binds to a Programmed Death-1 (PD-1) receptor and inhibits PD-1 activity (“an anti-PD-1 antibody”) or an antibody or antigen-binding portion thereof that binds specifically to a Programmed Death-Ligand 1 (PD-L1) and inhibits PD-1 activity (“an anti-PD-L1 antibody”); and
(ii) an antibody or antigen-binding portion thereof that binds specifically to cytotoxic T-lymphocyte-associated protein 4 (CTLA-4) (“an anti-CTLA-4 antibody”),
wherein the tumor has less than 1% expression of the PD-L1; and wherein the tumor has a tumor mutation burden (TMB) status of at least about 10 mutations per megabase of genes examined, wherein the TMB status is determined by genomic profiling, and wherein the genomic profile comprises the genes ABL1, BRAF, CHEK1, FANCC, GATA3, JAK2, MITF, PDCD1LG2 (PD-L2), RBM10, STAT4, ABL2, BRCA1, CHEK2, FANCD2, GATA4, JAK3, MLH1, PDGFRA, RET, STK11, ACVR1B, BRCA2, CIC, FANCE, GATA6, JUN, MPL, PDGFRB, RICTOR, SUFU, AKT1, BRD4, CREBBP, FANCF, GID4 (C17orf 39), KAT6A (MYST 3), MRE 11A, PDK1, RNF43, SYK, AKT2, BRIP1, CRKL, FANCG, GLl1, KDM5A, MSH2, PIK3C2B, ROS1, TAF1, AKT3, BTG1, CRLF2, FANCL, GNA11, KDM5C, MSH6, PIK3CA, RPTOR, TBX3, ALK, BTK, CSF1R, FAS, GNA13, KDM6A, MTOR, PIK3CB, RUNX1, TERC, AMER1 (FAM123B), C11orf 30 (EMSY), CTCF, FAT1, GNAQ, KDR, MUTYH, PIK3CG, RUNX1T1, TERT (Promoter only), APC, CARD11, CTNNA1, FBXW7, GNAS, KEAP1, MYC, PIK3R1, SDHA, TET2, AR, CBFB, CTNN B1, FGF10, GPR124, KEL, MYCL (MYC L1), PIK3R2, SDHB, TGFBR2, ARAF, CBL, CUL3, FGF14, GRIN2A, KIT, MYCN, PLCG2, SDHC, TNFAIP3, ARFRP1, CCND1, CYLD, FGF19, GRM3, KLHL6, MYD88, PMS2, SDHD, TNFRSF14, ARID1A, CCND2, DAXX FGF23, GSK3B, KMT2A (MLL), NF1, POLD1, SETD2, TOP1, ARID1B, CCND3, DDR2, FGF3, H3F3A, KMT2C (MLL3), NF2, POLE, SF3B1, TOP2A, ARID2, CCNE1, DICER1, FGF4, HGF, KMT2D (MLL2), NFE2L2, PPP2R1A, SLIT2, TP53, ASXL1, CD274 (PD-L1), DNMT3A, FGF6, HNF1A, KRAS, NFKB1A, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX CDC73, EP300, FGFR3, HSP90AA1, LYN, NOTCH2, PRKC1, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLCN, IGF1R, MAP2K1 (MEK1), NRAS, PTCH1, SNCAIP, WISP3, AXL, CDK6, EPHB1, FLT1, IGF2, MAP2K2 (MEK2), NSD1, PTEN, SOCS1, WT1, BAP1, CDK8, ERBB2, FLT3, IKBKE, MAP2K4, NTRK1, PTPN11, SOX10, XPO1, BARD1, CDKN1A, ERBB3, FLT4, IKZF, MAP3K1, NTRK2, QKI, SOX2, ZBTB2, BCL2, CDKN1B, ERBB4, FOXL2, IL7R, MCL1, NTRK3, RAC1, SOX9, ZNF217, BCL2L1, CDKN2A, ERG, FOXP1, INHBA, MDM2, NUP93, RAD50, SPEN, ZNF703, BCL2L2, CDKN2B, ERRFl1, FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRM1, RB1, and STAT3;
wherein the anti-PD-1 antibody is administered at a dose of 3 mg/kg body weight once every 2 weeks, and wherein the anti-CTLA-4 antibody is administered at a dose of 1 mg/kg body weight once every 6 weeks.

20. The method of claim 19, wherein the anti-PD-1 antibody comprises nivolumab.

21. The method of claim 19, wherein the anti-CTLA-4 antibody comprises ipilimumab or tremelimumab.

22. The method of claim 20, wherein the anti-CTLA-4 antibody comprises ipilimumab.

23. A kit for treating a patient afflicted with a tumor derived from a non-small cell lung cancer (NSCLC), the kit comprising:
(a) a dosage ranging from 0.1 mg/kg to 10.0 mg/kg body weight of the anti-PD-1 antibody or a dosage ranging from 0.1 to 20 mg/kg body weight of an anti-PD-L1 antibody;
(b) a dosage ranging from 0.1 mg/kg to 10.0 mg/kg body weight of the anti-CTLA-4 antibody; and
(c) instructions for using (a) the anti-PD-1 antibody or the anti-PD.L1 antibody and (b) the anti-CTLA-4 antibody in the method of claim 1.


SEQUENCE LISTING (10 sequences)

SEQ ID NO: 1 - protein, 123 aa, synthetic construct
  source 1..123: note = anti-GITR VH
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVS

SEQ ID NO: 2 - protein, 107 aa, synthetic construct
  source 1..107: note = anti-GITR VL
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 3 - protein, 123 aa, synthetic construct
  source 1..123: note = anti-GITR VH
QVQLVESGGGVVQPGRSLRLSCAASGFTFSSYGFHWVRQAPGKGLEWVAVIWYAGSNKFYADSVKGRFTISRDNSKNTLYLQMNSLRAEDTAVYYCARGGQLDYYYYYVMDVWGQGTTVTVSS

SEQ ID NO: 4 - protein, 107 aa, synthetic construct
  source 1..107: note = anti-GITR VL
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 5 - protein, 122 aa, synthetic construct
  source 1..122: note = anti-GITR VH
VQLVESGGGVVQPGRSLRLSCAASGFTFSSYGMHWVRQAPGKGLEWVAVIWYAGSNKYYADSVKGRFTISRDNSKNTLYLQMNSLRAEDTAVYYCARGGRIAVAFYYSMDVWGQGTTVTVSS

SEQ ID NO: 6 - protein, 107 aa, synthetic construct; identical to SEQ ID NO: 4
  source 1..107: note = anti-GITR VL

SEQ ID NO: 7 - protein, 449 aa, synthetic construct
  source 1..449: note = anti-GITR heavy chain
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 8 - protein, 214 aa, synthetic construct
  source 1..214: note = Anti-GITR light chain
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 453 aa, synthetic construct
  source 1..453: note = anti-GITR heavy chain
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct; identical to SEQ ID NO: 8
  source 1..214: note = anti-GITR light chain